SEQUENCE LISTING (26 sequences)

SEQ ID NO: 1 - protein, 286 aa, Escherichia coli
MEDLNVVDSINGAGSWLVANQALLLSYAVNIVAALAIIIVGLIIARMISNAVNRLMISRKIDATVADFLSALVRYGIIAFTLIAALGRVGVQTASVIAVLGAAGLAVGLALQGSLSNLAAGVLLVMFRPFRAGEYVDLGGVAGTVLSVQIFSTTMRTADGKIIVIPNGKIIAGNIINFSREPVRRNEFIIGVAYDSDIDQVKQILTNIIQSEDRILKDREMTVRLNELGASSINFVVRVWSNSGDLQNVYWDVLERIKREFDAAGISFPYPQMDVNFKRVKEDKAA

SEQ ID NO: 2 - DNA, 858 bp, Escherichia coli
  source 1..858: mol_type = genomic DNA
atggaagatttgaatgttgtcgatagcataaacggcgcgggaagctggctggtagctaaccaggcgctgctgctaagttatgcagtaaacatcgtggcggcactcgcgatcatcatcgttggtttgattatcgcgcggatgatttccaacgcggtgaatcgcctgatgatctcccgtaaaatcgatgccactgttgctgattttctttctgcattagtccgttacggtattatcgcctttacgctaatcgcagccctgggacgcgtgggtgtacaaacagcgtcagtcattgctgtactcggtgccgcaggcttagctgttggtctggctttgcaggggtcactttctaacctggccgctggcgtgttacttgtcatgttccgcccgttccgtgccggagaatatgttgacctcggcggcgtagccggtactgtgctgagtgtgcagattttctccaccaccatgcgtactgcagacggtaaaattatcgttattccgaacggtaaaattattgccggaaatattattaacttctcccgcgagccagttcgccgtaacgaatttattattggcgtggcgtatgattccgatatcgatcaggttaagcagatcctgaccaatattatccagtctgaagatcgcattttgaaagatcgcgaaatgactgtgcgcctgaacgaacttggtgcatcgtcgattaatttcgtggtccgcgtctggagcaacagcggcgatctgcaaaacgtgtactgggatgtgctggagcgtattaaacgtgaatttgatgccgccggtatcagcttcccgtacccgcaaatggatgtgaactttaagcgggtgaaagaagacaaagctgcg

SEQ ID NO: 3 - DNA, 861 bp, synthetic construct
  misc_feature 1..861: note = Description of artificial sequence: synthetic polynucleotide sequence
  source 1..861: mol_type = other DNA
atggaggatctgaatgtcgtggactctatcaatggggccgggtcttggctggtcgctaaccaggcactgctgctgagctatgctgtgaacattgtggccgctctggccatcattatcgtcgggctgattatcgctcgcatgatcagtaacgcagtgaatagactgatgatttcaaggaagatcgatgctaccgtggcagacttcctgtctgccctggtcagatacggcattatcgcttttactctgattgcagccctgggaagggtgggcgtccagaccgcctccgtgatcgctgtcctgggagctgcaggactggcagtgggcctggccctgcaggggtctctgagtaatctggccgctggcgtgctgctggtcatgttcaggccctttcgcgcaggggagtatgtggatctgggcggggtcgctggaacagtgctgtccgtccagattttctctaccacaatgcgcactgccgacggaaagattatcgtgatccccaacggaaaaattatcgccggcaacattatcaatttcagccgagagcctgtgcggagaaatgagttcatcatcggcgtggcctacgacagcgatattgaccaggtgaagcagatcctgaccaacattatccagtccgaagatcgcatcctgaaagaccgagagatgacagtgcggctgaacgaactgggcgccagctccatcaatttcgtggtcagagtgtggtcaaacagcggggatctgcagaacgtgtactgggacgtgctggagcggatcaagcgggaatttgatgcagccggcatctcattcccctacccacagatggatgtcaacttcaagagagtcaaggaggataaagccgcttaa

SEQ ID NO: 4 - protein, 136 aa, Escherichia coli
MSIIKEFREFAMRGNVVDLAVGVIIGAAFGKIVSSLVADIIMPPLGLLIGGIDFKQFAVTLRDAQGDIPAVVMHYGVFIQNVFDFLIVAFAIFMAIKLINKLNRKKEEPAAAPAPTKEEVLLTEIRDLLKEQNNRS

SEQ ID NO: 5 - DNA, 411 bp, Escherichia coli
  source 1..411: mol_type = genomic DNA
atgagcattattaaagaatttcgcgaatttgcgatgcgcgggaacgtggtggatttggcggtgggtgtcattatcggtgcggcattcgggaagattgtatcttcactggttgccgatatcatcatgccaccactgggcttattaattggcgggatcgactttaaacagtttgctgtcacgctacgcgatgcgcagggggatatccctgctgttgtgatgcattacggtgtcttcattcaaaacgtctttgattttctgattgtggcctttgccatctttatggcgattaagctaatcaacaaactgaatcggaaaaaagaagaaccagcagccgcaccagcaccaactaaagaagaagtattactgacagaaattcgtgatttgctgaaagagcagaataaccgctcttaa

SEQ ID NO: 6 - DNA, 411 bp, synthetic construct
  misc_feature 1..411: note = Description of artificial sequence: synthetic polynucleotide sequence
  source 1..411: mol_type = other DNA
atgtctatcatcaaggagttcagggagtttgccatgagaggaaacgtggtggacctggcagtgggcgtgatcatcggagcagccttcggcaagatcgtgagctccctggtggccgacatcatcatgccacctctgggactgctgatcggaggcatcgatttcaagcagtttgcagtgaccctgagggacgcacagggcgatatccctgcagtggtcatgcactacggcgtgtttatccagaacgtgttcgactttctgatcgtggccttcgccatctttatggccatcaagctgatcaacaagctgaatcggaagaaggaggagccagcagcagcaccagcaccaaccaaggaggaggtgctgctgacagagatccgggatctgctgaaggagcagaacaatcgcagctga

SEQ ID NO: 7 - protein, 1120 aa, Escherichia coli
MTMFQYYKRSRHFVFSAFIAFVFVLLCQNTAFARASSNGDLPTKADLQAQLDSLNKQKDLSAQDKLVQQDLTDTLATLDKIDRIKEETVQLRQKVAEAPEKMRQATAALTALSDVDNDEETRKILSTLSLRQLETRVAQALDDLQNAQNDLASYNSQLVSLQTQPERVQNAMYNASQQLQQIRSRLDGTDVGETALRPSQKVLMQAQQALLNAEIDQQRKSLEGNTVLQDTLQKQRDYVTANSARLEHQLQLLQEAVNSKRLTLTEKTAQEAVSPDEAARIQANPLVKQELEINQQLSQRLITATENGNQLMQQNIKVKNWLERALQSERNIKEQIAVLKGSLLLSRILYQQQQTLPSADELENMTNRIADLRLEQFEVNQQRDALFQSDAFVNKLEEGHTNEVNSEVHDALLQVVDMRRELLDQLNKQLGNQLMMAINLQINQQQLMSVSKNLKSILTQQIFWVNSNRPMDWDWIKAFPQSLKDEFKSMKITVNWQKAWPAVFIAFLAGLPLLLIAGLIHWRLGWLKAYQQKLASAVGSLRNDSQLNTPKAILIDLIRALPVCLIILAVGLILLTMQLNISELLWSFSKKLAIFWLVFGLCWKVLEKNGVAVRHFGMPEQQTSHWRRQIVRISLALLPIHFWSVVAELSPLHLMDDVLGQAMIFFNLLLIAFLVWPMCRESWRDKESHTMRLVTITVLSIIPIALMVLTATGYFYTTLRLAGRWIETVYLVIIWNLLYQTVLRGLSVAARRIAWRRALARRQNLVKEGAEGAEPPEEPTIALEQVNQQTLRITMLLMFALFGVMFWAIWSDLITVFSYLDSITLWHYNGTEAGAAVVKNVTMGSLLFAIIASMVAWALIRNLPGLLEVLVLSRLNMRQGASYAITTILNYIIIAVGAMTVFGSLGVSWDKLQWLAAALSVGLGFGLQEIFGNFVSGLIILFERPVRIGDTVTIGSFSGTVSKIRIRATTITDFDRKEVIIPNKAFVTERLINWSLTDTTTRLVIRLGVAYGSDLEKVRKVLLKAATEHPRVMHEPMPEVFFTAFGASTLDHELRLYVRELRDRSRTVDELNRTIDQLCRENDINIAFNQLEVHLHNEKGDEVTEVKRDYKGDDPTPAVG

SEQ ID NO: 8 - DNA, 3360 bp, Escherichia coli
  source 1..3360: mol_type = genomic DNA
ccctaccgctggcgtcgggtcatcgcctttgtagtcgcgttttacttccgtcacctcatcgcccttctcgttgtgcagatgcacttcaagctggttaaaggcaatgttgatgtcgttttcacggcacagctgatcgatagtacggttcagctcatcgacagtacgactacggtcacgcagttcacgcacatacagacgcagctcatgatccaacgtgctggcaccaaatgccgtaaagaagacttccggcattggttcgtgcatcacccttgggtgctcagtcgccgccttcagtaacactttacgcactttttccagatcggagccataggccacgccgagacggatcaccagacgcgtagtagtgtcagtcaacgaccagttgatcagacgctcggtaacaaacgctttgttcgggatgatcacttctttgcgatcgaaatcggtaatcgttgtcgcacgaatacggatcttacttaccgtccccgagaagctaccaatggttaccgtatcgccaatacgcaccggacgttcgaatagaatgatcaaaccggagacgaagttaccgaaaatttcttgtaaaccaaaaccaagacctacggataatgctgcggccagccactggagtttatcccaagagacgcccagcgatccgaacaccgtcatcgcaccaacagcaataatgatgtagttaaggatggtagtaatggcatacgacgcgccctggcgcatattcagtcgcgagagcaccagcacttccagtaaaccaggcaggttgcgaatcaacgcccaggccaccattgaggcgataatcgcaaacaacagactgcccatggtgacgtttttcaccaccgcagcgccagcttcagtgccgttgtaatgccagagcgtgatgctgtcgagatagctgaacacggtgatcaaatcggaccaaattgcccagaacatgacaccgaacagcgcaaacatcagcaacatggtaatacgcagcgtctgctggttaacttgctccagtgcaatggtgggttcttccggcggttcagcaccttctgcgccctctttcaccagattctgccgacgcgccagcgcacgacgccaggcgatacgccgcgccgctacgcttaagccacgcagtaccgtctggtacagcaggttccagatgatcaccagataaacggtttcaatccagcgtcctgccagacgcagcgtagtgtagaagtagcctgtagcagtcagcaccatcagcgcaatcgggattatcgacagcacggtaatggtgaccagtcgcatggtgtgcgactctttatcacgccagctttcgcggcacatcggccataccaggaaggcaatcagcagcaggttgaagaaaatcatcgcttgccccagcacatcatccatcagatgcagcggggaaagttctgccaccacagaccagaaatggataggcagcaatgcgagactgatgcggacaatttgccgacgccagtggctggtctgctgttccggcatgccgaagtgacgtacggcaacgccgtttttctccagtaccttccagcacaggccaaacaccagccagaatatcgccagttttttgctgaacgaccatagcagttcgctgatgttgagctgcatggtcaacagaatcaggccaaccgcgagaataatcaggcacaccggcagcgcacggatcaggtcgataaggatcgcttttggtgtgttgagctggctgtcgttacgcagggaacccacagcggaagccagtttttgttgatacgctttcagccagcccagacgccagtggatcagcccggcaatcaacagcagcggcaaaccagcgaggaaagcgataaaaacggcgggccaggctttttgccagttcaccgtgattttcatcgacttaaattcatctttcaggctttgcgggaacgctttgatccagtcccagtccattggacggttactgttcacccaaaagatttgctgagtcaggatggatttcaggtttttcgacacactcattaactgctgctggttgatttgcaggttaatggccatcatcagctggttacccaactgtttgttgagttgatccagcaattcgcgacgcatatcaaccacttgcaataacgcatcgtgaacttcgctgttgacttcgttggtgtgaccttcttccagtttgttgacgaacgcatcgctctggaagagtgcatcacgctgctggttaacttcaaactgttcgagacgcaaatccgcgatgcggttggtcatgttttccagttcatccgccgagggcagcgtttgttgttgctggtaaaggatacgagacaacagcaggctgcccttcaggacggcaatctgctctttaatattgcgttccgattgcagcgcccgctccagccagtttttgactttaatgttttgctgcatcaactgattaccgttttcagtcgcggtaatcagacgctgacttaactgctggttaatttccagttcctgcttcaccagcggattagcctgaatacgcgcggcttcatccggggagacggcttcctgcgccgttttttcggttaaagtcaggcgcttgctgtttaccgcttcttgcaacagttgtaactggtgctccagacgagcgctgttcgccgtcacgtaatcacgttgcttttgcaaggtatcctgcaagacggtgttcccttccaggcttttacgctgctggtcaatctccgcattcagcaacgcctgctgggcctgcattaacactttctggctgggacgtaaggctgtctcgccgacatcagtcccatccagacgactgcgaatttgttgcagctgctgcgaagcgttatacatcgcattttgcacgcgttcgggctgcgtctgtaacgaaaccagctggctgttataagacgccagatcgttttgtgcgttttgcaaatcgtccagcgcctgggcaacgcgagtttccagctggcgcaacgacagcgtgctcagaattttgcgcgtttcttcgtcgttatcgacatcgctaagtgctgttaacgccgcggtcgcctggcgcattttttccggcgcttcagcgactttttgccgtagctgaactgtctcttcttttatgcgatcgattttatcgagggtggctaatgtatctgtcagatcctgctgcaccagtttgtcctgagcagaaagatctttttgtttatttagtgagtcaagttgcgcctgcaggtccgcttttgtcggcagatcaccattcgatgacgcccgcgcaaacgccgtgttctggcataacaagacaaaaacaaaagcaataaatgctgaaaaaacaaaatgccgtgatcgtttgtaatactggaacatagtcat

SEQ ID NO: 9 - DNA, 3363 bp, synthetic construct
  misc_feature 1..3363: note = Description of artificial sequence: synthetic polynucleotide sequence
  source 1..3363: mol_type = other DNA
atgaccatgttccagtactataagcggtctcgccacttcgtgtttagcgcctttatcgccttcgtgtttgtgctgctgtgccagaacacagccttcgccagggccagctccaatggcgatctgcctaccaaggccgacctgcaggcccagctggattccctgaacaagcagaaggatctgtctgcccaggacaagctggtgcagcaggatctgacagacaccctggccacactggataagatcgaccgcatcaaggaggagaccgtgcagctgaggcagaaggtggcagaggcaccagagaagatgaggcaggcaacagccgccctgaccgccctgagcgacgtggataatgacgaggagacaaggaagatcctgtctaccctgagcctgaggcagctggagacaagggtggcacaggccctggacgatctgcagaacgcccagaatgacctggcctcctacaactctcagctggtgtccctgcagacccagcccgagagagtgcagaacgccatgtataatgccagccagcagctgcagcagatcaggtccagactggatggaacagacgtgggagagaccgccctgaggccttctcagaaggtgctgatgcaggcacagcaggccctgctgaacgcagagatcgatcagcagaggaagagcctggagggcaatacagtgctgcaggacaccctgcagaagcagagagattacgtgacagccaattccgcccggctggagcaccagctgcagctgctgcaggaggccgtgaactccaagagactgacactgaccgagaagaccgcacaggaggccgtgtctccagacgaggccgccagaatccaggccaaccctctggtgaagcaggagctggagatcaatcagcagctgtctcagcggctgatcacagccaccgagaacggcaatcagctgatgcagcagaacatcaaggtgaagaattggctggagcgcgccctgcagagcgagaggaacatcaaggagcagatcgccgtgctgaagggcagcctgctgctgtccagaatcctgtatcagcagcagcagacactgccatccgccgatgagctggagaacatgaccaatagaatcgccgacctgcggctggagcagtttgaggtgaaccagcagcgggacgccctgttccagtctgatgcctttgtgaataagctggaggagggccacaccaacgaagtgaatagcgaggtgcacgacgcactgctgcaggtggtggatatgaggagagagctgctggaccagctgaacaagcagctgggcaatcagctgatgatggccatcaacctgcagatcaatcagcagcagctgatgtctgtgagcaagaatctgaagtctatcctgacacagcagatcttctgggtgaacagcaatagaccaatggattgggactggatcaaggccttcccccagtctctgaaggacgagtttaagagcatgaagatcaccgtgaactggcagaaggcctggcctgccgtgttcatcgcctttctggcaggactgccactgctgctgatcgcaggactgatccactggaggctgggatggctgaaggcataccagcagaagctggcctctgccgtgggcagcctgcgcaacgattctcagctgaatacacccaaggccatcctgatcgacctgatcagggccctgcccgtgtgcctgatcatcctggccgtgggcctgatcctgctgaccatgcagctgaatatctccgagctgctgtggtccttctctaagaagctggccatcttctggctggtgtttggcctgtgctggaaggtgctggagaagaacggcgtggccgtgagacactttggcatgccagagcagcagacaagccactggcggcgccagatcgtgcggatctccctggccctgctgccaatccacttctggtccgtggtggcagagctgtctccactgcacctgatggacgatgtgctgggccaggccatgatcttctttaatctgctgctgatcgcctttctggtgtggcctatgtgcagagagagctggcgggacaaggagtcccacaccatgaggctggtgacaatcaccgtgctgagcatcatcccaatcgccctgatggtgctgacagccaccggctacttctataccacactgcgcctggccggcaggtggatcgagaccgtgtacctggtcatcatctggaacctgctgtatcagaccgtgctgaggggactgtccgtggccgccaggagaatcgcatggaggagggccctggccaggaggcagaatctggtgaaggagggagcagagggagcagagccacctgaggagcccaccatcgccctggagcaggtgaaccagcagacactgaggatcaccatgctgctgatgttcgccctgtttggcgtgatgttctgggccatctggagcgatctgatcacagtgttttcctacctggactctatcacactgtggcactataacggaaccgaggcaggagcagcagtggtgaagaatgtgaccatgggctctctgctgttcgccatcatcgccagcatggtggcctgggccctgatcagaaacctgcctggcctgctggaggtgctggtgctgtccagactgaatatgcggcagggcgcctcttacgccatcaccacaatcctgaactatatcatcatcgccgtgggcgccatgaccgtgtttggcagcctgggcgtgtcctgggataagctgcagtggctggccgccgccctgagcgtgggactgggcttcggcctgcaggagatcttcggcaacttcgtgagcggcctgatcatcctgtttgagcgcccagtgaggatcggcgacacagtgaccatcggcagcttctccggcacagtgagcaagatcaggatcagggccaccacaatcaccgatttcgacagaaaggaagtgatcatccccaacaaggcctttgtgacagagcggctgatcaattggagcctgaccgataccacaaccaggctggtcatcaggctgggagtggcatacggctccgacctggagaaggtgcgcaaggtgctgctgaaggcagcaaccgagcaccctagagtgatgcacgagccaatgcccgaggtgttctttacagcctttggcgcctccaccctggaccacgagctgagactgtatgtgcgggagctgagggacagatctcggacagtggatgagctgaacagaaccatcgatcagctgtgccgggagaacgacatcaatatcgccttcaaccagctggaggtgcacctgcacaatgagaagggcgatgaggtgacagaggtgaagagggactacaagggcgacgatcctaccccagcagtgggatga

SEQ ID NO: 10 - protein, 136 aa, Escherichia coli
MSIIKEFREFAMRGNVVDLAVSVIIGAAFGKIVSSLVADIIMPPLGLLIGGIDFKQFAVTLRDAQGDIPAVVMHYGVFIQNVFDFLIVAFAIFMAIKLINKLNRKKEEPAAAPAPTKEEVLLTEIRDLLKEQNNRS

SEQ ID NO: 11 - DNA, 408 bp, synthetic construct
  misc_feature 1..408: note = Description of artificial sequence: synthetic polynucleotide sequence
  source 1..408: mol_type = other DNA
atgtctatcatcaaggagttccgggagtttgccatgagaggcaacgtggtggacctggccgtgagcgtgatcatcggagcagccttcggcaagatcgtgagcagcctggtggccgacatcatcatgccacctctgggactgctgatcggaggcatcgatttcaagcagtttgcagtgaccctgagggacgcacagggcgacatccctgccgtggtcatgcactacggcgtgtttatccagaacgtgttcgactttctgatcgtggccttcgccatctttatggccatcaagctgatcaacaagctgaataggaagaaggaggagccagctgccgccccagcaccaaccaaggaggaggtgctgctgacagagatcagggatctgctgaaggagcagaacaatcgcagc

SEQ ID NO: 12 - protein, 361 aa, Methanocaldococcus jannaschii
MTITQMISEILMHNTVYNYILSLISIILFIVIGKYANALIERLADKLHKKSGIELDELLIRALSLPVAIAIILSGFYFGVNFLYLLPSLKTAVNEGILTAFILCVVVFFDRFLNELVERYLALTISKKTKKDVDDQIVVLTKKLVRLVVWVVGLLLILSNLGYDIKTLLAGLGIGGLAVALASQNLVSNLIAGLIILTDKPFKIGNWITFSGGSGIVEDIGIRSTKIRATDNSIIVVPNSKLIDEIIQNVPSKNKWKVSTTIGVTYNTPVEKIRKAEEIIKNILLEHPNVEDEPITVYFKEFGDWSLNIQVVYYIKNSRYNGYQKYISTINEVNLKIKEEFDRKGIEFAFPTYTLYLKRDD

SEQ ID NO: 13 - DNA, 1083 bp, Methanocaldococcus jannaschii
  source 1..1083: mol_type = genomic DNA
atgacaataactcaaatgataagtgagattttaatgcacaatactgtatataattatattctgtctctaatctcaattattttatttattgttattgggaaatatgcaaatgcacttatcgaaagacttgcagataaattgcataaaaagagcggtatagaattggatgaacttctaattagggctttatctctacctgtagcaatagcaataatattatctggattttactttggagtaaattttctgtatcttctcccctcattaaaaacagcagtaaatgaagggattttaactgcctttatattgtgtgttgttgtattttttgacagatttcttaatgaacttgtagaaaggtatttagccctaacaatatcaaaaaagacaaaaaaagatgttgatgaccaaattgttgttttaaccaaaaagctcgttagattagttgtgtgggttgttggattgttgttaattttgagcaatcttgggtatgatataaaaactttacttgctggtttgggtattggtggtttagctgtggctttagcgtctcaaaatcttgtttctaatttaatcgctggtttgataattttaactgataaaccatttaagattggaaattggataactttcagtggtggtagtggaattgttgaagatattggaataagaagcacaaagataagggcaacagacaactcaataatcgtagttccaaactcaaaacttatagatgaaattattcaaaatgttccatctaaaaataagtggaaggtttcaacaactataggagtaacttataatacaccagttgaaaaaataaggaaggcagaggaaattataaaaaatatcctcttagaacatccaaatgtagaggatgaaccaataacagtttattttaaggaatttggagattggagtctaaatatccaagtagtttattatattaaaaacagcagatataatggttatcaaaagtatattagcacgataaatgaagttaatttgaaaataaaagaggaatttgatagaaaaggaattgaatttgcattcccaacttatacattatatttaaaaagagacgat

SEQ ID NO: 14 - DNA, 1083 bp, synthetic construct
  misc_feature 1..1083: note = Description of artificial sequence: synthetic polynucleotide sequence
  source 1..1083: mol_type = other DNA
atgaccatcacacagatgatcagcgagatcctgatgcacaacaccgtgtacaattatatcctgagcctgatctccatcatcctgttcatcgtgatcggcaagtacgccaatgccctgatcgagaggctggccgacaagctgcacaagaagtccggcatcgagctggatgagctgctgatccgcgccctgtccctgcctgtggcaatcgccatcatcctgtctggcttctattttggcgtgaactttctgtacctgctgccatctctgaagaccgccgtgaatgagggcatcctgacagccttcatcctgtgcgtggtggtgttctttgacaggtttctgaacgagctggtggagagatacctggccctgaccatctctaagaagacaaagaaggatgtggacgatcagatcgtggtgctgaccaagaagctggtgagactggtggtgtgggtggtgggcctgctgctgatcctgagcaatctgggctacgacatcaagacactgctggccggactgggaatcggaggcctggccgtggccctggcctcccagaacctggtgtctaatctgatcgccggcctgatcatcctgaccgataagcccttcaagatcggcaactggatcacattttctggaggcagcggaatcgtggaggacatcggcatccggagcaccaagatcagagccacagataacagcatcatcgtggtgccaaattccaagctgatcgacgagatcatccagaacgtgccctctaagaataagtggaaggtgagcaccacaatcggcgtgacctataacacacctgtggagaagatccggaaggccgaggagatcatcaagaacatcctgctggagcaccccaatgtggaggacgagcctatcaccgtgtacttcaaggagtttggcgattggtccctgaatatccaggtggtgtactatatcaagaacagccggtacaatggctatcagaagtacatctccacaatcaacgaagtgaatctgaagatcaaggaggagttcgatcggaagggcatcgagttcgcctttcccacctatacactgtacctgaagagagacgat

SEQ ID NO: 15 - protein, 350 aa, Methanocaldococcus jannaschii
MNMEIFGNSISNILIFVVITLLGIFIGKIVDKIVRNYLKKIIDKTKTKFDDIILESIDLPIIVLVVTLFFYFGLRFLILPDYILKLIDEAVKVVVILSATYFAVKFIDGIFEHYLIPLTEKTETELDEHIIKPLKKVVKILTILLGILTALSSVGYDITALLAGLGVGGLALALAMQDTIKNFIAGILILIDKPFSLGHWVKVKGAEGIVEEIGIRSTRIRTFDYTLITIPNSELLDSAIENLTVRDRRRVLMTIGLTYNTPVEKIKRAKEIIKEIVENHPATLPPYRVHFREYGDWSLNLRVEYFVRNMGFDYYLNAVDEINLKIKEEFEKEGIEMAFPTYTVYLEKDN

SEQ ID NO: 16 - DNA, 1050 bp, Methanocaldococcus jannaschii
  source 1..1050: mol_type = genomic DNA
gttatctttctccaaataaacagtatatgttgggaatgccatctctatcccttctttttcaaattcctcttttatcttcaaatttatttcatcaacggcatttaaatagtaatcaaatcccatgtttctaacaaagtattctaccctcaaatttaaactccaatctccatattccctaaaatgcactctatatggagggagagtagctggatgattttcaacaatctcttttattatctccttagccctcttaattttctctaccggtgtgttataagttaaaccgatagtcattaaaacccttcttctatctctaactgttaagttttcaatggctgaatccaacaattctgagtttgggatagttattaaagtgtaatcaaaagttctaattcgtgtgcttcttattccaatctcctctacaatcccttcagcccctttaactttaacccaatggcctaaactaaagggtttgtcaatcaatattaaaatcccagcaatgaagttttttatggtgtcttgcatagccaaagctaaagctaaaccccctactcctaaaccagccaataaagcagtgatatcataaccaacagagcttaaagccgttaatataccaagaagtattgttaatatctttacaacttttttcaatggctttattatgtgttcgtccaactctgtttctgtcttttcggttaatggaattaggtagtgttcaaatatcccatcaataaatttaactgcaaaatatgtagccgataagataactacaacttttactgcttcatctatcaacttgagtatataatctggcagaattaaaaatcttaacccaaaatagaaaaacaatgtaactactaacacaataattggtaaatcaatagactctaatattatatcatcgaattttgtttttgttttatctatgatttttttgagataatttctaactattttatccacaatttttccaatgaaaatacccaatagagttataacaacaaaaataagtatattagatatgctgttcccaaaaatttccatattcac

SEQ ID NO: 17 - DNA, 1050 bp, synthetic construct
  misc_feature 1..1050: note = Description of artificial sequence: synthetic polynucleotide sequence
  source 1..1050: mol_type = other DNA
atgaacatggagatcttcggcaactctatcagcaatatcctgatcttcgtggtcatcaccctgctgggcatctttatcggcaagatcgtggacaagatcgtgcgcaattacctgaagaagatcatcgataagaccaagacaaagtttgacgacatcatcctggagtctatcgacctgcccatcatcgtgctggtggtgacactgttcttttacttcggcctgcggtttctgatcctgcctgactatatcctgaagctgatcgatgaggccgtgaaggtggtggtcatcctgagcgccacctacttcgccgtgaagtttatcgacggcatcttcgagcactatctgatcccactgacagagaagaccgagacagagctggatgagcacatcatcaagcccctgaagaaggtggtgaagatcctgaccatcctgctgggcatcctgaccgccctgagcagcgtgggctatgacatcaccgccctgctggccggactgggagtgggaggcctggccctggccctggccatgcaggacacaatcaagaacttcatcgccggcatcctgatcctgatcgataagcccttcagcctgggccactgggtgaaggtgaagggagcagagggaatcgtggaggagatcggcatcagatccacccgcatccggacattcgattacaccctgatcacaatccccaactccgagctgctggactctgccatcgagaatctgaccgtgagagatcggagaagggtgctgatgacaatcggcctgacctataacacacctgtggagaagatcaagagggccaaggagatcatcaaggagatcgtggagaatcacccagccaccctgcccccttacagagtgcactttagggagtatggcgactggtccctgaacctgcgcgtggagtacttcgtgcggaatatgggctttgactactatctgaacgccgtggatgagatcaatctgaagatcaaggaggagttcgagaaggagggcatcgagatggcctttcctacctacacagtgtatctggagaaggataat

SEQ ID NO: 18 - protein, 678 aa, Arabidopsis thaliana
MMMRTVALPLSHDLNVHKIHEASGFHNSAAGKNRVYLTRTGLSSCATRQDVWSLQLLESLSGSIVPVSSRCNAFVCRSALSPGNGNEGPILKSTAVIFTRVYDALGGNPHLVKLIPAVGILAFATWGLRPLLRLARTTLFEKGNDANSQKSSTQYIVVSYLQPLLLWSGAILLCRTLDPIVLPSSAGQAIKQRLLIFARSISTVLAFSCCLSSLLQQVQKFFMETNNPADTRNMGFSFAGKAVYTAAWVAAASLFMELLGFSTQKWLTAGGLGTVLLTLAGREILTNFLSSIMIHATRPFVLNEWIQTKIGGYEVSGTVEQVGWWSPTIIRGDDREAVHIPNHQFSVNIVRNLTQKTHWRIKTHLAISHLDVSKINNIVADMRKVLSKNPQIEQQKIHRRVFLEDIDPENQALRILISCFVKTSRFEEYLCVKEAVLLDLLTVIRHHGARLATPIRTVQRMRNEAEVDTAGFSDIVFNQAAMNRRYMLIEPSYKINSDDNSKSPSPSPGQKSPSPGQKSEERDLQEEPSETKAETENNGSVPVSNAKKENQKAALGSNSNTGTKGSSTSTSDQPVAQKSEEKKKESVGDPHKAEKDEVSDDEATIEQTLKSKAKQGSEKNNGESKARDGGGSGTSSLLEENLVLGVALDGSKRTLPIDEEHKASGALMDSEELGIGSE

SEQ ID NO: 19 - DNA, 2034 bp, Arabidopsis thaliana
  source 1..2034: mol_type = genomic DNA
atgatgatgcgtactgttgctttaccattgtcccatgatctgaatgttcataagatccatgaagcttctggattccataatagtgctgctggtaaaaatcgtgtgtatctgactcggactggtctttcatcatgtgccacgagacaagacgtttggagtcttcagctactagagagcttgagtggttcaatagtacctgtatcatctaggtgtaatgcatttgtttgccggtcagctctctctcctgggaatggaaatgaaggccctattcttaaatcaactgcagtaatattcacaagggtatatgatgctttaggtggaaatcctcatttagtgaaactaattccagcggttgggattcttgcatttgctacatggggtcttagaccccttctgcgccttgccagaactaccctgtttgagaaggggaatgatgcaaattcacagaagagtagcacgcagtacattgttgtgtcatatcttcaacctttgctgctttggagcggagcaatccttttatgcagaacattggacccaatagtattgccttcaagtgccggccaggctattaaacagcgtcttctgatctttgctcggtccatatcaacggtgttggcattttcctgctgtttatcaagcctacttcagcaggtgcagaaattttttatggagacaaataatcctgctgataccagaaatatgggtttcagttttgctggaaaagctgtttacactgctgcgtgggttgctgctgcttcattgtttatggaactgttaggcttctctacccaaaaatggctaacggctgggggtctggggacagtactgctaactcttgctggccgtgagatacttactaactttctttcaagcattatgattcatgctacacggccctttgttctgaatgagtggatccagaccaagataggaggctatgaagtttctggcacagtagagcaagtcggttggtggtcacctacaattatcagaggtgatgaccgggaagcagttcatattcctaaccaccagttcagtgtgaatattgtgagaaatctcactcagaagacgcattggcgcatcaaaacacatcttgccatcagtcatcttgatgtcagcaaaattaataatatcgtggctgatatgcgcaaggtgttgtctaaaaatcctcaaatcgagcagcaaaaaatacacagaagagtctttctggaggatatagatccagagaaccaagcccttaggattctaatatcctgttttgtcaagacttcacgttttgaagaatacctgtgcgttaaggaagcagtgctcttggatcttcttactgttattaggcatcacggggcacgtctagcaactcccattagaacagtacagaggatgcgtaatgaggctgaggtggacactgcaggattttcagacatcgttttcaatcaagcagcaatgaaccgccgatacatgctgattgagccatcttataaaatcaacagcgatgacaattcaaagtctccctcacccagcccagggcaaaagtcacccagcccagggcaaaagagtgaagaaagggaccttcaagaagaaccatcggaaaccaaggccgaaacagaaaacaacggatcggtgccggtttcaaatgccaaaaaggaaaaccagaaagcggctcttggttccaactccaatacaggcactaagggttcatcaacctcaacatctgaccagccagtagcacaaaagtcagaagaaaagaagaaagagagcgtgggagatccacataaagcagagaaggatgaagtttctgatgacgaagctacaatagagcagacactgaaatcaaaagcaaaacaagggagcgagaaaaacaatggagagtcgaaagctcgagatggtggtgggtccggtactagttctttactggaggagaatcttgttcttggtgttgcgttggatggctcaaaacgtacactcccgattgatgaagaacacaaagcatctggtgcactaatggattcagaagaacttggtattggatcagaa

SEQ ID NO: 20 - DNA, 2034 bp, synthetic construct
  misc_feature 1..2034: note = Description of artificial sequence: synthetic polynucleotide sequence
  source 1..2034: mol_type = other DNA
atgatgatgcggacagtggccctgccactgtcccacgacctgaacgtgcacaagatccacgaggcctctggattccacaacagcgccgcaggcaagaatagggtgtacctgaccaggacaggactgagcagctgcgcaaccagacaggacgtgtggtccctgcagctgctggagtccctgtctggcagcatcgtgcccgtgtctagccggtgcaacgccttcgtgtgcagatccgccctgtctcctggcaacggcaatgagggcccaatcctgaagagcaccgccgtgatctttacaagggtgtatgacgcactgggaggaaatccacacctggtgaagctgatccctgcagtgggcatcctggccttcgccacctggggactgcgccctctgctgaggctggcccgcaccacactgtttgagaagggcaacgacgccaatagccagaagtcctctacacagtacatcgtggtgagctatctgcagccactgctgctgtggtccggagcaatcctgctgtgccggaccctggaccccatcgtgctgcccagctccgccggacaggccatcaagcagaggctgctgatcttcgcccgctccatctctacagtgctggccttttcctgctgtctgtctagcctgctgcagcaggtgcagaagttctttatggagaccaacaatcctgccgatacacgcaacatgggcttctcttttgccggcaaggccgtgtacaccgcagcatgggtggccgccgccagcctgttcatggagctgctgggctttagcacccagaagtggctgacagcaggaggactgggcaccgtgctgctgacactggccggaagggagatcctgacaaacttcctgtcctctatcatgatccacgccacccgcccctttgtgctgaatgagtggattcagaccaagatcggcggctatgaggtgtccggcacagtggagcaagtgggctggtggtctccaaccatcatccggggcgacgatagagaggccgtgcacatccccaatcaccagttctccgtgaacatcgtgaggaatctgacccagaagacacactggcgcatcaagacccacctggccatctcccacctggacgtgtctaagatcaacaatatcgtggccgatatgcggaaggtgctgagcaagaacccacagatcgagcagcagaagatccaccggagagtgtttctggaggacatcgatcccgagaatcaggccctgagaatcctgatcagctgcttcgtgaagacaagcagatttgaggagtacctgtgcgtgaaggaggccgtgctgctggacctgctgaccgtgatcaggcaccacggcgccagactggccaccccaatcaggacagtgcagaggatgagaaacgaggcagaggtggacaccgcaggcttctccgacatcgtgttcaaccaggccgccatgaataggcgctacatgctgatcgagcctagctataagatcaactccgacgataatagcaagtccccctctcctagcccaggccagaagtccccatctcctggccagaagagcgaggagagagacctgcaggaggagccttccgagaccaaggccgagacagagaacaatggcagcgtgccagtgtccaacgccaagaaggagaatcagaaggccgccctgggcagcaactccaataccggcacaaagggcagctccacctctacaagcgatcagcccgtggcccagaagtctgaggagaagaagaaggagagcgtgggcgaccctcacaaggccgagaaggatgaggtgagcgacgatgaggccaccatcgagcagacactgaagtccaaggccaagcagggctctgagaagaacaatggagagtccaaggccagagacggaggaggctctggcacatctagcctgctggaggagaatctggtgctgggagtggccctggacggctctaagagaaccctgcccatcgatgaggagcacaaggccagcggcgccctgatggattccgaggagctgggcatcggcagcgag

SEQ ID NO: 21 - protein, 283 aa, Archaeoglobus fulgidus
MGIMIDVLNYKLYGDVTVYDIIVVIVVMALATIIAKLITTNLRRALIDKMKRDQLELMLKVIYFGIIIVAFIAVLPALGLDLSGLLVAGGITGIVLGFASQSVVANLVSGIFLISEKPIKIGDQVNIDGVAGFVEDVNILSTIIRTYDGLYVRIPNEKVFTSNITNYVAHIARRFEYVVGIRYSDDAEKAIEIIKRIIEEHPFALKNPEPVVFVDNLGDSSVNIVVRIWAPSTEWYNVKMELLWKIKTELEKNGIEIPFPQRVVWFANELRANVEGKEERRQA

SEQ ID NO: 22 - DNA, 849 bp, Archaeoglobus fulgidus
  source 1..849: mol_type = genomic DNA
gtggggattatgattgacgtgctgaattacaagctgtatggagacgtcacggtttacgacattatcgtcgttattgtcgtcatggcccttgccacaataattgcaaagctcataacaaccaacctaaggagggctctgattgacaaaatgaagagggaccagcttgagctgatgctcaaggttatctacttcggaataatcatcgttgccttcatcgcagttttgccggctctgggccttgacctttcaggtttactggttgccggtggaattaccggaattgttctaggttttgccagccagagcgtcgttgcaaacctcgtttcgggaatcttcctgatctcggagaaaccgataaaaattggagatcaggttaacattgacggtgtggcgggcttcgtcgaggatgttaacatcctatcaaccattataagaacctacgatggtttgtacgtcagaatcccaaacgagaaggtcttcacgtccaacataaccaactacgtcgcccacatcgcaaggaggtttgagtacgttgtgggcattaggtacagcgatgatgctgagaaagccatcgagataataaaaagaattattgaggagcaccccttcgccctgaaaaatcccgagcccgtggttttcgttgacaatcttggggatagcagtgtgaacatcgtcgtgagaatctgggctccctcgacggagtggtacaacgtgaagatggagcttctctggaagataaagactgagcttgagaagaacggaatcgagatacctttcccgcagcgtgtggtgtggtttgccaacgagttgagagccaatgttgagggtaaggaagagagacggcaggct

SEQ ID NO: 23 - DNA, 849 bp, synthetic construct
  misc_feature 1..849: note = Description of artificial sequence: synthetic polynucleotide sequence
  source 1..849: mol_type = other DNA
atgggcatcatgatcgacgtgctgaactacaagctgtatggcgacgtgaccgtgtacgacatcatcgtggtcatcgtggtcatggccctggccacaatcatcgccaagctgatcaccacaaatctgcggagagccctgatcgacaagatgaagcgggatcagctggagctgatgctgaaagtgatctacttcggcatcatcatcgtggcctttatcgccgtgctgcccgccctgggcctggatctgtccggactgctggtggcaggaggaatcaccggcatcgtgctgggcttcgcctctcagagcgtggtggccaacctggtgtccggcatctttctgatctctgagaagcccatcaagatcggcgaccaggtgaacatcgatggcgtggccggcttcgtggaggacgtgaatatcctgagcaccatcatcaggacatacgatggcctgtatgtgcgcatccctaatgagaaggtgttcacctccaacatcacaaattacgtggcccacatcgccaggcgctttgagtacgtggtgggcatcaggtattctgacgatgccgagaaggccatcgagatcatcaagcgcatcatcgaggagcaccctttcgccctgaagaaccccgagcctgtggtgtttgtggacaacctgggcgatagctccgtgaatatcgtggtgagaatctgggccccaagcaccgagtggtataacgtgaagatggagctgctgtggaagatcaagacagagctggagaagaatggcatcgagatcccattcccccagcgggtggtgtggtttgccaacgagctgagagccaatgtggagggcaaggaggagcggagacaggcc

SEQ ID NO: 24 - protein, 379 aa, Bacillus halodurans
MNSYYARQWERFVQSFERFNWQDYLIPVAIFIIFLVFRKVFTKYIFKLILRLAKKSKTDVLTNLLLSFEKPLRAFWIILGTYLALMALPFPVTAVAFVDHLYRSLLILLLGWGFFNYTAAHSTIFLTIAKWMDLDENSMVIPFLSKMLRFIVVALTILIILAEWEFKIGGFIAGLGLGGLAFALAAQDTIGNFFGGVIIVTEKPFSKGDWIQTPTVEGVVEDITFRSTRVRTFADSVVTVPNSTLASEPITNWSQMRKRRITFNLGLEYATTKEQLQSVRTKIEAYLRQHDQVDQEVIMVHFSEFNSSSLDIFIYFFTNTIVWSEWYVVKEEINLKIIEILEEEGVSVAFPSRSVYMKRESEGEILPPQKALESKEKDQ

SEQ ID NO: 25 - DNA, 1137 bp, Bacillus halodurans
  source 1..1137: mol_type = genomic DNA
ttggtctttttcttttgattctaacgctttttgcggaggtaagatctcaccctctgattctcgcttcatatacacgctgcggctcgggaacgcaacagatactccttcttcctctaaaatctcgatgatttttaagttgatctcttctttcaccacataccattcagaccagacgatggtgttcgtaaagaaatagataaaaatatcgaggctagaggagttaaattcactaaaatgaaccatgatcacctcttgatccacttgatcgtgctgacgcaaataggcttcaattttcgttcgcaccgattggagctgctctttcgtcgttgcgtattcaagtccaaggttaaacgtaatccggcgttttcgcatctgggaccagttcgtaatcggttcacttgcaagcgttgagttcggaacggttaccaccgaatccgcaaacgtgcgaactctcgtgctacggaacgttatatcttcaaccacaccctcaacagttggcgtctggatccaatcccctttcgagaatggcttttcagtcacgataatgaccccgccgaaaaaattgccgatcgtatcttgtgcggccaaggcaaacgccaatccccctaagcctagacctgcgataaagccaccgatcttaaactcccattccgcaaggatgatcagaatggtgagggccacgacaataaaccgtagcattttggacaaaaacgggataaccatactgttttcatcaagatccatccatttggcaattgttaagaagatcgtcgaatgggccgctgtataattaaaaaatccccatcctaaaagtaaaattaatagcgaacggtacaagtggtctacaaaggcgacagctgtcacggggaacggcaaagccataagagctaagtatgttccgagaataatccaaaacgcccgcaacggcttctcaaaggaaagtaacaagtttgtaaggacgtcggttttcgactttttcgctagccgtaaaataagcttaaaaatatacttcgtaaatacttttcgaaatacaagaaagatgataaaaatcgcaacgggtataagatagtcttgccagttaaaccgttcaaacgattgaacaaaacgttcccattgtctcgcgtaatatgaattcat

SEQ ID NO: 26 - DNA, 1137 bp, synthetic construct
  misc_feature 1..1137: note = Description of artificial sequence: synthetic polynucleotide sequence
  source 1..1137: mol_type = other DNA
atgaacagttactatgcaaggcagtgggagcggtttgtccagtcatttgaacggtttaattggcaggattatctgattccagtggctatcttcatcatctttctggtgttccggaaggtgtttaccaagtacatcttcaagctgatcctgagactggccaagaagtctaagaccgatgtgctgacaaacctgctgctgagctttgagaagcccctgagggccttctggatcatcctgggcacatatctggccctgatggcactgccatttcctgtgaccgcagtggccttcgtggaccacctgtaccgctccctgctgatcctgctgctgggctggggcttctttaactatacagccgcccactctaccatctttctgacaatcgccaagtggatggacctggatgagaacagcatggtgatcccttttctgtctaagatgctgcggttcatcgtggtggccctgaccatcctgatcatcctggccgagtgggagtttaagatcggcggcttcatcgcaggcctgggcctgggaggcctggcctttgccctggccgcccaggatacaatcggcaatttctttggcggcgtgatcatcgtgaccgagaagcctttctccaagggcgactggattcagaccccaacagtggagggcgtggtggaggacatcacctttcggagcacaagggtgcgcaccttcgccgactccgtggtgacagtgccaaactctaccctggccagcgagcccatcacaaattggagccagatgagaaagcggagaatcaccttcaacctgggcctggagtacgccaccacaaaggagcagctgcagagcgtgaggaccaagatcgaggcctatctgcgccagcacgaccaggtggatcaggaggtgatcatggtgcactttagcgagttcaacagctcctctctggacatcttcatctacttctttaccaacacaatcgtgtggtccgagtggtacgtggtgaaggaggagatcaatctgaagatcatcgagatcctggaggaggagggcgtgagcgtggcattccccagccggtccgtgtacatgaagagagaatccgaaggggaaatcctgccacctcagaaagcactggaaagcaaagagaaagatcag

(19) United States
(12) Patent Application Publication
(10) Pub. No.: US 2023/0340408 A1
(43) Pub. Date: Oct. 26, 2023

(54) SONOGENETIC MODULATION OF CELLS EXPRESSING BACTERIALLY-DERIVED MECHANOSENSITIVE PROTEINS

(71) Applicants: Salk Institute for Biological Studies, La Jolla, CA (US); The Regents of the University of California, Oakland, CA (US)

(72) Inventors: Sreekanth CHALASANI, La Jolla, CA (US); Corinne LEE-KUBLI, La Jolla, CA (US); Uri MAGARAM, La Jolla, CA (US); Daniel GIBBS, Oakland, CA (US)

(73) Assignees: Salk Institute for Biological Studies, La Jolla, CA (US); The Regents of the University of California, Oakland, CA (US)

(21) Appl. No.: 18/311,075

(22) Filed: May 2, 2023

Related U.S. Application Data
(63) Continuation of application No. PCT/US2021/057646, filed on Nov. 2, 2021.
(60) Provisional application No. 63/109,578, filed on Nov. 4, 2020.

(51) Int. Cl. C12N 5/0793; C12N 15/86; C07K 14/195; A61P 25/28; C12N 13/00

(52) U.S. Cl. CPC C12N 5/0619; C12N 15/86; C07K 14/195; A61P 25/28; C12N 13/00; C12N 2750/14143

(57) ABSTRACT

Provided and described are bacterial mechanosensory polypeptide and encoding polynucleotide products and compositions thereof, methods of expressing such polypeptides and polynucleotides in a cell type of interest, and methods of inducing and/or modifying the activity or function of various types of cells, including neurons, which express exogenous bacterial mechanosensory polypeptides, using ultrasound.

### CROSS-REFERENCE TO RELATED APPLICATIONS

This application is a continuation under 35 U.S.C. § 111(a) of PCT International Patent Application No. PCT/US2021/057646, filed Nov. 2, 2021, designating the United States and published in English, which claims priority to and benefit of U.S. Provisional Application No. 63/109,578, filed Nov. 4, 2020, the entire contents of each of which are incorporated by reference herein.

### STATEMENT OF RIGHTS TO INVENTIONS MADE UNDER FEDERALLY SPONSORED RESEARCH

This work was supported by Grant Number MH111534 from the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: May 2, 2023; Size: 52,958 bytes; Name: 167776-011502US-Sequence_Listing.xml), and the original TXT format, is herein incorporated by reference in its entirety.

### BACKGROUND

Understanding how neural circuits generate specific behaviors requires an ability to identify the participating neurons, record and perturb their activity patterns. The best-understood motor circuit, the crab stomatogastric ganglion (STG) has benefited from electrophysiological access to well-defined cell types as well as an ability to manipulate them. A number of approaches have been developed for manipulating neuronal activity using light (optogenetics) or small molecules. While these methods have revealed insights into circuit computations in a number of model systems including mice, they are associated with drawbacks, such as the difficulty of delivering a stimulus to the target neurons present in deeper brain regions. Thus, there remains a need for new products and methods for the non-invasive stimulation of target neurons and other cell types.

### SUMMARY

Provided and described herein are compositions featuring bacterial mechanosensory polypeptides and polynucleotides, methods for expressing such polypeptides and polynucleotides in a cell type of interest, and methods for inducing the activation of a heterologous bacterial mechanosensory polypeptide expressed in neurons and other cell types using ultrasound. Also provided are treatment and therapeutic methods involving the use of the bacterial mechanosensory polypeptides heterologously expressed in a cell type of interest, such as a neuronal cell type, to control or modulate the activity of neurons, e.g., in the brain and spinal cord, in a non-invasive manner.

In an aspect, a method of inducing cation influx in a cell is provided, in which the method comprises expressing in the cell a polypeptide selected from MscS, MscL, MscK, MscL G22S, MscMJLR, MscMJ, MscS-Like 3, MscSfam, or MscS-like; wherein the polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and applying ultrasound to the cell, thereby inducing cation influx in the cell. In an embodiment of the method, the cell is sensitized to mechanical deformation or stretch caused by ultrasound. In an embodiment of the method, the application of ultrasound effects a change in mechanosensory polypeptide conductance in the cell and activates or modifies cell activity or function.

In an aspect, a method of rendering a cell responsive to mechanical deformation or stretch caused by ultrasound is provided, in which the method comprises (a) transducing a cell to express a heterologous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; (b) applying ultrasound to the cell; and (c) inducing cation influx in the bacterial mechanosensory polypeptide expressing cell and an alteration in cell activity and/or function following the application of ultrasound, thereby rendering the cell responsive to mechanical deformation or stretch caused by ultrasound.

In another aspect, a method of sensitizing a cell to mechanical deformation or stretch caused by ultrasound and activating and/or modifying activity or function of the cell is provided in which the method comprises (a) transducing a cell to express a heterologous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; applying ultrasound to the bacterial mechanosensory polypeptide-expressing cell; and inducing cation influx in the bacterial mechanosensory polypeptide-expressing cell and an alteration in cell activity and/or function following the application of ultrasound, thereby sensitizing the cell to mechanical deformation or stretch caused by ultrasound and activating and/or modifying cell activity or function.

In an embodiment of the methods of any of the above-delineated aspects and embodiments thereof, the polynucleotide sequence encoding the bacterial mechanosensory polypeptide is codon-optimized for expression in a mammalian or human cell and is non-naturally occurring. In an embodiment of the methods of any of the above-delineated aspects and embodiments thereof, the mechanosensory polypeptide is encoded by a polynucleotide sequence having at least 90% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the methods of any of the above-delineated aspects and embodiments thereof, the mechanosensory polypeptide is encoded by a polynucleotide sequence having at least 95% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the methods of any of the above-delineated aspects and embodiments thereof, the mechanosensory polypeptide is encoded by a polynucleotide sequence having at least 98% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively.

In an embodiment of the methods of any of the above-delineated aspects and embodiments thereof, the heterologous, bacterial mechanosensory polypeptide is expressed in the cell following transduction of the cell by a plasmid or viral vector which contains the polynucleotide sequence encoding polypeptide. In an embodiment, the cell is transduced by a viral vector selected from a lentivirus vector or an adeno-associated virus (AAV) vector. In embodiments of the methods of any of the above-delineated aspects and embodiments thereof, the cell is a mammalian cell or the cell is a human cell. In embodiments of the methods of any of the above-delineated aspects and embodiments thereof, the cell is one or more of a muscle cell, a cardiac muscle cell, an insulin secreting cell, a glial cell, or a neuronal cell. In an embodiment, the cell is a neuronal cell. In an embodiment, neuronal cell is selected from a motor neuron, a sensory neuron, an interneuron, or an Agouti-Related Protein-expression positive (AGRP-^{+ve}) neuron. In an embodiment of the methods of any of the above-delineated aspects and embodiments thereof, the ultrasound has a frequency of about 0.2 MHz to about 20 MHz. In an embodiment of the methods of any of the above-delineated aspects and embodiments thereof, the ultrasound has a focal zone of about 1 cubic millimeter to about 1 cubic centimeter. In an embodiment of the methods of any of the above-delineated aspects and embodiments thereof, the method further comprises contacting the cell with a microbubble prior to applying ultrasound. In an embodiment of the methods of any of the above-delineated aspects and embodiments thereof, the cell is in vitro, ex vivo, or in vivo.

In another aspect, a method of treating a disease or disorder in a subject in need thereof is provided, in which the method comprises (i) expressing in a cell of a mammalian subject a heterologous nucleic acid molecule encoding a mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and (ii) applying ultrasound to the cell, thereby treating the disease or disorder in the subject.

In another aspect, a method of treating a disease or disorder in a mammalian subject in need thereof is provided, in which the method comprises (i) transducing into a cell of the subject a polynucleotide molecule encoding an exogenous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively, respectively; (ii) applying ultrasound to the cell; and (iii) inducing cation influx in the bacterial mechanosensory polypeptide-expressing cell and an alteration in cell activity and/or function following the application of ultrasound, thereby treating the disease or disorder in the subject.

In an embodiment of the above-delineated methods of treatment, the cell is one or more of a muscle cell, a cardiac muscle cell, an insulin secreting cell, a glial cell, or a neuronal cell. In an embodiment, the cell is a neuronal cell. In an embodiment, the neuronal cell is selected from a motor neuron, a sensory neuron, an interneuron, or an Agouti-Related Protein-expression positive (AGRP-^{+ve}) neuron. In an embodiment, the ultrasound is applied to the cell in the hypothalamus of the subject. In embodiments, the disease or disorder is a neurological disease or disorder or a neural circuit disease selected from Parkinson's disease, depression, muscle weakness, muscle atrophy, muscle degeneration obsessive-compulsive disorder, an eating disorder, chronic pain, epilepsy, spinal injury, anxiety, Alzheimer's, post-traumatic stress disorder (PTSD), or cervical spinal cord injury. In other embodiments, the disease or disorder is muscle weakness, muscle atrophy, muscle degeneration, spinal injury, or cervical spinal cord injury. In an embodiment, the disease or disorder is an eating disorder.

In another aspect, a method of modulating neuronal activity or function in a subject in need thereof is provided, in which the method comprises (i) transducing into a neuronal cell of the subject a polynucleotide molecule encoding an exogenous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and (ii) applying ultrasound to the neuronal cell; thereby modulating neuronal activity or function in the subject. In an embodiment of the method, the neuronal cell is selected from a motor neuron, a sensory neuron, an interneuron, or an Agouti-Related Protein-expression positive (AGRP-^{+ve}) neuron.

In another aspect, a method of distal modulation of neuronal activity in a subject in need thereof is provided, in which the method comprises (i) expressing in a neuronal cell of a mammalian subject a heterologous nucleic acid molecule encoding a mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and (ii) applying ultrasound to the neuronal cell at a first site in the subject's nervous system and modulating the activity of neuronal cells at a second site in the subject's nervous system; wherein the second site is distal to the first site. In an embodiment of the method, the neuronal include brain neuron cells, hippocampal neuron cells, or motor neurons. In an embodiment of the method, the first site is the spinal cord and the second site is muscle tissue downstream of the first site. In another embodiment of the method, the first site involves brain neuronal cells in the spinal cord and the second site involves motor neuronal cells in muscle tissue downstream of the first site.

In an embodiment of the above-delineated treatment or therapeutic methods and embodiments thereof, the polynucleotide sequence encoding the bacterial mechanosensory polypeptide is codon-optimized for expression in a mammalian or human cell and is non-naturally occurring. In an embodiment of the above-delineated treatment or therapeutic methods and embodiments thereof, the mechanosensory polypeptide is encoded by a polynucleotide sequence having at least 90% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the above-delineated treatment or therapeutic methods and embodiments thereof, the mechanosensory polypeptide is encoded by a polynucleotide sequence having at least 95% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the above-delineated treatment or therapeutic methods and embodiments thereof, the mechanosensory polypeptide is encoded by a polynucleotide sequence having at least 98% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the above-delineated treatment or therapeutic methods and embodiments thereof, the heterologous, bacterial mechanosensory polypeptide is expressed in the cell following transduction of the cell by a plasmid or viral vector which contains the polynucleotide sequence encoding polypeptide. In an embodiment, the cell is transduced by a viral vector selected from a lentivirus vector or an adenovirus-associated virus (AAV) vector. In an embodiment of the above-delineated treatment or therapeutic methods and embodiments thereof, the cell is a mammalian subject is a human. In an embodiment of the above-delineated treatment or therapeutic methods and embodiments thereof, the ultrasound has a frequency of about 0.2 MHz to about 20 MHz. In an embodiment of the above-delineated treatment or therapeutic methods and embodiments thereof, the ultrasound has a focal zone of about 1 cubic millimeter to about 1 cubic centimeter.

In an embodiment of any one of the above-delineated methods and embodiments thereof, the ultrasound is generated using an opto-acoustic system or transducer. In an embodiment, the ultrasound is generated using a lead zirconate titanate (PZT) transducer. In an embodiment of any one of the above-delineated methods and embodiments thereof, the application of ultrasound to the cell results in a behavioral alteration in the subject.

In another aspect, a plasmid or viral vector comprising a polynucleotide encoding a polypeptide selected from MscS, MscL, MscK, MscL G22S, MscMJLR, MscMJ, MscS-Like 3, MscSfam, or MscS-Like is provided; wherein the polynucleotide is codon-optimized for expression in a mammalian cell and is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment, the polypeptide is encoded by a polynucleotide sequence having at least 90% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment, the polypeptide is encoded by a polynucleotide sequence having at least 95% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment, the polypeptide is encoded by a polynucleotide sequence having at least 98% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively. In an embodiment of the vector is a lentivirus vector or an adeno-associated virus (AAV) vector.

In another aspect, a cell comprising the plasmid or viral vector as delineated above and embodiments thereof is provided. In embodiments, and without limitation, the cell is one or more of a muscle cell, a cardiac muscle cell, an insulin secreting cell, a pancreatic cell, an immune cell, a glial cell, or a neuronal cell. In an embodiment, the cell is a neuronal cell. In an embodiment, the neuronal cell is selected from a motor neuron, a sensory neuron, an interneuron, or an Agouti-Related Protein-expression positive (AGRP-^{+ve}) neuron. In embodiments, the cell is a mammalian cell or the cell is a human cell.

In an embodiment of any one of the above-delineated methods and embodiments thereof, the heterologous or exogenous polypeptide is encoded by a polynucleotide sequence comprising a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively.

In an embodiment of the above-delineated vector and embodiments thereof, the polypeptide is encoded by a polynucleotide sequence comprising a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively.

In an embodiment of the above-delineated cell and embodiments thereof, the polypeptide is encoded by a polynucleotide sequence comprising a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively.

In embodiments of any of the above-delineated aspects and embodiments thereof, the mechanosensory polypeptide is encoded by a polynucleotide sequence comprising or consisting essentially of a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26.

In an embodiment of any one of the above-delineated methods, vector, or cell and embodiments thereof, the mechanosensory polypeptide encoded by the polynucleotide sequence is MscS. In an embodiment of any one of the above-delineated methods, vector, or cell and embodiments thereof, the mechanosensory polypeptide encoded by the polynucleotide sequence is MscL. In an embodiment of any one of the above-delineated methods, vector, or cell and embodiments thereof, the mechanosensory polypeptide encoded by the polynucleotide sequence is MscK. In an embodiment of any one of the above-delineated methods, vector, or cell and embodiments thereof, the mechanosensory polypeptide encoded by the polynucleotide sequence is MscL G22S. In an embodiment of any one of the above-delineated methods, vector, or cell and embodiments thereof, the mechanosensory polypeptide encoded by the polynucleotide sequence is MscMJLR. In an embodiment of any one of the above-delineated methods, vector, or cell and embodiments thereof, the mechanosensory polypeptide encoded by the polynucleotide sequence is MscMJ.

In an embodiment of any one of the above-delineated methods, vector, or cell and embodiments thereof, the mechanosensory polypeptide encoded by the polynucleotide sequence is MscS-Like 3. In an embodiment of any one of the above-delineated methods, vector, or cell and embodiments thereof, the mechanosensory polypeptide encoded by the polynucleotide sequence is MscSfam. In an embodiment of any one of the above-delineated methods, vector, or cell and embodiments thereof, the mechanosensory polypeptide encoded by the polynucleotide sequence is MscS-Like.

Compositions and articles as provided and described herein were isolated or otherwise manufactured in connection with the examples provided herein. Other features and advantages of the description and embodiments herein will be apparent from the detailed description, and from the claims.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of the aspects and embodiments described herein. The following definitions are provided to facilitate understanding of certain terms used frequently herein and are not meant to limit the scope of the present disclosure.

By “bacterial mechanosensory polypeptide” or “bacterial mechanotransduction polypeptide” is meant a polypeptide substantially identical to a mechanosensory polypeptide encoded by a wild type prokaryotic organism. In various embodiments, the mechanosensory polypeptide as described herein is or is derived from MscS (e.g., E. coli MscS), MscL (e.g., E. coli MscL), MscK (e.g., E. coli MscK), MscL G22S (e.g., mutated E. coli MscL channel), MscS-like (e.g., B. halodurans), MscMJ (e.g., M. jannereschi), MscMJLR (e.g., M. jannereschi), MscS-Like 3 (e.g., A. thaliana), MscSfam (A. fulgidus), or a fragment thereof. In some embodiments, the mechanosensory polypeptide is MscS. In some embodiments, a codon-optimized polynucleotide sequence encodes the mechanosensory polypeptide. In an embodiment, a mechanosensory polypeptide or mechanotransduction polypeptide responds to mechanical deflection or stretch when expressed in the membrane of a cell into which it is transfected, transformed, or transduced.

By “MscS polypeptide” is meant a mechanosensory protein capable of conferring ultrasound sensitivity on a cell, e.g., a neuron, and having at least about 85% sequence identity to the MscS sequence provided below, a fragment thereof, or a human ortholog thereof, and having the biological activity described herein. In embodiments, the mechanosensory protein has at least about 85%, at least about 90%, at least about 95%, or at least about 98% sequence identity to the MscS polypeptide sequence provided below, a fragment thereof, or a human ortholog thereof, and has the biological activity described herein. The wild type bacterial small conductance mechanosensitive channel, MscS, is comprised of 286 amino acids and is one of four mechanosensitive channels in E. coli that play a role in osmoregulation. In various embodiments, MscS is responsive to membrane pressures of 5-8 mN/m and does not require any other cellular structures for gating function. In some embodiments, MscS, when in its open conformation, forms a pore about 13 Å in diameter, and conducts at about 0.5 nS of stretch. In some embodiments, the MscS polypeptide is the polypeptide as identified by its Genbank Accession number or a functional fragment, homolog, isoform, or ortholog thereof. In some embodiments, the MscS polypeptide is substantially identical to the protein identified by the Genbank Accession number or a functional variant, isoform, homolog, or ortholog having substantial identity thereto. In some embodiments, the MscS polypeptide is the polypeptide identified by the Genbank Accession number ACI78461.1. In some embodiments, the MscS polypeptide is a functional homolog, isoform, ortholog, or fragment of the polypeptide identified by the Genbank Accession number ACI78461.1. In some embodiments, the MscS polypeptide contains the MscS polypeptide sequence provided immediately below. In some embodiments, the MscS polypeptide consists essentially of the MscS polypeptide sequence provided immediately below.

### MscS Polypeptide Sequence:

(SEQ ID NO: 1)
MEDLNVVDSINGAGSWLVANQALLLSYAVNIVAALAIIIVGLIIARMISN
AVNRLMISRKIDATVADFLSALVRYGIIAFTLIAALGRVGVQTASVIAVL
GAAGLAVGLALQGSLSNLAAGVLLVMFRPFRAGEYVDLGGVAGTVLSVQI
FSTTMRTADGKIIVIPNGKIIAGNIINFSREPVRRNEFIIGVAYDSDIDQ
VKQILTNIIQSEDRILKDREMTVRLNELGASSINFVVRVWSNSGDLQNVY
WDVLERIKREFDAAGISFPYPQMDVNFKRVKEDKAA.

By “MscS polynucleotide” is meant a nucleic acid molecule encoding an MscS polypeptide. In particular embodiments, the codons of the MscS polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscS polynucleotide is provided immediately below. In some embodiments, the MscS polynucleotide is the nucleic acid molecule as identified by its Genbank Accession number or a functional fragment, ortholog, or homolog thereof. In some embodiments, the MscS polynucleotide is substantially identical to the nucleic acid molecule identified by the Genbank Accession number or a functional variant, ortholog, or homolog having substantial identity thereto. In some embodiments, the MscS polynucleotide is the nucleic acid molecule identified by the Genbank Accession number EU895900.1. In some embodiments, the MscS polynucleotide is a functional homolog, isoform, or fragment of the nucleic acid molecule identified by the Genbank Accession number EU895900.1. In some embodiments, the MscS polynucleotide contains the MscS polynucleotide sequence provided immediately below. In some embodiments, the MscS polynucleotide consists essentially of the MscS polynucleotide sequence provided immediately below.

### MscS Polynucleotide Sequence:

(SEQ ID NO: 2)
ATGGAAGATTTGAATGTTGTCGATAGCATAAACGGCGCGGGAAGCTGGCT
GGTAGCTAACCAGGCGCTGCTGCTAAGTTATGCAGTAAACATCGTGGCGG
CACTCGCGATCATCATCGTTGGTTTGATTATCGCGCGGATGATTTCCAAC
GCGGTGAATCGCCTGATGATCTCCCGTAAAATCGATGCCACTGTTGCTGA
TTTTCTTTCTGCATTAGTCCGTTACGGTATTATCGCCTTTACGCTAATCG
CAGCCCTGGGACGCGTGGGTGTACAAACAGCGTCAGTCATTGCTGTACTC
GGTGCCGCAGGCTTAGCTGTTGGTCTGGCTTTGCAGGGGTCACTTTCTAA
CCTGGCCGCTGGCGTGTTACTTGTCATGTTCCGCCCGTTCCGTGCCGGAG
AATATGTTGACCTCGGCGGCGTAGCCGGTACTGTGCTGAGTGTGCAGATT
TTCTCCACCACCATGCGTACTGCAGACGGTAAAATTATCGTTATTCCGAA
CGGTAAAATTATTGCCGGAAATATTATTAACTTCTCCCGCGAGCCAGTTC
GCCGTAACGAATTTATTATTGGCGTGGCGTATGATTCCGATATCGATCAG
GTTAAGCAGATCCTGACCAATATTATCCAGTCTGAAGATCGCATTTTGAA
AGATCGCGAAATGACTGTGCGCCTGAACGAACTTGGTGCATCGTCGATTA
ATTTCGTGGTCCGCGTCTGGAGCAACAGCGGCGATCTGCAAAACGTGTAC
TGGGATGTGCTGGAGCGTATTAAACGTGAATTTGATGCCGCCGGTATCAG
CTTCCCGTACCCGCAAATGGATGTGAACTTTAAGCGGGTGAAAGAAGACA
AAGCTGCG.

In some embodiments, for example, for expression in a mammalian cell, e.g., a human cell, the codon-optimized MscS polynucleotide sequence provided below is used, or a sequence with at least 85% sequence identity thereto is used. In embodiments, a sequence with at least 90%, at least 95%, or at least 98% sequence identity thereto is used. Codon-optimized MscS (also termed Gp155 MscS) polynucleotide sequence:

(SEQ ID NO: 3)
ATGGAGGATCTGAATGTCGTGGACTCTATCAATGGGGCCGGGTCTTGGCT
GGTCGCTAACCAGGCACTGCTGCTGAGCTATGCTGTGAACATTGTGGCCG
CTCTGGCCATCATTATCGTCGGGCTGATTATCGCTCGCATGATCAGTAAC
GCAGTGAATAGACTGATGATTTCAAGGAAGATCGATGCTACCGTGGCAGA
CTTCCTGTCTGCCCTGGTCAGATACGGCATTATCGCTTTTACTCTGATTG
CAGCCCTGGGAAGGGTGGGCGTCCAGACCGCCTCCGTGATCGCTGTCCTG
GGAGCTGCAGGACTGGCAGTGGGCCTGGCCCTGCAGGGGTCTCTGAGTAA
TCTGGCCGCTGGCGTGCTGCTGGTCATGTTCAGGCCCTTTCGCGCAGGGG
AGTATGTGGATCTGGGCGGGGTCGCTGGAACAGTGCTGTCCGTCCAGATT
TTCTCTACCACAATGCGCACTGCCGACGGAAAGATTATCGTGATCCCCAA
CGGAAAAATTATCGCCGGCAACATTATCAATTTCAGCCGAGAGCCTGTGC
GGAGAAATGAGTTCATCATCGGCGTGGCCTACGACAGCGATATTGACCAG
GTGAAGCAGATCCTGACCAACATTATCCAGTCCGAAGATCGCATCCTGAA
AGACCGAGAGATGACAGTGCGGCTGAACGAACTGGGCGCCAGCTCCATCA
ATTTCGTGGTCAGAGTGTGGTCAAACAGCGGGGATCTGCAGAACGTGTAC
TGGGACGTGCTGGAGCGGATCAAGCGGGAATTTGATGCAGCCGGCATCTC
ATTCCCCTACCCACAGATGGATGTCAACTTCAAGAGAGTCAAGGAGGATA
AAGCCGCTTAA.

By “MscL polypeptide” is meant a mechanosensory protein capable of conferring ultrasound sensitivity on a cell, e.g., a neuron, and having at least about 85% sequence identity to the MscL polypeptide sequence provided below, a fragment thereof, or a human ortholog thereof, and having the biological activity described herein. In embodiments, the mechanosensory protein has at least about 85%, at least about 90%, at least about 95%, or at least about 98% sequence identity to the MscL polypeptide sequence provided below, a fragment thereof, or a human ortholog thereof, and has the biological activity described herein. In other embodiments, the polypeptide is the polypeptide as identified by its Genbank Accession number or functional fragment or homolog thereof. In some embodiments, the MscL polypeptide is substantially identical to the protein identified by the Genbank Accession number or a variant or homolog having substantial identity thereto. In some embodiments, the MscL polypeptide is the polypeptide as identified by the Genbank Accession number ACI76971.1. In some embodiments, the MscL polypeptide is a homolog or functional fragment of the polypeptide identified by the Genbank Accession number ACI76971.1. In some embodiments, the MscL polypeptide comprises the MscL polypeptide sequence provided immediately below. In some embodiments, the MscL polypeptide consists essentially of the MscL polypeptide sequence provided immediately below.

### MscL Polypeptide Sequence:

(SEQ ID NO: 4)
MSIIKEFREFAMRGNVVDLAVGVIIGAAFGKIVSSLVADIIMPPLGLLIG
GIDFKQFAVTLRDAQGDIPAVVMHYGVFIQNVFDFLIVAFAIFMAIKLIN
KLNRKKEEPAAAPAPTKEEVLLTEIRDLLKEQNNRS.

By “MscL polynucleotide” is meant a nucleic acid molecule encoding an MscL polypeptide. In particular embodiments, the codons of the MscL polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscL polynucleotide is provided immediately below. In some embodiments, the MscL polynucleotide is the nucleic acid molecule as identified by its Genbank Accession number or a functional fragment or homolog thereof. In some embodiments, the MscL polynucleotide is substantially identical to the nucleic acid molecule identified by the Genbank Accession number or a variant or homolog having substantial identity thereto. In some embodiments, the MscL polynucleotide is the nucleic acid molecule identified by the Genbank Accession number EU894410.1. In some embodiments, the MscL polynucleotide is a homolog or functional fragment of the nucleic acid molecule identified by the Genbank Accession number EU894410.1. In some embodiments, the MscL polynucleotide contains the MscL polynucleotide sequence provided immediately below. In some embodiments, the MscL polynucleotide consists essentially of the MscL polynucleotide sequence provided immediately below.

### MscL Polynucleotide Sequence:

(SEQ ID NO: 5)
ATGAGCATTATTAAAGAATTTCGCGAATTTGCGATGCGCGGGAACGTGGT
GGATTTGGCGGTGGGTGTCATTATCGGTGCGGCATTCGGGAAGATTGTAT
CTTCACTGGTTGCCGATATCATCATGCCACCACTGGGCTTATTAATTGGC
GGGATCGACTTTAAACAGTTTGCTGTCACGCTACGCGATGCGCAGGGGGA
TATCCCTGCTGTTGTGATGCATTACGGTGTCTTCATTCAAAACGTCTTTG
ATTTTCTGATTGTGGCCTTTGCCATCTTTATGGCGATTAAGCTAATCAAC
AAACTGAATCGGAAAAAAGAAGAACCAGCAGCCGCACCAGCACCAACTAA
AGAAGAAGTATTACTGACAGAAATTCGTGATTTGCTGAAAGAGCAGAATA
ACCGCTCTTAA.

In some embodiments, for expression in a mammalian cell, e.g., a human cell, the following codon-optimized MscL polynucleotide sequence is used, or a sequence with at least 85% sequence identity thereto is used. In embodiments, a sequence with at least 90%, at least 95%, or at least 98% sequence identity thereto is used.

### Codon-Optimized MscL (Also Termed Gp154 MscL) Polynucleotide Sequence:

(SEQ ID NO: 6)
ATGTCTATCATCAAGGAGTTCAGGGAGTTTGCCATGAGAGGAAACGTGGT
GGACCTGGCAGTGGGCGTGATCATCGGAGCAGCCTTCGGCAAGATCGTGA
GCTCCCTGGTGGCCGACATCATCATGCCACCTCTGGGACTGCTGATCGGA
GGCATCGATTTCAAGCAGTTTGCAGTGACCCTGAGGGACGCACAGGGCGA
TATCCCTGCAGTGGTCATGCACTACGGCGTGTTTATCCAGAACGTGTTCG
ACTTTCTGATCGTGGCCTTCGCCATCTTTATGGCCATCAAGCTGATCAAC
AAGCTGAATCGGAAGAAGGAGGAGCCAGCAGCAGCACCAGCACCAACCAA
GGAGGAGGTGCTGCTGACAGAGATCCGGGATCTGCTGAAGGAGCAGAACA
ATCGCAGCTGA.

By MscK polypeptide is meant a mechanosensory protein capable of conferring ultrasound sensitivity on a cell, e.g., a neuron, and having at least about 85% sequence identity to the MscK polypeptide sequence provided below, a fragment thereof, or a human ortholog thereof, and having the biological activity described herein. In embodiments, the mechanosensory protein has at least about 85%, at least about 90%, at least about 95%, or at least about 98% sequence identity to the MscK polypeptide sequence provided below, a fragment thereof, or a human ortholog thereof, and has the biological activity described herein. In other embodiments, the polypeptide is the polypeptide as identified by its Genbank Accession number or functional fragment or homolog thereof. In some embodiments, the MscK polypeptide is substantially identical to the protein identified by the Genbank Accession number or a functional variant, ortholog, or homolog having substantial identity thereto. In some embodiments, the MscK polypeptide is the polypeptide identified by the Genbank Accession number QKX92491.1. In some embodiments, the MscK polypeptide is a functional homolog, ortholog, or fragment of the polypeptide identified by the Genbank Accession number QKX92491.1. In some embodiments, the MscK polypeptide contains the MscK polypeptide sequence provided immediately below. In some embodiments, the MscK polypeptide consists essentially of the MscK polypeptide sequence provided immediately below.

### MscK Polypeptide Sequence:

(SEQ ID NO: 7)
MTMFQYYKRSRHFVFSAFIAFVFVLLCQNTAFARASSNGDLPTKADLQAQ
LDSLNKQKDLSAQDKLVQQDLTDTLATLDKIDRIKEETVQLRQKVAEAPE
KMRQATAALTALSDVDNDEETRKILSTLSLRQLETRVAQALDDLQNAQND
LASYNSQLVSLQTQPERVQNAMYNASQQLQQIRSRLDGTDVGETALRPSQ
KVLMQAQQALLNAEIDQQRKSLEGNTVLQDTLQKQRDYVTANSARLEHQL
QLLQEAVNSKRLTLTEKTAQEAVSPDEAARIQANPLVKQELEINQQLSQR
LITATENGNQLMQQNIKVKNWLERALQSERNIKEQIAVLKGSLLLSRILY
QQQQTLPSADELENMTNRIADLRLEQFEVNQQRDALFQSDAFVNKLEEGH
TNEVNSEVHDALLQVVDMRRELLDQLNKQLGNQLMMAINLQINQQQLMSV
SKNLKSILTQQIFWVNSNRPMDWDWIKAFPQSLKDEFKSMKITVNWQKAW
PAVFIAFLAGLPLLLIAGLIHWRLGWLKAYQQKLASAVGSLRNDSQLNTP
KAILIDLIRALPVCLIILAVGLILLTMQLNISELLWSFSKKLAIFWLVFG
LCWKVLEKNGVAVRHFGMPEQQTSHWRRQIVRISLALLPIHFWSVVAELS
PLHLMDDVLGQAMIFFNLLLIAFLVWPMCRESWRDKESHTMRLVTITVLS
IIPIALMVLTATGYFYTTLRLAGRWIETVYLVIIWNLLYQTVLRGLSVAA
RRIAWRRALARRQNLVKEGAEGAEPPEEPTIALEQVNQQTLRITMLLMFA
LFGVMFWAIWSDLITVFSYLDSITLWHYNGTEAGAAVVKNVTMGSLLFAI
IASMVAWALIRNLPGLLEVLVLSRLNMRQGASYAITTILNYIIIAVGAMT
VFGSLGVSWDKLQWLAAALSVGLGFGLQEIFGNFVSGLIILFERPVRIGD
TVTIGSFSGTVSKIRIRATTITDFDRKEVIIPNKAFVTERLINWSLTDTT
TRLVIRLGVAYGSDLEKVRKVLLKAATEHPRVMHEPMPEVFFTAFGASTL
DHELRLYVRELRDRSRTVDELNRTIDQLCRENDINIAFNQLEVHLHNEKG
DEVTEVKRDYKGDDPTPAVG.

By “MscK polynucleotide” is meant a nucleic acid molecule encoding an MscK polypeptide. In particular embodiments, the codons of the MscK polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscK polynucleotide is provided immediately below. In some embodiments, the MscK polynucleotide is the nucleic acid molecule as identified by its Genbank Accession number or functional fragment, ortholog, or homolog thereof. In some embodiments, the MscK polynucleotide is substantially identical to the nucleic acid molecule identified by the Genbank Accession number or a functional variant, ortholog, or homolog having substantial identity thereto. In some embodiments, the MscK polynucleotide comprises or consists of base pairs 3505596 to 3508958 (locus tag: HU676_16905) of the nucleic acid molecule identified by the Genbank Accession number CP055259.1. In some embodiments, the MscK polynucleotide is a functional homolog, ortholog, or fragment of base pairs 3505596 to 3508958 (locus tag: HU676_16905) of the nucleic acid molecule identified by the Genbank Accession number CP055259.1. In some embodiments, the MscK polynucleotide contains the MscK polynucleotide sequence provided immediately below. In some embodiments, the MscK polynucleotide consists essentially of the MscK polynucleotide sequence provided immediately below.

### MscK Polynucleotide Sequence:

(SEQ ID NO: 8)
CCCTACCGCTGGCGTCGGGTCATCGCCTTTGTAGTCGCGTTTTACTTCCG
TCACCTCATCGCCCTTCTCGTTGTGCAGATGCACTTCAAGCTGGTTAAAG
GCAATGTTGATGTCGTTTTCACGGCACAGCTGATCGATAGTACGGTTCAG
CTCATCGACAGTACGACTACGGTCACGCAGTTCACGCACATACAGACGCA
GCTCATGATCCAACGTGCTGGCACCAAATGCCGTAAAGAAGACTTCCGGC
ATTGGTTCGTGCATCACCCTTGGGTGCTCAGTCGCCGCCTTCAGTAACAC
TTTACGCACTTTTTCCAGATCGGAGCCATAGGCCACGCCGAGACGGATCA
CCAGACGCGTAGTAGTGTCAGTCAACGACCAGTTGATCAGACGCTCGGTA
ACAAACGCTTTGTTCGGGATGATCACTTCTTTGCGATCGAAATCGGTAAT
CGTTGTCGCACGAATACGGATCTTACTTACCGTCCCCGAGAAGCTACCAA
TGGTTACCGTATCGCCAATACGCACCGGACGTTCGAATAGAATGATCAAA
CCGGAGACGAAGTTACCGAAAATTTCTTGTAAACCAAAACCAAGACCTAC
GGATAATGCTGCGGCCAGCCACTGGAGTTTATCCCAAGAGACGCCCAGCG
ATCCGAACACCGTCATCGCACCAACAGCAATAATGATGTAGTTAAGGATG
GTAGTAATGGCATACGACGCGCCCTGGCGCATATTCAGTCGCGAGAGCAC
CAGCACTTCCAGTAAACCAGGCAGGTTGCGAATCAACGCCCAGGCCACCA
TTGAGGCGATAATCGCAAACAACAGACTGCCCATGGTGACGTTTTTCACC
ACCGCAGCGCCAGCTTCAGTGCCGTTGTAATGCCAGAGCGTGATGCTGTC
GAGATAGCTGAACACGGTGATCAAATCGGACCAAATTGCCCAGAACATGA
CACCGAACAGCGCAAACATCAGCAACATGGTAATACGCAGCGTCTGCTGG
TTAACTTGCTCCAGTGCAATGGTGGGTTCTTCCGGCGGTTCAGCACCTTC
TGCGCCCTCTTTCACCAGATTCTGCCGACGCGCCAGCGCACGACGCCAGG
CGATACGCCGCGCCGCTACGCTTAAGCCACGCAGTACCGTCTGGTACAGC
AGGTTCCAGATGATCACCAGATAAACGGTTTCAATCCAGCGTCCTGCCAG
ACGCAGCGTAGTGTAGAAGTAGCCTGTAGCAGTCAGCACCATCAGCGCAA
TCGGGATTATCGACAGCACGGTAATGGTGACCAGTCGCATGGTGTGCGAC
TCTTTATCACGCCAGCTTTCGCGGCACATCGGCCATACCAGGAAGGCAAT
CAGCAGCAGGTTGAAGAAAATCATCGCTTGCCCCAGCACATCATCCATCA
GATGCAGCGGGGAAAGTTCTGCCACCACAGACCAGAAATGGATAGGCAGC
AATGCGAGACTGATGCGGACAATTTGCCGACGCCAGTGGCTGGTCTGCTG
TTCCGGCATGCCGAAGTGACGTACGGCAACGCCGTTTTTCTCCAGTACCT
TCCAGCACAGGCCAAACACCAGCCAGAATATCGCCAGTTTTTTGCTGAAC
GACCATAGCAGTTCGCTGATGTTGAGCTGCATGGTCAACAGAATCAGGCC
AACCGCGAGAATAATCAGGCACACCGGCAGCGCACGGATCAGGTCGATAA
GGATCGCTTTTGGTGTGTTGAGCTGGCTGTCGTTACGCAGGGAACCCACA
GCGGAAGCCAGTTTTTGTTGATACGCTTTCAGCCAGCCCAGACGCCAGTG
GATCAGCCCGGCAATCAACAGCAGCGGCAAACCAGCGAGGAAAGCGATAA
AAACGGCGGGCCAGGCTTTTTGCCAGTTCACCGTGATTTTCATCGACTTA
AATTCATCTTTCAGGCTTTGCGGGAACGCTTTGATCCAGTCCCAGTCCAT
TGGACGGTTACTGTTCACCCAAAAGATTTGCTGAGTCAGGATGGATTTCA
GGTTTTTCGACACACTCATTAACTGCTGCTGGTTGATTTGCAGGTTAATG
GCCATCATCAGCTGGTTACCCAACTGTTTGTTGAGTTGATCCAGCAATTC
GCGACGCATATCAACCACTTGCAATAACGCATCGTGAACTTCGCTGTTGA
CTTCGTTGGTGTGACCTTCTTCCAGTTTGTTGACGAACGCATCGCTCTGG
AAGAGTGCATCACGCTGCTGGTTAACTTCAAACTGTTCGAGACGCAAATC
CGCGATGCGGTTGGTCATGTTTTCCAGTTCATCCGCCGAGGGCAGCGTTT
GTTGTTGCTGGTAAAGGATACGAGACAACAGCAGGCTGCCCTTCAGGACG
GCAATCTGCTCTTTAATATTGCGTTCCGATTGCAGCGCCCGCTCCAGCCA
GTTTTTGACTTTAATGTTTTGCTGCATCAACTGATTACCGTTTTCAGTCG
CGGTAATCAGACGCTGACTTAACTGCTGGTTAATTTCCAGTTCCTGCTTC
ACCAGCGGATTAGCCTGAATACGCGCGGCTTCATCCGGGGAGACGGCTTC
CTGCGCCGTTTTTTCGGTTAAAGTCAGGCGCTTGCTGTTTACCGCTTCTT
GCAACAGTTGTAACTGGTGCTCCAGACGAGCGCTGTTCGCCGTCACGTAA
TCACGTTGCTTTTGCAAGGTATCCTGCAAGACGGTGTTCCCTTCCAGGCT
TTTACGCTGCTGGTCAATCTCCGCATTCAGCAACGCCTGCTGGGCCTGCA
TTAACACTTTCTGGCTGGGACGTAAGGCTGTCTCGCCGACATCAGTCCCA
TCCAGACGACTGCGAATTTGTTGCAGCTGCTGCGAAGCGTTATACATCGC
ATTTTGCACGCGTTCGGGCTGCGTCTGTAACGAAACCAGCTGGCTGTTAT
AAGACGCCAGATCGTTTTGTGCGTTTTGCAAATCGTCCAGCGCCTGGGCA
ACGCGAGTTTCCAGCTGGCGCAACGACAGCGTGCTCAGAATTTTGCGCGT
TTCTTCGTCGTTATCGACATCGCTAAGTGCTGTTAACGCCGCGGTCGCCT
GGCGCATTTTTTCCGGCGCTTCAGCGACTTTTTGCCGTAGCTGAACTGTC
TCTTCTTTTATGCGATCGATTTTATCGAGGGTGGCTAATGTATCTGTCAG
ATCCTGCTGCACCAGTTTGTCCTGAGCAGAAAGATCTTTTTGTTTATTTA
GTGAGTCAAGTTGCGCCTGCAGGTCCGCTTTTGTCGGCAGATCACCATTC
GATGACGCCCGCGCAAACGCCGTGTTCTGGCATAACAAGACAAAAACAAA
AGCAATAAATGCTGAAAAAACAAAATGCCGTGATCGTTTGTAATACTGGA
ACATAGTCAT.

In some embodiments, for expression in a mammalian cell, e.g., a human cell, the following codon-optimized MscK polynucleotide sequence is used, or a sequence with at least 85% sequence identity thereto is used. In embodiments, a sequence with at least 90%, at least 95%, or at least 98% sequence identity thereto is used. Codon-optimized MscK (also termed Gp153 MscK) polynucleotide sequence:

(SEQ ID NO: 9)
ATGACCATGTTCCAGTACTATAAGCGGTCTCGCCACTTCGTGTTTAGCGC
CTTTATCGCCTTCGTGTTTGTGCTGCTGTGCCAGAACACAGCCTTCGCCA
GGGCCAGCTCCAATGGCGATCTGCCTACCAAGGCCGACCTGCAGGCCCAG
CTGGATTCCCTGAACAAGCAGAAGGATCTGTCTGCCCAGGACAAGCTGGT
GCAGCAGGATCTGACAGACACCCTGGCCACACTGGATAAGATCGACCGCA
TCAAGGAGGAGACCGTGCAGCTGAGGCAGAAGGTGGCAGAGGCACCAGAG
AAGATGAGGCAGGCAACAGCCGCCCTGACCGCCCTGAGCGACGTGGATAA
TGACGAGGAGACAAGGAAGATCCTGTCTACCCTGAGCCTGAGGCAGCTGG
AGACAAGGGTGGCACAGGCCCTGGACGATCTGCAGAACGCCCAGAATGAC
CTGGCCTCCTACAACTCTCAGCTGGTGTCCCTGCAGACCCAGCCCGAGAG
AGTGCAGAACGCCATGTATAATGCCAGCCAGCAGCTGCAGCAGATCAGGT
CCAGACTGGATGGAACAGACGTGGGAGAGACCGCCCTGAGGCCTTCTCAG
AAGGTGCTGATGCAGGCACAGCAGGCCCTGCTGAACGCAGAGATCGATCA
GCAGAGGAAGAGCCTGGAGGGCAATACAGTGCTGCAGGACACCCTGCAGA
AGCAGAGAGATTACGTGACAGCCAATTCCGCCCGGCTGGAGCACCAGCTG
CAGCTGCTGCAGGAGGCCGTGAACTCCAAGAGACTGACACTGACCGAGAA
GACCGCACAGGAGGCCGTGTCTCCAGACGAGGCCGCCAGAATCCAGGCCA
ACCCTCTGGTGAAGCAGGAGCTGGAGATCAATCAGCAGCTGTCTCAGCGG
CTGATCACAGCCACCGAGAACGGCAATCAGCTGATGCAGCAGAACATCAA
GGTGAAGAATTGGCTGGAGCGCGCCCTGCAGAGCGAGAGGAACATCAAGG
AGCAGATCGCCGTGCTGAAGGGCAGCCTGCTGCTGTCCAGAATCCTGTAT
CAGCAGCAGCAGACACTGCCATCCGCCGATGAGCTGGAGAACATGACCAA
TAGAATCGCCGACCTGCGGCTGGAGCAGTTTGAGGTGAACCAGCAGCGGG
ACGCCCTGTTCCAGTCTGATGCCTTTGTGAATAAGCTGGAGGAGGGCCAC
ACCAACGAAGTGAATAGCGAGGTGCACGACGCACTGCTGCAGGTGGTGGA
TATGAGGAGAGAGCTGCTGGACCAGCTGAACAAGCAGCTGGGCAATCAGC
TGATGATGGCCATCAACCTGCAGATCAATCAGCAGCAGCTGATGTCTGTG
AGCAAGAATCTGAAGTCTATCCTGACACAGCAGATCTTCTGGGTGAACAG
CAATAGACCAATGGATTGGGACTGGATCAAGGCCTTCCCCCAGTCTCTGA
AGGACGAGTTTAAGAGCATGAAGATCACCGTGAACTGGCAGAAGGCCTGG
CCTGCCGTGTTCATCGCCTTTCTGGCAGGACTGCCACTGCTGCTGATCGC
AGGACTGATCCACTGGAGGCTGGGATGGCTGAAGGCATACCAGCAGAAGC
TGGCCTCTGCCGTGGGCAGCCTGCGCAACGATTCTCAGCTGAATACACCC
AAGGCCATCCTGATCGACCTGATCAGGGCCCTGCCCGTGTGCCTGATCAT
CCTGGCCGTGGGCCTGATCCTGCTGACCATGCAGCTGAATATCTCCGAGC
TGCTGTGGTCCTTCTCTAAGAAGCTGGCCATCTTCTGGCTGGTGTTTGGC
CTGTGCTGGAAGGTGCTGGAGAAGAACGGCGTGGCCGTGAGACACTTTGG
CATGCCAGAGCAGCAGACAAGCCACTGGCGGCGCCAGATCGTGCGGATCT
CCCTGGCCCTGCTGCCAATCCACTTCTGGTCCGTGGTGGCAGAGCTGTCT
CCACTGCACCTGATGGACGATGTGCTGGGCCAGGCCATGATCTTCTTTAA
TCTGCTGCTGATCGCCTTTCTGGTGTGGCCTATGTGCAGAGAGAGCTGGC
GGGACAAGGAGTCCCACACCATGAGGCTGGTGACAATCACCGTGCTGAGC
ATCATCCCAATCGCCCTGATGGTGCTGACAGCCACCGGCTACTTCTATAC
CACACTGCGCCTGGCCGGCAGGTGGATCGAGACCGTGTACCTGGTCATCA
TCTGGAACCTGCTGTATCAGACCGTGCTGAGGGGACTGTCCGTGGCCGCC
AGGAGAATCGCATGGAGGAGGGCCCTGGCCAGGAGGCAGAATCTGGTGAA
GGAGGGAGCAGAGGGAGCAGAGCCACCTGAGGAGCCCACCATCGCCCTGG
AGCAGGTGAACCAGCAGACACTGAGGATCACCATGCTGCTGATGTTCGCC
CTGTTTGGCGTGATGTTCTGGGCCATCTGGAGCGATCTGATCACAGTGTT
TTCCTACCTGGACTCTATCACACTGTGGCACTATAACGGAACCGAGGCAG
GAGCAGCAGTGGTGAAGAATGTGACCATGGGCTCTCTGCTGTTCGCCATC
ATCGCCAGCATGGTGGCCTGGGCCCTGATCAGAAACCTGCCTGGCCTGCT
GGAGGTGCTGGTGCTGTCCAGACTGAATATGCGGCAGGGCGCCTCTTACG
CCATCACCACAATCCTGAACTATATCATCATCGCCGTGGGCGCCATGACC
GTGTTTGGCAGCCTGGGCGTGTCCTGGGATAAGCTGCAGTGGCTGGCCGC
CGCCCTGAGCGTGGGACTGGGCTTCGGCCTGCAGGAGATCTTCGGCAACT
TCGTGAGCGGCCTGATCATCCTGTTTGAGCGCCCAGTGAGGATCGGCGAC
ACAGTGACCATCGGCAGCTTCTCCGGCACAGTGAGCAAGATCAGGATCAG
GGCCACCACAATCACCGATTTCGACAGAAAGGAAGTGATCATCCCCAACA
AGGCCTTTGTGACAGAGCGGCTGATCAATTGGAGCCTGACCGATACCACA
ACCAGGCTGGTCATCAGGCTGGGAGTGGCATACGGCTCCGACCTGGAGAA
GGTGCGCAAGGTGCTGCTGAAGGCAGCAACCGAGCACCCTAGAGTGATGC
ACGAGCCAATGCCCGAGGTGTTCTTTACAGCCTTTGGCGCCTCCACCCTG
GACCACGAGCTGAGACTGTATGTGCGGGAGCTGAGGGACAGATCTCGGAC
AGTGGATGAGCTGAACAGAACCATCGATCAGCTGTGCCGGGAGAACGACA
TCAATATCGCCTTCAACCAGCTGGAGGTGCACCTGCACAATGAGAAGGGC
GATGAGGTGACAGAGGTGAAGAGGGACTACAAGGGCGACGATCCTACCCC
AGCAGTGGGATGA.

By “MscL G22S polypeptide” is meant an MscL polypeptide, as defined above, including the mutation G22S. An exemplary MscL G22S polypeptide sequence is provided immediately below.

### MscL G22S Polypeptide Sequence:

(SEQ ID NO: 10)
MSIIKEFREFAMRGNVVDLAVSVIIGAAFGKIVSSLVADIIMPPLGLLIG
GIDFKQFAVTLRDAQGDIPAVVMHYGVFIQNVFDFLIVAFAIFMAIKLIN
KLNRKKEEPAAAPAPTKEEVLLTEIRDLLKEQNNRS.

By “MscL G22S polynucleotide” is meant an MscL polynucleotide, as defined above, encoding a polypeptide comprising the mutation G22S. An exemplary MscL G22S polynucleotide sequence codon-optimized for expression in a mammalian cell, e.g., a human cell, is provided immediately below.

### Codon-Optimized MscL G22S (Also Termed Gp176 MscLG22sK12) Polynucleotide Sequence:

(SEQ ID NO: 11)
ATGTCTATCATCAAGGAGTTCCGGGAGTTTGCCATGAGAGGCAACGTGGT
GGACCTGGCCGTGAGCGTGATCATCGGAGCAGCCTTCGGCAAGATCGTGA
GCAGCCTGGTGGCCGACATCATCATGCCACCTCTGGGACTGCTGATCGGA
GGCATCGATTTCAAGCAGTTTGCAGTGACCCTGAGGGACGCACAGGGCGA
CATCCCTGCCGTGGTCATGCACTACGGCGTGTTTATCCAGAACGTGTTCG
ACTTTCTGATCGTGGCCTTCGCCATCTTTATGGCCATCAAGCTGATCAAC
AAGCTGAATAGGAAGAAGGAGGAGCCAGCTGCCGCCCCAGCACCAACCAA
GGAGGAGGTGCTGCTGACAGAGATCAGGGATCTGCTGAAGGAGCAGAACA
ATCGCAGC.

By “MscMJLR polypeptide” is meant a mechanosensory protein capable of conferring ultrasound sensitivity on a cell, e.g., a neuron, and having at least about 85% sequence identity to the MscMJLR polypeptide sequence provided below, a fragment thereof, or a human ortholog thereof, and having the biological activity described herein. In embodiments, the mechanosensory protein has at least about 90%, at least about 95%, or at least about 98% sequence identity to the MscMJLR polypeptide sequence provided below, a fragment thereof, or a human ortholog thereof, and has the biological activity described herein. In other embodiments, the polypeptide is the polypeptide as identified by its Genbank Accession number or a functional fragment, isoform, ortholog, or homolog thereof. In some embodiments, the MscMJLR polypeptide is substantially identical to the protein identified by the Genbank Accession number or a functional variant, ortholog, or homolog having substantial identity thereto. In some embodiments, the MscMJLR polypeptide is the polypeptide identified by the Genbank Accession number AAB99143.1. In some embodiments, the MscMJLR polypeptide is a homolog or functional fragment of the polypeptide identified by the Genbank Accession number AAB99143.1. In some embodiments, the MscMJLR polypeptide contains the MscMJLR polypeptide sequence provided immediately below. In some embodiments, the MscMJLR polypeptide consists essentially of the MscMJLR polypeptide sequence provided immediately below.

### MscMJLR Polypeptide Sequence:

(SEQ ID NO: 12)
MTITQMISEILMHNTVYNYILSLISIILFIVIGKYANALIERLADKLHKK
SGIELDELLIRALSLPVAIAIILSGFYFGVNFLYLLPSLKTAVNEGILTA
FILCVVVFFDRFLNELVERYLALTISKKTKKDVDDQIVVLTKKLVRLVVW
VVGLLLILSNLGYDIKTLLAGLGIGGLAVALASQNLVSNLIAGLIILTDK
PFKIGNWITFSGGSGIVEDIGIRSTKIRATDNSIIVVPNSKLIDEIIQNV
PSKNKWKVSTTIGVTYNTPVEKIRKAEEIIKNILLEHPNVEDEPITVYFK
EFGDWSLNIQVVYYIKNSRYNGYQKYISTINEVNLKIKEEFDRKGIEFAF
PTYTLYLKRDD.

By “MscMJLR polynucleotide” is meant a nucleic acid molecule encoding an MscMJLR polypeptide. In particular embodiments, the codons of the MscMJLR polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscMJLR polynucleotide is provided immediately below. In some embodiments, the MscMJLR polynucleotide is the nucleic acid molecule identified by its Genbank Accession number or functional fragment, ortholog, or homolog thereof. In some embodiments, the MscMJLR polynucleotide is substantially identical to the polynucleotide identified by the Genbank Accession number or a functional variant, ortholog, or homolog having substantial identity thereto. In some embodiments, the MscMJLR polynucleotide comprises or consists of base pairs 1082519 to 1083604 (locus tag: MJ 1143) of the nucleic acid molecule identified by the Genbank Accession number L77117.1. In some embodiments, the MscMJLR polynucleotide is a functional homolog, ortholog, or fragment of base pairs 1082519 to 1083604 (locus tag: MJ_1143) of the nucleic acid molecule identified by the Genbank Accession number L77117.1. In some embodiments, the MscMJLR polynucleotide contains the MscMJLR polynucleotide sequence provided immediately below. In some embodiments, the MscMJLR polynucleotide consists essentially of the MscMJLR polynucleotide sequence provided immediately below.

### MscMJLR Polynucleotide Sequence:

(SEQ ID NO: 13)
ATGACAATAACTCAAATGATAAGTGAGATTTTAATGCACAATACTGTATA
TAATTATATTCTGTCTCTAATCTCAATTATTTTATTTATTGTTATTGGGA
AATATGCAAATGCACTTATCGAAAGACTTGCAGATAAATTGCATAAAAAG
AGCGGTATAGAATTGGATGAACTTCTAATTAGGGCTTTATCTCTACCTGT
AGCAATAGCAATAATATTATCTGGATTTTACTTTGGAGTAAATTTTCTGT
ATCTTCTCCCCTCATTAAAAACAGCAGTAAATGAAGGGATTTTAACTGCC
TTTATATTGTGTGTTGTTGTATTTTTTGACAGATTTCTTAATGAACTTGT
AGAAAGGTATTTAGCCCTAACAATATCAAAAAAGACAAAAAAAGATGTTG
ATGACCAAATTGTTGTTTTAACCAAAAAGCTCGTTAGATTAGTTGTGTGG
GTTGTTGGATTGTTGTTAATTTTGAGCAATCTTGGGTATGATATAAAAAC
TTTACTTGCTGGTTTGGGTATTGGTGGTTTAGCTGTGGCTTTAGCGTCTC
AAAATCTTGTTTCTAATTTAATCGCTGGTTTGATAATTTTAACTGATAAA
CCATTTAAGATTGGAAATTGGATAACTTTCAGTGGTGGTAGTGGAATTGT
TGAAGATATTGGAATAAGAAGCACAAAGATAAGGGCAACAGACAACTCAA
TAATCGTAGTTCCAAACTCAAAACTTATAGATGAAATTATTCAAAATGTT
CCATCTAAAAATAAGTGGAAGGTTTCAACAACTATAGGAGTAACTTATAA
TACACCAGTTGAAAAAATAAGGAAGGCAGAGGAAATTATAAAAAATATCC
TCTTAGAACATCCAAATGTAGAGGATGAACCAATAACAGTTTATTTTAAG
GAATTTGGAGATTGGAGTCTAAATATCCAAGTAGTTTATTATATTAAAAA
CAGCAGATATAATGGTTATCAAAAGTATATTAGCACGATAAATGAAGTTA
ATTTGAAAATAAAAGAGGAATTTGATAGAAAAGGAATTGAATTTGCATTC
CCAACTTATACATTATATTTAAAAAGAGACGAT.

In some embodiments, for expression in a mammalian cell, e.g., a human cell, the following codon-optimized MscMJLR polynucleotide sequence is used, or a sequence with at least 85% sequence identity thereto is used. In some embodiments, a sequence with at least 90%, at least 95%, or at least 98% sequence identity thereto is used.

### Codon-Optimized MscMJLR (Also Termed Gp179 MscMJLR) Polynucleotide Sequence:

(SEQ ID NO: 14)
ATGACCATCACACAGATGATCAGCGAGATCCTGATGCACAACACCGTGTA
CAATTATATCCTGAGCCTGATCTCCATCATCCTGTTCATCGTGATCGGCA
AGTACGCCAATGCCCTGATCGAGAGGCTGGCCGACAAGCTGCACAAGAAG
TCCGGCATCGAGCTGGATGAGCTGCTGATCCGCGCCCTGTCCCTGCCTGT
GGCAATCGCCATCATCCTGTCTGGCTTCTATTTTGGCGTGAACTTTCTGT
ACCTGCTGCCATCTCTGAAGACCGCCGTGAATGAGGGCATCCTGACAGCC
TTCATCCTGTGCGTGGTGGTGTTCTTTGACAGGTTTCTGAACGAGCTGGT
GGAGAGATACCTGGCCCTGACCATCTCTAAGAAGACAAAGAAGGATGTGG
ACGATCAGATCGTGGTGCTGACCAAGAAGCTGGTGAGACTGGTGGTGTGG
GTGGTGGGCCTGCTGCTGATCCTGAGCAATCTGGGCTACGACATCAAGAC
ACTGCTGGCCGGACTGGGAATCGGAGGCCTGGCCGTGGCCCTGGCCTCCC
AGAACCTGGTGTCTAATCTGATCGCCGGCCTGATCATCCTGACCGATAAG
CCCTTCAAGATCGGCAACTGGATCACATTTTCTGGAGGCAGCGGAATCGT
GGAGGACATCGGCATCCGGAGCACCAAGATCAGAGCCACAGATAACAGCA
TCATCGTGGTGCCAAATTCCAAGCTGATCGACGAGATCATCCAGAACGTG
CCCTCTAAGAATAAGTGGAAGGTGAGCACCACAATCGGCGTGACCTATAA
CACACCTGTGGAGAAGATCCGGAAGGCCGAGGAGATCATCAAGAACATCC
TGCTGGAGCACCCCAATGTGGAGGACGAGCCTATCACCGTGTACTTCAAG
GAGTTTGGCGATTGGTCCCTGAATATCCAGGTGGTGTACTATATCAAGAA
CAGCCGGTACAATGGCTATCAGAAGTACATCTCCACAATCAACGAAGTGA
ATCTGAAGATCAAGGAGGAGTTCGATCGGAAGGGCATCGAGTTCGCCTTT
CCCACCTATACACTGTACCTGAAGAGAGACGAT.

By “MscMJ polypeptide” is meant a mechanosensory protein capable of conferring ultrasound sensitivity on a cell, e.g., a neuron, and having at least about 85% sequence identity to the MscMJ polypeptide sequence provided below, a fragment thereof, or a human ortholog thereof, and having the biological activity described herein. In embodiments, the mechanosensory protein has at least about 85%, at least about 90%, at least about 95%, or at least about 98% sequence identity to the MscMJ polypeptide sequence provided below, a fragment thereof, or a human ortholog thereof, and has the biological activity described herein. In other embodiments, the polypeptide is the polypeptide as identified by its Genbank Accession number or a functional fragment, isoform, ortholog, or homolog thereof. In some embodiments, the MscMJ polypeptide is substantially identical to the protein identified by the Genbank Accession number or a functional variant, ortholog, or homolog having substantial identity thereto. In some embodiments, the MscMJ polypeptide is the polypeptide as identified by the Genbank Accession number AAB98155.1. In some embodiments, the MscMJ polypeptide is a functional homolog, ortholog, or fragment of the polypeptide identified by the Genbank Accession number AAB98155.1. In some embodiments, the MscMJ polypeptide contains the MscMJ polypeptide sequence provided immediately below. In some embodiments, the MscMJ polypeptide consists essentially of the MscMJ polypeptide sequence provided immediately below.

### MscMJ Polypeptide Sequence:

(SEQ ID NO: 15)
MNMEIFGNSISNILIFVVITLLGIFIGKIVDKIVRNYLKKIIDKTKTKFD
DIILESIDLPIIVLVVTLFFYFGLRFLILPDYILKLIDEAVKVVVILSAT
YFAVKFIDGIFEHYLIPLTEKTETELDEHIIKPLKKVVKILTILLGILTA
LSSVGYDITALLAGLGVGGLALALAMQDTIKNFIAGILILIDKPFSLGHW
VKVKGAEGIVEEIGIRSTRIRTFDYTLITIPNSELLDSAIENLTVRDRRR
VLMTIGLTYNTPVEKIKRAKEIIKEIVENHPATLPPYRVHFREYGDWSLN
LRVEYFVRNMGFDYYLNAVDEINLKIKEEFEKEGIEMAFPTYTVYLEKD
N.

By “MscMJ polynucleotide” is meant a nucleic acid molecule encoding an MscMJ polypeptide. In particular embodiments, the codons of the MscMJ polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscMJ polynucleotide is provided immediately below. In some embodiments, the MscMJ polynucleotide is the nucleic acid molecule identified by its Genbank Accession number or a functional fragment, ortholog, or homolog thereof. In some embodiments, the MscMJ polynucleotide is substantially identical to the nucleic acid molecule identified by the Genbank Accession number or a variant or homolog having substantial identity thereto. In some embodiments, the MscMJ polynucleotide comprises or consists of base pairs 172170 to 173222 (locus tag: MJ_0170) of the nucleic acid molecule identified by the Genbank Accession number L77117.1. In some embodiments, the MscMJ polynucleotide is a functional homolog, ortholog, or fragment of base pairs 172170 to 173222 (locus tag: MJ_0170) of the nucleic acid molecule identified by the Genbank Accession number L77117.1. In some embodiments, the MscMJ polynucleotide contains the MscMJ polynucleotide sequence provided immediately below. In some embodiments, the MscMJ polynucleotide consists essentially of the MscMJ polynucleotide sequence provided immediately below.

### MscMJ Polynucleotide Sequence:

(SEQ ID NO: 16)
GTTATCTTTCTCCAAATAAACAGTATATGTTGGGAATGCCATCTCTATCC
CTTCTTTTTCAAATTCCTCTTTTATCTTCAAATTTATTTCATCAACGGCA
TTTAAATAGTAATCAAATCCCATGTTTCTAACAAAGTATTCTACCCTCAA
ATTTAAACTCCAATCTCCATATTCCCTAAAATGCACTCTATATGGAGGGA
GAGTAGCTGGATGATTTTCAACAATCTCTTTTATTATCTCCTTAGCCCTC
TTAATTTTCTCTACCGGTGTGTTATAAGTTAAACCGATAGTCATTAAAAC
CCTTCTTCTATCTCTAACTGTTAAGTTTTCAATGGCTGAATCCAACAATT
CTGAGTTTGGGATAGTTATTAAAGTGTAATCAAAAGTTCTAATTCGTGTG
CTTCTTATTCCAATCTCCTCTACAATCCCTTCAGCCCCTTTAACTTTAAC
CCAATGGCCTAAACTAAAGGGTTTGTCAATCAATATTAAAATCCCAGCAA
TGAAGTTTTTTATGGTGTCTTGCATAGCCAAAGCTAAAGCTAAACCCCCT
ACTCCTAAACCAGCCAATAAAGCAGTGATATCATAACCAACAGAGCTTAA
AGCCGTTAATATACCAAGAAGTATTGTTAATATCTTTACAACTTTTTTCA
ATGGCTTTATTATGTGTTCGTCCAACTCTGTTTCTGTCTTTTCGGTTAAT
GGAATTAGGTAGTGTTCAAATATCCCATCAATAAATTTAACTGCAAAATA
TGTAGCCGATAAGATAACTACAACTTTTACTGCTTCATCTATCAACTTGA
GTATATAATCTGGCAGAATTAAAAATCTTAACCCAAAATAGAAAAACAAT
GTAACTACTAACACAATAATTGGTAAATCAATAGACTCTAATATTATATC
ATCGAATTTTGTTTTTGTTTTATCTATGATTTTTTTGAGATAATTTCTAA
CTATTTTATCCACAATTTTTCCAATGAAAATACCCAATAGAGTTATAACA
ACAAAAATAAGTATATTAGATATGCTGTTCCCAAAAATTTCCATATTCA
C.

In some embodiments, for expression in a mammalian cell, e.g., a human cell, the following codon-optimized MscMJ polynucleotide sequence is used, or a sequence with at least 85% sequence identity thereto is used. In some embodiments, a sequence with at least 90%, at least 95%, or at least 98% sequence identity thereto is used.

### Codon-Optimized MscMJ (Also Termed Gp181 MscMJ) Polynucleotide Sequence:

(SEQ ID NO: 17)
ATGAACATGGAGATCTTCGGCAACTCTATCAGCAATATCCTGATCTTCGT
GGTCATCACCCTGCTGGGCATCTTTATCGGCAAGATCGTGGACAAGATCG
TGCGCAATTACCTGAAGAAGATCATCGATAAGACCAAGACAAAGTTTGAC
GACATCATCCTGGAGTCTATCGACCTGCCCATCATCGTGCTGGTGGTGAC
ACTGTTCTTTTACTTCGGCCTGCGGTTTCTGATCCTGCCTGACTATATCC
TGAAGCTGATCGATGAGGCCGTGAAGGTGGTGGTCATCCTGAGCGCCACC
TACTTCGCCGTGAAGTTTATCGACGGCATCTTCGAGCACTATCTGATCCC
ACTGACAGAGAAGACCGAGACAGAGCTGGATGAGCACATCATCAAGCCCC
TGAAGAAGGTGGTGAAGATCCTGACCATCCTGCTGGGCATCCTGACCGCC
CTGAGCAGCGTGGGCTATGACATCACCGCCCTGCTGGCCGGACTGGGAGT
GGGAGGCCTGGCCCTGGCCCTGGCCATGCAGGACACAATCAAGAACTTCA
TCGCCGGCATCCTGATCCTGATCGATAAGCCCTTCAGCCTGGGCCACTGG
GTGAAGGTGAAGGGAGCAGAGGGAATCGTGGAGGAGATCGGCATCAGATC
CACCCGCATCCGGACATTCGATTACACCCTGATCACAATCCCCAACTCCG
AGCTGCTGGACTCTGCCATCGAGAATCTGACCGTGAGAGATCGGAGAAGG
GTGCTGATGACAATCGGCCTGACCTATAACACACCTGTGGAGAAGATCAA
GAGGGCCAAGGAGATCATCAAGGAGATCGTGGAGAATCACCCAGCCACCC
TGCCCCCTTACAGAGTGCACTTTAGGGAGTATGGCGACTGGTCCCTGAAC
CTGCGCGTGGAGTACTTCGTGCGGAATATGGGCTTTGACTACTATCTGAA
CGCCGTGGATGAGATCAATCTGAAGATCAAGGAGGAGTTCGAGAAGGAGG
GCATCGAGATGGCCTTTCCTACCTACACAGTGTATCTGGAGAAGGATAA
T.

By “MscS-Like 3 polypeptide” or “MscS-like 3” is meant a mechanosensory protein, e.g., derived from Arabidopsis thaliana, that is capable of conferring ultrasound sensitivity on a cell, e.g., a neuron, and having at least about 85% sequence identity to the MscS-Like 3 polypeptide sequence provided below, a fragment thereof, or a human ortholog thereof, and having the biological activity described herein. In embodiments, the mechanosensory protein has at least about 85%, at least about 90%, at least about 95%, or at least about 98% sequence identity to the MscS-Like 3 polypeptide sequence provided below, a fragment thereof, or a human ortholog thereof, and has the biological activity described herein. In other embodiments, the polypeptide is the polypeptide as identified by its Genbank Accession number or a functional fragment, isoform, ortholog, or homolog thereof. In some embodiments, the MscS-Like 3 polypeptide is substantially identical to the protein identified by the Genbank Accession number or a functional variant, ortholog, or homolog having substantial identity thereto. In some embodiments, the MscS-Like 3 polypeptide is the polypeptide as identified by the Genbank Accession number AEE33512.1. In some embodiments, the MscS-Like 3 polypeptide is a functional homolog, ortholog, or fragment of the polypeptide identified by the Genbank Accession number AEE33512.1. In some embodiments, the MscS-Like 3 polypeptide contains the MscS-Like 3 polypeptide sequence provided immediately below. In some embodiments, the MscS-Like 3 polypeptide consists essentially of the MscS-Like 3 polypeptide sequence provided immediately below.

### MscS-Like 3 Amino Acid Sequence:

(SEQ ID NO: 18)
MMMRTVALPLSHDLNVHKIHEASGFHNSAAGKNRVYLTRTGLSSCATRQD
VWSLQLLESLSGSIVPVSSRCNAFVCRSALSPGNGNEGPILKSTAVIFTR
VYDALGGNPHLVKLIPAVGILAFATWGLRPLLRLARTTLFEKGNDANSQK
SSTQYIVVSYLQPLLLWSGAILLCRTLDPIVLPSSAGQAIKQRLLIFARS
ISTVLAFSCCLSSLLQQVQKFFMETNNPADTRNMGFSFAGKAVYTAAWVA
AASLFMELLGFSTQKWLTAGGLGTVLLTLAGREILTNFLSSIMIHATRPF
VLNEWIQTKIGGYEVSGTVEQVGWWSPTIIRGDDREAVHIPNHQFSVNIV
RNLTQKTHWRIKTHLAISHLDVSKINNIVADMRKVLSKNPQIEQQKIHRR
VFLEDIDPENQALRILISCFVKTSRFEEYLCVKEAVLLDLLTVIRHHGAR
LATPIRTVQRMRNEAEVDTAGFSDIVFNQAAMNRRYMLIEPSYKINSDDN
SKSPSPSPGQKSPSPGQKSEERDLQEEPSETKAETENNGSVPVSNAKKEN
QKAALGSNSNTGTKGSSTSTSDQPVAQKSEEKKKESVGDPHKAEKDEVSD
DEATIEQTLKSKAKQGSEKNNGESKARDGGGSGTSSLLEENLVLGVALDG
SKRTLPIDEEHKASGALMDSEELGIGSE.

By “MscS-Like 3 polynucleotide” is meant a nucleic acid molecule encoding a MscS-Like 3 polypeptide. In particular embodiments, the codons of the MscS-Like 3 polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscS-Like 3 polynucleotide is provided immediately below. In some embodiments, the nucleic acid molecule is the MscS-Like 3 polynucleotide identified by its Genbank Accession number or a functional fragment, ortholog, or homolog thereof. In some embodiments, the MscS-Like 3 polynucleotide is substantially identical to the nucleic acid molecule identified by the Genbank Accession number or a variant, ortholog or homolog having substantial identity thereto. In some embodiments, the MscS-Like 3 polynucleotide is the nucleic acid molecule identified by the Genbank Accession number NM 202317.3. In some embodiments, the MscS-Like 3 polynucleotide is a functional homolog, ortholog, or fragment of the nucleic acid molecule identified by the Genbank Accession number NM 202317.3. In some embodiments, the MscS-Like 3 polynucleotide contains the MscS-Like 3 polynucleotide sequence provided immediately below. In some embodiments, the MscS-Like 3 polynucleotide consists of the MscS-Like 3 polynucleotide sequence provided immediately below.

### MscS-Like 3 Polynucleotide Sequence:

(SEQ ID NO: 19)
ATGATGATGCGTACTGTTGCTTTACCATTGTCCCATGATCTGAATGTTCA
TAAGATCCATGAAGCTTCTGGATTCCATAATAGTGCTGCTGGTAAAAATC
GTGTGTATCTGACTCGGACTGGTCTTTCATCATGTGCCACGAGACAAGAC
GTTTGGAGTCTTCAGCTACTAGAGAGCTTGAGTGGTTCAATAGTACCTGT
ATCATCTAGGTGTAATGCATTTGTTTGCCGGTCAGCTCTCTCTCCTGGGA
ATGGAAATGAAGGCCCTATTCTTAAATCAACTGCAGTAATATTCACAAGG
GTATATGATGCTTTAGGTGGAAATCCTCATTTAGTGAAACTAATTCCAGC
GGTTGGGATTCTTGCATTTGCTACATGGGGTCTTAGACCCCTTCTGCGCC
TTGCCAGAACTACCCTGTTTGAGAAGGGGAATGATGCAAATTCACAGAAG
AGTAGCACGCAGTACATTGTTGTGTCATATCTTCAACCTTTGCTGCTTTG
GAGCGGAGCAATCCTTTTATGCAGAACATTGGACCCAATAGTATTGCCTT
CAAGTGCCGGCCAGGCTATTAAACAGCGTCTTCTGATCTTTGCTCGGTCC
ATATCAACGGTGTTGGCATTTTCCTGCTGTTTATCAAGCCTACTTCAGCA
GGTGCAGAAATTTTTTATGGAGACAAATAATCCTGCTGATACCAGAAATA
TGGGTTTCAGTTTTGCTGGAAAAGCTGTTTACACTGCTGCGTGGGTTGCT
GCTGCTTCATTGTTTATGGAACTGTTAGGCTTCTCTACCCAAAAATGGCT
AACGGCTGGGGGTCTGGGGACAGTACTGCTAACTCTTGCTGGCCGTGAGA
TACTTACTAACTTTCTTTCAAGCATTATGATTCATGCTACACGGCCCTTT
GTTCTGAATGAGTGGATCCAGACCAAGATAGGAGGCTATGAAGTTTCTGG
CACAGTAGAGCAAGTCGGTTGGTGGTCACCTACAATTATCAGAGGTGATG
ACCGGGAAGCAGTTCATATTCCTAACCACCAGTTCAGTGTGAATATTGTG
AGAAATCTCACTCAGAAGACGCATTGGCGCATCAAAACACATCTTGCCAT
CAGTCATCTTGATGTCAGCAAAATTAATAATATCGTGGCTGATATGCGCA
AGGTGTTGTCTAAAAATCCTCAAATCGAGCAGCAAAAAATACACAGAAGA
GTCTTTCTGGAGGATATAGATCCAGAGAACCAAGCCCTTAGGATTCTAAT
ATCCTGTTTTGTCAAGACTTCACGTTTTGAAGAATACCTGTGCGTTAAGG
AAGCAGTGCTCTTGGATCTTCTTACTGTTATTAGGCATCACGGGGCACGT
CTAGCAACTCCCATTAGAACAGTACAGAGGATGCGTAATGAGGCTGAGGT
GGACACTGCAGGATTTTCAGACATCGTTTTCAATCAAGCAGCAATGAACC
GCCGATACATGCTGATTGAGCCATCTTATAAAATCAACAGCGATGACAAT
TCAAAGTCTCCCTCACCCAGCCCAGGGCAAAAGTCACCCAGCCCAGGGCA
AAAGAGTGAAGAAAGGGACCTTCAAGAAGAACCATCGGAAACCAAGGCCG
AAACAGAAAACAACGGATCGGTGCCGGTTTCAAATGCCAAAAAGGAAAAC
CAGAAAGCGGCTCTTGGTTCCAACTCCAATACAGGCACTAAGGGTTCATC
AACCTCAACATCTGACCAGCCAGTAGCACAAAAGTCAGAAGAAAAGAAGA
AAGAGAGCGTGGGAGATCCACATAAAGCAGAGAAGGATGAAGTTTCTGAT
GACGAAGCTACAATAGAGCAGACACTGAAATCAAAAGCAAAACAAGGGAG
CGAGAAAAACAATGGAGAGTCGAAAGCTCGAGATGGTGGTGGGTCCGGTA
CTAGTTCTTTACTGGAGGAGAATCTTGTTCTTGGTGTTGCGTTGGATGGC
TCAAAACGTACACTCCCGATTGATGAAGAACACAAAGCATCTGGTGCACT
AATGGATTCAGAAGAACTTGGTATTGGATCAGAA.

In some embodiments, for expression in a mammalian cell, e.g., a human cell, the following codon-optimized MscS-Like 3 polynucleotide sequence is used, or a sequence with at least 85% sequence identity thereto is used. In some embodiments, a sequence with at least 90%, at least 95%, or at least 98% sequence identity thereto is used.

Codon-Optimized MscS-Like 3 (Also Termed Gp180 MscLike A. thaliana) Polynucleotide Sequence:

(SEQ ID NO: 20)
ATGATGATGCGGACAGTGGCCCTGCCACTGTCCCACGACCTGAACGTGCA
CAAGATCCACGAGGCCTCTGGATTCCACAACAGCGCCGCAGGCAAGAATA
GGGTGTACCTGACCAGGACAGGACTGAGCAGCTGCGCAACCAGACAGGAC
GTGTGGTCCCTGCAGCTGCTGGAGTCCCTGTCTGGCAGCATCGTGCCCGT
GTCTAGCCGGTGCAACGCCTTCGTGTGCAGATCCGCCCTGTCTCCTGGCA
ACGGCAATGAGGGCCCAATCCTGAAGAGCACCGCCGTGATCTTTACAAGG
GTGTATGACGCACTGGGAGGAAATCCACACCTGGTGAAGCTGATCCCTGC
AGTGGGCATCCTGGCCTTCGCCACCTGGGGACTGCGCCCTCTGCTGAGGC
TGGCCCGCACCACACTGTTTGAGAAGGGCAACGACGCCAATAGCCAGAAG
TCCTCTACACAGTACATCGTGGTGAGCTATCTGCAGCCACTGCTGCTGTG
GTCCGGAGCAATCCTGCTGTGCCGGACCCTGGACCCCATCGTGCTGCCCA
GCTCCGCCGGACAGGCCATCAAGCAGAGGCTGCTGATCTTCGCCCGCTCC
ATCTCTACAGTGCTGGCCTTTTCCTGCTGTCTGTCTAGCCTGCTGCAGCA
GGTGCAGAAGTTCTTTATGGAGACCAACAATCCTGCCGATACACGCAACA
TGGGCTTCTCTTTTGCCGGCAAGGCCGTGTACACCGCAGCATGGGTGGCC
GCCGCCAGCCTGTTCATGGAGCTGCTGGGCTTTAGCACCCAGAAGTGGCT
GACAGCAGGAGGACTGGGCACCGTGCTGCTGACACTGGCCGGAAGGGAGA
TCCTGACAAACTTCCTGTCCTCTATCATGATCCACGCCACCCGCCCCTTT
GTGCTGAATGAGTGGATTCAGACCAAGATCGGCGGCTATGAGGTGTCCGG
CACAGTGGAGCAAGTGGGCTGGTGGTCTCCAACCATCATCCGGGGCGACG
ATAGAGAGGCCGTGCACATCCCCAATCACCAGTTCTCCGTGAACATCGTG
AGGAATCTGACCCAGAAGACACACTGGCGCATCAAGACCCACCTGGCCAT
CTCCCACCTGGACGTGTCTAAGATCAACAATATCGTGGCCGATATGCGGA
AGGTGCTGAGCAAGAACCCACAGATCGAGCAGCAGAAGATCCACCGGAGA
GTGTTTCTGGAGGACATCGATCCCGAGAATCAGGCCCTGAGAATCCTGAT
CAGCTGCTTCGTGAAGACAAGCAGATTTGAGGAGTACCTGTGCGTGAAGG
AGGCCGTGCTGCTGGACCTGCTGACCGTGATCAGGCACCACGGCGCCAGA
CTGGCCACCCCAATCAGGACAGTGCAGAGGATGAGAAACGAGGCAGAGGT
GGACACCGCAGGCTTCTCCGACATCGTGTTCAACCAGGCCGCCATGAATA
GGCGCTACATGCTGATCGAGCCTAGCTATAAGATCAACTCCGACGATAAT
AGCAAGTCCCCCTCTCCTAGCCCAGGCCAGAAGTCCCCATCTCCTGGCCA
GAAGAGCGAGGAGAGAGACCTGCAGGAGGAGCCTTCCGAGACCAAGGCCG
AGACAGAGAACAATGGCAGCGTGCCAGTGTCCAACGCCAAGAAGGAGAAT
CAGAAGGCCGCCCTGGGCAGCAACTCCAATACCGGCACAAAGGGCAGCTC
CACCTCTACAAGCGATCAGCCCGTGGCCCAGAAGTCTGAGGAGAAGAAGA
AGGAGAGCGTGGGCGACCCTCACAAGGCCGAGAAGGATGAGGTGAGCGAC
GATGAGGCCACCATCGAGCAGACACTGAAGTCCAAGGCCAAGCAGGGCTC
TGAGAAGAACAATGGAGAGTCCAAGGCCAGAGACGGAGGAGGCTCTGGCA
CATCTAGCCTGCTGGAGGAGAATCTGGTGCTGGGAGTGGCCCTGGACGGC
TCTAAGAGAACCCTGCCCATCGATGAGGAGCACAAGGCCAGCGGCGCCCT
GATGGATTCCGAGGAGCTGGGCATCGGCAGCGAG.

By “MscSfam polypeptide” is meant a mechanosensory protein derived from Archaeoglobus fulgidus dsm 4304 that is capable of conferring ultrasound sensitivity on a cell, e.g., a neuron, and having at least about 85% sequence identity to the MscSfam polypeptide sequence provided below, a fragment thereof, or a human ortholog thereof, and having the biological activity described herein. In embodiments, the mechanosensory protein has at least about 90%, at least about 95%, or at least about 98% sequence identity to the MscSfam polypeptide sequence provided below, a fragment thereof, or a human ortholog thereof, and has the biological activity described herein. In other embodiments, the polypeptide is the polypeptide as identified by its Genbank Accession number or a functional fragment, isoform, ortholog, or homolog thereof. In some embodiments, the MscSfam polypeptide is substantially identical to the protein identified by the Genbank Accession number or a variant, ortholog, or homolog having substantial identity thereto. In some embodiments, the MscSfam polypeptide is the polypeptide identified by the Genbank Accession number AAB89702.1. In some embodiments, the MscSfam polypeptide is a homolog or functional fragment of the polypeptide identified by the Genbank Accession number AAB89702.1. In some embodiments, the MscSfam polypeptide contains the MscSfam polypeptide sequence provided immediately below. In some embodiments, the MscSfam polypeptide consists essentially of the MscSfam polypeptide sequence provided immediately below.

### MscSfam Polypeptide Sequence:

(SEQ ID NO: 21)
MGIMIDVLNYKLYGDVTVYDIIVVIVVMALATIIAKLITTNLRRALIDKM
KRDQLELMLKVIYFGIIIVAFIAVLPALGLDLSGLLVAGGITGIVLGFAS
QSVVANLVSGIFLISEKPIKIGDQVNIDGVAGFVEDVNILSTHIRTYDGL
YVRIPNEKVFTSNITNYVAHIARRFEYVVGIRYSDDAEKAIEIIKRIIEE
HPFALKNPEPVVFVDNLGDSSVNIVVRIWAPSTEWYNVKMELLWKIKTEL
EKNGIEIPFPQRVVWFANELRANVEGKEERRQA.

By “MscSfam polynucleotide” is meant a nucleic acid molecule encoding an MscSfam polypeptide. In particular embodiments, the codons of the MscSfam polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscSfam polynucleotide is provided immediately below. In some embodiments, the MscSfam polynucleotide is the nucleic acid molecule as identified by its Genbank Accession number or a functional fragment, ortholog, or homolog thereof. In some embodiments, the MscSfam polynucleotide is substantially identical to the nucleic acid molecule identified by the Genbank Accession number or a variant, ortholog, or homolog having substantial identity thereto. In some embodiments, the MscSfam polynucleotide comprises or consists of base pairs 1393480 to 1394331 (locus tag: AF_1546) of the nucleic acid molecule identified by the Genbank Accession number AE000782.1. In some embodiments, the MscSfam polynucleotide is a functional homolog, ortholog, or fragment of base pairs 1393480 to 1394331 (locus tag: AF_1546) of the nucleic acid molecule identified by the Genbank Accession number AE000782.1. In some embodiments, the MscSfam polynucleotide contains the MscSfam polynucleotide sequence provided immediately below. In some embodiments, the MscSfam polynucleotide consists essentially of the MscSfam polynucleotide sequence provided immediately below.

### MscSfam Polynucleotide Sequence:

(SEQ ID NO: 22)
GTGGGGATTATGATTGACGTGCTGAATTACAAGCTGTATGGAGACGTCAC
GGTTTACGACATTATCGTCGTTATTGTCGTCATGGCCCTTGCCACAATAA
TTGCAAAGCTCATAACAACCAACCTAAGGAGGGCTCTGATTGACAAAATG
AAGAGGGACCAGCTTGAGCTGATGCTCAAGGTTATCTACTTCGGAATAAT
CATCGTTGCCTTCATCGCAGTTTTGCCGGCTCTGGGCCTTGACCTTTCAG
GTTTACTGGTTGCCGGTGGAATTACCGGAATTGTTCTAGGTTTTGCCAGC
CAGAGCGTCGTTGCAAACCTCGTTTCGGGAATCTTCCTGATCTCGGAGAA
ACCGATAAAAATTGGAGATCAGGTTAACATTGACGGTGTGGCGGGCTTCG
TCGAGGATGTTAACATCCTATCAACCATTATAAGAACCTACGATGGTTTG
TACGTCAGAATCCCAAACGAGAAGGTCTTCACGTCCAACATAACCAACTA
CGTCGCCCACATCGCAAGGAGGTTTGAGTACGTTGTGGGCATTAGGTACA
GCGATGATGCTGAGAAAGCCATCGAGATAATAAAAAGAATTATTGAGGAG
CACCCCTTCGCCCTGAAAAATCCCGAGCCCGTGGTTTTCGTTGACAATCT
TGGGGATAGCAGTGTGAACATCGTCGTGAGAATCTGGGCTCCCTCGACGG
AGTGGTACAACGTGAAGATGGAGCTTCTCTGGAAGATAAAGACTGAGCTT
GAGAAGAACGGAATCGAGATACCTTTCCCGCAGCGTGTGGTGTGGTTTGC
CAACGAGTTGAGAGCCAATGTTGAGGGTAAGGAAGAGAGACGGCAGGCT.

In some embodiments, for expression in a mammalian cell, e.g., a human cell, the following codon-optimized MscSfam polynucleotide sequence is used, or a sequence with at least 85% sequence identity thereto is used. In some embodiments, a sequence with at least 90%, at least 95%, or at least 98% sequence identity thereto is used.

### Codon-Optimized MscSfam (Also Termed Gp178 MscSfam Afu1) Polynucleotide Sequence:

(SEQ ID NO: 23)
ATGGGCATCATGATCGACGTGCTGAACTACAAGCTGTATGGCGACGTGAC
CGTGTACGACATCATCGTGGTCATCGTGGTCATGGCCCTGGCCACAATCA
TCGCCAAGCTGATCACCACAAATCTGCGGAGAGCCCTGATCGACAAGATG
AAGCGGGATCAGCTGGAGCTGATGCTGAAAGTGATCTACTTCGGCATCAT
CATCGTGGCCTTTATCGCCGTGCTGCCCGCCCTGGGCCTGGATCTGTCCG
GACTGCTGGTGGCAGGAGGAATCACCGGCATCGTGCTGGGCTTCGCCTCT
CAGAGCGTGGTGGCCAACCTGGTGTCCGGCATCTTTCTGATCTCTGAGAA
GCCCATCAAGATCGGCGACCAGGTGAACATCGATGGCGTGGCCGGCTTCG
TGGAGGACGTGAATATCCTGAGCACCATCATCAGGACATACGATGGCCTG
TATGTGCGCATCCCTAATGAGAAGGTGTTCACCTCCAACATCACAAATTA
CGTGGCCCACATCGCCAGGCGCTTTGAGTACGTGGTGGGCATCAGGTATT
CTGACGATGCCGAGAAGGCCATCGAGATCATCAAGCGCATCATCGAGGAG
CACCCTTTCGCCCTGAAGAACCCCGAGCCTGTGGTGTTTGTGGACAACCT
GGGCGATAGCTCCGTGAATATCGTGGTGAGAATCTGGGCCCCAAGCACCG
AGTGGTATAACGTGAAGATGGAGCTGCTGTGGAAGATCAAGACAGAGCTG
GAGAAGAATGGCATCGAGATCCCATTCCCCCAGCGGGTGGTGTGGTTTGC
CAACGAGCTGAGAGCCAATGTGGAGGGCAAGGAGGAGCGGAGACAGGCC.

By “MscS-like polypeptide” is meant a mechanosensory protein derived from Bacillus halodurans that is capable of conferring ultrasound sensitivity on a cell, e.g., a neuron, and having at least about 85% sequence identity to the MscS-like polypeptide sequence provided below, a fragment thereof, or a human ortholog thereof, and having the biological activity described herein. In embodiments, the mechanosensory protein has at least about 90%, at least about 95%, or at least about 98% sequence identity to the MscS-Like polypeptide sequence provided below, a fragment thereof, or a human ortholog thereof, and has the biological activity described herein. In other embodiments, the polypeptide is the polypeptide identified by its Genbank Accession number or a functional fragment, isoform, ortholog, or homolog thereof. In some embodiments, the MscS-like polypeptide is substantially identical to the protein identified by the Genbank Accession number or a variant, isoform, ortholog, or homolog having substantial identity thereto. In some embodiments, the MscS-like polypeptide is the polypeptide identified by the Genbank Accession number BAB06402.1. In some embodiments, the MscS-like polypeptide is a homolog or functional fragment of the polypeptide identified by the Genbank Accession number BAB06402.1. In some embodiments, the MscS-like polypeptide contains the MscS-like polypeptide sequence provided immediately below. In some embodiments, the MscS-like polypeptide consists essentially of the MscS-like polypeptide sequence provided immediately below.

### MscS-Like Polypeptide Sequence:

(SEQ ID NO: 24)
MNSYYARQWERFVQSFERFNWQDYLIPVAIFIIFLVFRKVFTKYIFKLIL
RLAKKSKTDVLTNLLLSFEKPLRAFWIILGTYLALMALPFPVTAVAFVDH
LYRSLLILLLGWGFFNYTAAHSTIFLTIAKWMDLDENSMVIPFLSKMLRF
IVVALTILIILAEWEFKIGGFIAGLGLGGLAFALAAQDTIGNFFGGVIIV
TEKPFSKGDWIQTPTVEGVVEDITFRSTRVRTFADSVVTVPNSTLASEPI
TNWSQMRKRRITFNLGLEYATTKEQLQSVRTKIEAYLRQHDQVDQEVIMV
HFSEFNSSSLDIFIYFFTNTIVWSEWYVVKEEINLKIIEILEEEGVSVAF
PSRSVYMKRESEGEILPPQKALESKEKDQ.

By “MscS-like polynucleotide” is meant a nucleic acid molecule encoding an MscS-like polypeptide. In particular embodiments, the codons of the MscS-like polynucleotide are optimized for expression in an organism of interest or in the cells of an organism of interest (e.g., optimized for human expression or expression in human cells, bacterial expression or bacterial cell expression, mammalian expression or mammalian cell expression). The sequence of an exemplary MscS-like polynucleotide is provided immediately below. In some embodiments, the MscS-like polynucleotide is the nucleic acid molecule as identified by its Genbank Accession number or a functional fragment, ortholog, or homolog thereof. In some embodiments, the MscS-like polynucleotide is substantially identical to the nucleic acid molecule identified by the Genbank Accession number or a variant or homolog having substantial identity thereto. In some embodiments, the MscS-like polynucleotide comprises or consists of base pairs 2803820 to 2804959 (gene: BH2683) of the polynucleotide identified by the Genbank Accession number BA000004.3. In some embodiments, the MscS-like polynucleotide is a functional homolog, ortholog, or fragment of base pairs 2803820 to 2804959 (gene: BH2683) of the polynucleotide identified by the Genbank Accession number BA000004.3. In some embodiments, the MscS-like polynucleotide contains the MscS-like polynucleotide sequence provided immediately below. In some embodiments, the MscS-like polynucleotide consists essentially of the MscS-like polynucleotide sequence provided immediately below.

### MscS-Like Polynucleotide Sequence:

(SEQ ID NO: 25)
TTGGTCTTTTTCTTTTGATTCTAACGCTTTTTGCGGAGGTAAGATCTCAC
CCTCTGATTCTCGCTTCATATACACGCTGCGGCTCGGGAACGCAACAGAT
ACTCCTTCTTCCTCTAAAATCTCGATGATTTTTAAGTTGATCTCTTCTTT
CACCACATACCATTCAGACCAGACGATGGTGTTCGTAAAGAAATAGATAA
AAATATCGAGGCTAGAGGAGTTAAATTCACTAAAATGAACCATGATCACC
TCTTGATCCACTTGATCGTGCTGACGCAAATAGGCTTCAATTTTCGTTCG
CACCGATTGGAGCTGCTCTTTCGTCGTTGCGTATTCAAGTCCAAGGTTAA
ACGTAATCCGGCGTTTTCGCATCTGGGACCAGTTCGTAATCGGTTCACTT
GCAAGCGTTGAGTTCGGAACGGTTACCACCGAATCCGCAAACGTGCGAAC
TCTCGTGCTACGGAACGTTATATCTTCAACCACACCCTCAACAGTTGGCG
TCTGGATCCAATCCCCTTTCGAGAATGGCTTTTCAGTCACGATAATGACC
CCGCCGAAAAAATTGCCGATCGTATCTTGTGCGGCCAAGGCAAACGCCAA
TCCCCCTAAGCCTAGACCTGCGATAAAGCCACCGATCTTAAACTCCCATT
CCGCAAGGATGATCAGAATGGTGAGGGCCACGACAATAAACCGTAGCATT
TTGGACAAAAACGGGATAACCATACTGTTTTCATCAAGATCCATCCATTT
GGCAATTGTTAAGAAGATCGTCGAATGGGCCGCTGTATAATTAAAAAATC
CCCATCCTAAAAGTAAAATTAATAGCGAACGGTACAAGTGGTCTACAAAG
GCGACAGCTGTCACGGGGAACGGCAAAGCCATAAGAGCTAAGTATGTTCC
GAGAATAATCCAAAACGCCCGCAACGGCTTCTCAAAGGAAAGTAACAAGT
TTGTAAGGACGTCGGTTTTCGACTTTTTCGCTAGCCGTAAAATAAGCTTA
AAAATATACTTCGTAAATACTTTTCGAAATACAAGAAAGATGATAAAAAT
CGCAACGGGTATAAGATAGTCTTGCCAGTTAAACCGTTCAAACGATTGAA
CAAAACGTTCCCATTGTCTCGCGTAATATGAATTCAT.

In some embodiments, for expression in a mammalian cell, e.g., a human cell, the following codon-optimized MscS-like polynucleotide sequence is used, or a sequence with at least 85% sequence identity thereto is used. In some embodiments, a sequence with at least 90%, at least 95%, or at least 98% sequence identity thereto is used.

### Codon-Optimized MscS-Like (Also Termed Gp177 Msclik Bha1) Polynucleotide Sequence:

(SEQ ID NO: 26)
ATGAACAGTTACTATGCAAGGCAGTGGGAGCGGTTTGTCCAGTCATTTGA
ACGGTTTAATTGGCAGGATTATCTGATTCCAGTGGCTATCTTCATCATCT
TTCTGGTGTTCCGGAAGGTGTTTACCAAGTACATCTTCAAGCTGATCCTG
AGACTGGCCAAGAAGTCTAAGACCGATGTGCTGACAAACCTGCTGCTGAG
CTTTGAGAAGCCCCTGAGGGCCTTCTGGATCATCCTGGGCACATATCTGG
CCCTGATGGCACTGCCATTTCCTGTGACCGCAGTGGCCTTCGTGGACCAC
CTGTACCGCTCCCTGCTGATCCTGCTGCTGGGCTGGGGCTTCTTTAACTA
TACAGCCGCCCACTCTACCATCTTTCTGACAATCGCCAAGTGGATGGACC
TGGATGAGAACAGCATGGTGATCCCTTTTCTGTCTAAGATGCTGCGGTTC
ATCGTGGTGGCCCTGACCATCCTGATCATCCTGGCCGAGTGGGAGTTTAA
GATCGGCGGCTTCATCGCAGGCCTGGGCCTGGGAGGCCTGGCCTTTGCCC
TGGCCGCCCAGGATACAATCGGCAATTTCTTTGGCGGCGTGATCATCGTG
ACCGAGAAGCCTTTCTCCAAGGGCGACTGGATTCAGACCCCAACAGTGGA
GGGCGTGGTGGAGGACATCACCTTTCGGAGCACAAGGGTGCGCACCTTCG
CCGACTCCGTGGTGACAGTGCCAAACTCTACCCTGGCCAGCGAGCCCATC
ACAAATTGGAGCCAGATGAGAAAGCGGAGAATCACCTTCAACCTGGGCCT
GGAGTACGCCACCACAAAGGAGCAGCTGCAGAGCGTGAGGACCAAGATCG
AGGCCTATCTGCGCCAGCACGACCAGGTGGATCAGGAGGTGATCATGGTG
CACTTTAGCGAGTTCAACAGCTCCTCTCTGGACATCTTCATCTACTTCTT
TACCAACACAATCGTGTGGTCCGAGTGGTACGTGGTGAAGGAGGAGATCA
ATCTGAAGATCATCGAGATCCTGGAGGAGGAGGGCGTGAGCGTGGCATTC
CCCAGCCGGTCCGTGTACATGAAGAGAGAATCCGAAGGGGAAATCCTGCC
ACCTCAGAAAGCACTGGAAAGCAAAGAGAAAGATCAG.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

By “behavioral alteration” is meant a change in how a subject acts, reacts, or responds to stimulation. In some embodiments, the behavioral alteration may involve an alteration in eating habits, manner of interaction with other subjects (e.g., aggression, generosity, avoidance, increase in quality of interaction, etc.), tendency toward self-harm, tendency to cause damage (e.g., damage to property or objects), tendency toward obsessive or compulsive actions, sexual activity, manner of socializing with others, behavior toward drugs (e.g., drug addiction), tendency to abuse or take regulated substances (e.g., alcohol or drugs), pathological or risky behaviors (e.g., cheating, stealing, gambling, sexual promiscuity, etc.), or tendency to harm others physically or emotionally.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide is implicit in each described sequence with respect to the expression product, but not with respect to actual probe sequences.

By “altered” is meant an increase (or enhancement), or a decrease (or reduction). An increase is any positive change, e.g., by at least about 5%, 10%, or 20%; or by about 25%, 50%, 75%, or even by 100%, 200%, 300% or more. A decrease is a negative change, e.g., a decrease by about 5%, 10%, or 20%; or by about 25%, 50%, 75%; or even an increase by 100%, 200%, 300% or more.

The terms “comprises”, “comprising”, and are intended to have the broad meaning ascribed to them in U.S. Patent Law and can mean “includes”, “including” and the like.

“Contacting” is used in accordance with its plain ordinary meaning and refers to the process of allowing at least two distinct species (e.g. chemical compounds including biomolecules, reagents, or cells) to become sufficiently proximal to react, interact, effect, affect or physically touch. It should be appreciated, however, that the resulting reaction product can be produced directly from a reaction between the added reagents or from an intermediate of one or more of the added reagents, which can be produced in the reaction mixture or under the contacting conditions. Contacting may include allowing two species to react, interact, or physically touch, wherein the two species may be a recombinant viral particle as described herein and a cell. In some embodiments, the two species are an ultrasound contrast agent that is exposed to ultrasound and a cell. In some embodiments, the two species are ultrasound and a cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The term “exogenous” (synonymous with “heterologous”) refers to a molecule, reagent, or substance (e.g., a compound, nucleic acid (polynucleotide) or protein (polypeptide or peptide) that originates or derives from a source outside of a given cell or organism. For example, an “exogenous promoter” as referred to herein is a promoter that does not originate from the source (e.g., a given cell or organism) in which it is expressed. By way of example, an “exogenous” or “heterologous” polypeptide or polynucleotide as referred to herein does not originate from the source (e.g., a given cell, tissue, organ, or organism) in which it is expressed, but is obtained or derived from a different source and is introduced or delivered into a given cell, tissue, organ, or organism by genetic or recombinant techniques and then is expressed in that given cell, tissue, organ, or organism. By way of example, an exogenous promoter may be derived from a given organism, such as a bacterium, plant, or fungus (yeast), and used in another organism or cell type, such as a mammalian cell. Conversely, the term “endogenous” (e.g., “endogenous promoter,” “endogenous protein or polypeptide,” or “endogenous polynucleotide”) refers to a molecule or substance that is native to, or originates within, a given cell, tissue, organ, or organism.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” or “homologous.” This definition also refers to, or may be applied to, the compliment of a test sequence. The definition also includes sequences that have deletions and/or additions, as well as those that have substitutions. As described below, the preferred algorithms can account for gaps and the like. In some embodiments, sequence dentity exists over a region that is at least about 25 amino acids or nucleotides in length, or over a region that is 50-100 amino acids or nucleotides in length.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, polypeptide, or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule as described is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide as described that has been separated from components that naturally accompany it. Typically, the polypeptide is isolated when it is at least 50%, at least 55%, or at least 60%, by weight, free from the proteins and naturally-occurring organic molecules with which it is naturally associated. In embodiments, a preparation is at least 75%, or at least 90%, or at least 99%, by weight, a mechanosensory polypeptide as described herein. An isolated polypeptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “mammal” is meant any warm-blooded animal including, but not limited to, non-human primate (monkey, ape, baboon and the like), human, cow, horse, pig, sheep, goat, mouse, rat, dog, cat, and the like. In an embodiment, the mammal is a human.

The terms “mechanosensitive”, “mechanically activated”, “mechanoreceptor”, “mechanotransduction”, “stretch-gated”, “acoustically sensitive”, and other similar terms of art as used herein are considered interchangeable and are used to refer to a cell, tissue, or polypeptide, or other material object that is sensitive to activation or inactivation by acoustical energy, such as ultrasound.

By “modulating” is meant effecting or altering the activity or function of a cell, tissue, organ, organism, or subject, for example, by subjecting the a cell, tissue, organ, organism, or subject, to ultrasound stimulation. In an embodiment, the activity or function of a neuronal cell is modulated by applying or delivering ultrasound or ultrasound waves to the neuronal cell. In embodiments, modulating neuronal cells in a subject, e.g., in the brain or CNS of the subject, affects the subject's behavior or response to an agent, stimulus, situation, effect, or activity. In some embodiments, modulating an activity or function may cause an increase or decrease in an the subject's response or responsiveness. In some embodiments, modulating an activity or function may cause an increase (enhancement) or decrease (inhibition) of cell activity or function, or in a subject's response or responsiveness.

“Nucleic acid” refers to deoxyribonucleotides or ribonucleotides and polymers thereof in either single- or double-stranded form, or complements thereof. The term “polynucleotide” refers to a linear sequence of nucleotides. The terms “polynucleotide” and “nucleic acid molecule” are used interchangeably herein. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and 2-O-methyl ribonucleotides.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term, “obtaining” as in “obtaining an agent” includes synthesizing, deriving, isolating, purchasing, or otherwise acquiring the agent, e.g., a protein, polynucleotide, or sample.

By “positioned for expression” is meant that a polynucleotide (e.g., a DNA molecule) is positioned adjacent to a DNA sequence, which directs transcription, and, for proteins, translation of the sequence (i.e., facilitates the production of, for example, a recombinant polypeptide as described or provided herein, or an RNA molecule).

The term “plasmid” or “vector” refers to a nucleic acid molecule that encodes for genes and/or regulatory elements necessary for the expression of genes. Expression of a gene from a plasmid or vector can occur in cis or in trans. If a gene is expressed in cis, the gene and the regulatory elements are encoded by the same plasmid and vector. Expression in trans refers to the instance where the gene and the regulatory elements are encoded by separate plasmids or vectors.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 5%, 10%, 25%, 50%, 75%, or 100%.

By “reference” or “control” is meant a standard condition. For example, an untreated cell, tissue, or organ that is used as a reference.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. For specific polypeptides described herein (e.g., MscS, MscL, MscK, MscL G22S, MscS-like, MscMJ, MscMJLR, MscS-Like 3, MscSfam), the named polypeptide includes any of the polypeptide's naturally occurring forms, or variants, isoforms, or homologs that maintain the polypeptide's mechanosensory activity (e.g., within at least 50%, 80%, 90%, 95%, 96%, 97%, 98%, 99% or 100% activity compared to the native polypeptide). In some embodiments, variants or homologs have at least 90%, 95%, 96%, 97%, 98%, 99% or 100% amino acid sequence identity across the whole sequence or a portion of the polypeptide sequence (e.g. a 50, 100, 150 or 200 continuous amino acid portion) compared to a naturally occurring form. In other embodiments, the polypeptide is the polypeptide as identified by its Genbank Accession number.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “sonogenetics” or “sonogenics” refers to a non-invasive approach, method, or technique to manipulate, control, or modulate the activity or function of a cell or cell type, such as a neuron (e.g., a motor neuron), that expresses a heterologous or exogenous mechanosensitive (also called mechanotransductive) channel, which is responsive to ultrasound, e.g., low-intensity ultrasound. Cell activity, such as neuronal cell activity, can be controlled or modulated by expressing a heterologous or exogenous mechanosensitive channel in a target cell, e.g., a neuron or type of neuronal cell such as a motor neuron, and subjecting the cell to ultrasound (low-intensity ultrasound), which is thereby responsive to ultrasound or ultrasound pulses. In an embodiment, the cell types are located within the mammalian brain. Target cells that express such heterologous or exogenous mechanosensitive channel is specific cells renders those cells sensitive to mechanical deformations generated by noninvasive ultrasound waves. In an embodiment, the cells are neurons in regions of the brain or in the spinal cord (central nervous system, CNS). In an embodiment the cells are in peripheral nervous system (PNS). In an embodiment, the region of the brain is the hypothalamus. In an embodiment, ultrasound is delivered or applied to the hypothalamus using an external transducer. In an embodiment, the transducer is non-invasively positioned on the head of an awake mammalian subject. In an embodiment, the transducer is a PZT-based transducer. In an embodiment, the cells are neurons in the spinal cord.

The term “subject” as used herein refers to a vertebrate, such as a mammal (e.g., dog, cat, rodent, horse, bovine, rabbit, goat, non-human primate, or human). In an embodiment, a subject is a human individual or patient.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, or at least 80% or 85%, or at least 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide as described and provided herein.

As used herein, the terms “treat,” treating,” “treatment,” and the like refer to reducing, abating, decreasing, ameliorating, or eliminating a disease, disorder, or condition and/or symptoms associated therewith. It will be appreciated that, although not precluded, treating a disease, disorder, or condition does not require that the disease, disorder, condition or symptoms associated therewith be completely eliminated.

By “transformed cell” is meant a cell into which (or into an ancestor of which) has been introduced, by means of recombinant DNA techniques, a polynucleotide molecule encoding (as used herein) a polypeptide as described and provided herein.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

An “effective amount” is an amount sufficient to accomplish a stated purpose (e.g. achieve the effect for which it is administered, treat a disease, reduce enzyme activity, reduce one or more symptoms of a disease, disorder, or condition, or reduce viral replication in a cell). An example of an “effective amount” is an amount sufficient to contribute to the treatment, prevention, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, disorder, pathology or condition, or reducing the likelihood of the onset (or reoccurrence) of an injury, disease, disorder, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein (e.g. Tat, Rev) relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient,” “subject” or “subject in need thereof” refers to a living organism or individual suffering from, afflicted with, having, at risk for, or susceptible or prone to, a disease, pathology, disorder, or condition that can be treated by using the products, compositions and methods provided herein. The term does not necessarily indicate that the subject has been diagnosed with a particular disease or disorder, but typically refers to an individual under medical supervision. Non-limiting examples include humans, other mammals, bovines, rats, mice, dogs, monkeys, goat, sheep, cows, deer, as well as other non-mammalian animals. In some embodiments, a patient or subject is human.

The terms “ultrasonic wave”, “acoustical energy”, “acoustic wave”, or “ultrasound” are used interchangeably herein to refer to the disturbance in a material corresponding to the mechanical transfer or mechanical transduction of energy through the material. In various embodiments, the disturbance is a vibration of the materials' components. In some embodiments, the material is a volume of liquid, a cell, a cell membrane, a tissue, or an organ.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The recitation of a listing of chemical groups in any definition of a variable herein includes definitions of that variable as any single group or combination of listed groups. The recitation of an embodiment for a variable or for an aspect herein includes that embodiment as any single embodiment or in combination with any other embodiments or portions thereof as described herein.

Any compositions or methods provided herein can be combined with one or more of any of the other compositions and methods provided herein.

### BRIEF DESCRIPTION OF THE DRAWINGS

FIGS. 1A and 1B provide a schematic and images showing expression of bacterial mechanosensory proteins in mouse brain. FIG. 1A is a schematic showing the injection site for viral vectors containing polynucleotides encoding the bacterial mechanosensory proteins. The areas of the brain shown in FIG. 1A include the mammillary nucleus (MM); periaqueductal gray area (PAG); substantia nigra-pars compacta (SNc); substantia nigra-pars reticulata (SNr); red nucleus (RN); medial lemniscus (ml); cerebral peduncle (cp); and ventral tegmental area (VTA). FIG. 1B is a collection of images of the rodent brain showing that the indicated microbial proteins were expressed in the ventral tegmental area (VTA). Expression was observed to be restricted to dopaminergic neurons.

FIGS. 2A to 2D provide a schematic, recordings and a graph relating to sonogenetics in the spinal cord. FIG. 2A is a schematic showing the position of the ultrasound transducer for targeting bacterial mechanosensory protein-expressing motor neurons in the spinal cord, and the recording electrode in the downstream gastrocnemius muscle. FIGS. 2B and 2C are recordings of electrical impulses showing that ultrasound elicited evoked muscle potentials when motor neurons that expressed MscS were targeted (FIG. 2B), but not when control motor neurons that expressed green fluorescent protein (GFP) were targeted (FIG. 2C). FIG. 2D is a graph showing that ultrasound-evoked muscle potentials (dark grey circles, at 10 ms pulse from a 385 KHz transducer) matched those elicited by electrical stimuli (white-filled circles, 0.05 ms electrical pulse) for the parameters of duration and latency. EMG refers to “electromyography” and USSC refers to “ultrasound sensitive channel.” In FIGS. 2B and 2C, the upper line represents the ultrasound signal intensity over time in seconds, and the lower line represents the intensity of electrical signal in the muscle over time in seconds. In FIGS. 2B and 2C, the vertical axis represents signal intensity and the horizontal axis represents time in seconds. The horizontal axis is 200 ms and the y-axis is 200 μV.

FIGS. 3A and 3B provide a schematic, images, and bar graphs relating to sonogenetics in the brain of an animal. The left panel of FIG. 3A is a schematic showing injection sites for delivering an adeno-associated virus vector harboring a polynucleotide encoding the MscS polypeptide (AAV-MScS) or an adeno-associated virus vector encoding green fluorescent protein (AAV-GFP) into the arcuate nucleus. The right panels of FIG. 3A provides images showing expression of the MscS polypeptide and GFP in AGRP-positive (AGRP-^{+ve}; agouti-related protein-expression positive) neurons in the arcuate nucleus of mouse brain. FIG. 3B provides bar graphs showing that animals expressing the MscS protein in AGRP^{+ve} (agouti-related protein-expression positive) neurons consumed an increased amount of food (based on a behavioral feeding assay) upon ultrasound stimulation compared to food consumption by control animals expressing GFP.

### DETAILED DESCRIPTION OF THE EMBODIMENTS

Featured and provided herein are compositions featuring bacterial mechanosensory polypeptides and polynucleotides, methods for expressing such polypeptides and polynucleotides in a cell type of interest, and methods for inducing the activation of the bacterial mechanosensory polypeptide in neuronal cells (e.g., neurons) and other cell types using ultrasound.

The aspects and embodiments described herein are based, at least in part, on the discovery that specific proteins, e.g., mechanosensory proteins, confer sensitivity to ultrasound when transduced or transfected into target cells and expressed as heterologous proteins on the target cell surface. The Examples provided herein demonstrate that such mechanosensory proteins have a sensitivity to ultrasound. Not wishing to be bound by theory, ultrasound can generate a mechanical deflection in the focal zone that leads to activation of the expressed mechanosensory protein. In embodiments described and exemplified herein, the mechanosensory proteins can be used non-invasively to control neuronal cells, namely, motor neurons and ARGP(+ve) hypothalamic neurons in the rodent spinal cord and brain. The products, compositions and methods described herein allow for precision targeting of neuronal stimulation at the neuron level. The non-invasive nature of ultrasound permit long-term use of the described mechanosensory proteins for treatment of diseases and disorders, including neurological and neurodegenerative diseases and disorders, and/or the symptoms thereof, such as, without limitation, essential tremor, Parkinson's disease, ataxia, and pain. In an embodiment, the disease, disorder, or condition is an eating disorder.

In various embodiments, the described heterologous mechanosensory proteins can be used to render neurons, cardiac muscle, urinary bladder tissues, T-cells, or beta-cells responsive to ultrasound. In some embodiments, the mechanosensory proteins can be used to render sensitive to ultrasound any cell type that is sensitive to a rapid change in cation (e.g., calcium, potassium, sodium) concentration. The mechanosensory proteins can be used to alter cellular functions in vivo, in vitro, or ex vivo. In addition, the mechanosensory proteins can be used to alter cell function of cells expressing these proteins in cell culture.

Accordingly, provided and described herein are polynucleotides encoding a bacterial mechanosensory polypeptide, expression vectors comprising such polynucleotides, cells expressing a heterologous bacterial mechanosensory polypeptide, cells expressing a heterologous recombinant bacterial mechanosensory polypeptide, and methods for stimulating such cells with ultrasound.

### Ultrasound

Ultrasound is well suited for stimulating neuron populations as it focuses easily through intact thin bone and deep tissue (K. Hynynen and F. A. Jolesz, Ultrasound Med Biol 24 (2), 275 (1998)) to volumes of just a few cubic millimeters (G. T. Clement and K. Hynynen, Phys Med Biol 47 (8), 1219 (2002)). The non-invasive nature of ultrasound stimulation is particularly significant for manipulating vertebrate neurons including those in humans, as it eliminates the need for invasive techniques, such as surgery, to insert light fibers (required for some current optogenetic methods). Also, the small focal volume of the ultrasound wave compares well with light that is scattered by multiple layers of brain tissue (S. I. Al-Juboori et al., PLoS ONE 8 (7), e67626 (2013)). Moreover, ultrasound has been previously used to manipulate deep nerve structures in human hands and reduce chronic pain (W. D. O'Brien, Jr., Prog Biophys Mol Biol 93 (1-3), 212 (2007); L. R. Gavrilov et al., Prog Brain Res 43, 279 (1976)). As described herein, novel, non-invasive compositions for the heterologous expression of bacterial mechanosensory polypeptides in cells are provided, and methods to stimulate or modulate the activity or function of cells expressing heterologous bacterial mechanosensory polypeptides using low-intensity ultrasound stimulation are provided. In an embodiment, the cells that express the heterologous bacterial mechanosensory polypeptides are neuronal cells, and the methods involve neuromodulation by the non-invasive use of ultrasound stimulation, in particular, low-intensity ultrasound.

### Cells and Cellular Compositions Comprising Recombinant Bacterial Mechanosensory Polypeptides

Provided are cells comprising a recombinant nucleic acid molecule encoding a bacterial mechanosensory polypeptide. Such mechanosensory polypeptides of bacterial origin are heterologously or exogenously expressed in a cell type of interest. In an embodiment, the cell type of interest expresses a heterologous bacterial mechanosensory polypeptide as described herein and is sensitive to a rapid change in cation (e.g., calcium, potassium, sodium) concentration associated with mechanosensory modulation or stimulation of the cell by ultrasound. In an embodiment, the cell type is a cardiac muscle cell comprising a bacterial mechanosensory polynucleotide under the control of a promoter suitable for expression in a cardiac cell (e.g., NCX1 promoter). In an embodiment, the cell type is a muscle cell comprising a bacterial mechanosensory polynucleotide under the control of a promoter suitable for expression in a muscle cell, e.g., myoD promoter. In another embodiment, the cell type is an insulin secreting cell (e.g., a beta (B) islet cell) comprising a bacterial mechanosensory polynucleotide under the control of a promoter suitable for expression in an insulin-secreting cell, e.g., Pdx1 promoter. In another embodiment, an adipocyte comprising a bacterial mechanosensory polynucleotide under the control of a promoter suitable for expression in an adipocyte (e.g., iaP2) is provided. In another embodiment, the cell type is a neuron comprising a bacterial mechanosensory polynucleotide under the control of a promoter suitable for expression in a neuronal cell. In an embodiment and by way of nonlimiting example, the neuronal cell may be a neuron in the central nervous or peripheral nervous system, a neuron in the brain or spinal cord, a motor neuron, a sensory neuron, an interneuron, an Agouti-Related Protein-expression positive (AGRP-^{+ve}) neuron. By way of nonlimiting examples, a nestin or Tuj 1 promoter is generally suitable for expression of the bacterial mechanosensory polynucleotide in a neuron; an H2b promoter is suitable for expression of the bacterial mechanosensory polynucleotide in a motor neuron; an Islet 1 promoter is suitable for expression of the bacterial mechanosensory polynucleotide in an interneuron; and an OMP promoter, T1R, T2R promoter, rhodopsin promoter, or Trp channel promoter is suitable for expression of the bacterial mechanosensory polynucleotide in a sensory neuron. Such cells may be cells in vitro, ex vivo, or in vivo. In particular embodiments, the cells express a bacterial mechanosensory polypeptide that is sensitive to ultrasound. In particular embodiments, the mechanosensory polypeptide is selected from a MscS, MscL, MscK, MscL G22S, MscS-like, MscMJ, MscMJLR, MscS-Like 3, or MscSfam polypeptide, or a functional portion, isoform, ortholog, or homolog thereof. In another embodiment, the mechanosensory polypeptide, e.g., a MscS, MscL, MscK, MscL G22S, MscS-like, MscMJ, MscMJLR, MscS-Like 3, or MscSfam polypeptide, or a functional portion, isoform, ortholog, or homolog thereof, or the polynucleotide encoding the mechanosensory polypeptide and the like, is codon-optimized for expression in a mammalian cell. In another embodiment, the mechanosensory polypeptide, e.g., a MscS, MscL, MscK, MscL G22S, MscS-like, MscMJ, MscMJLR, MscS-Like 3, or MscSfam polypeptide, or a functional portion, isoform, ortholog, or homolog thereof, or the polynucleotide encoding the mechanosensory polypeptide and the like, is codon-optimized for expression in a mammalian cell or a human cell.

### Expression of Recombinant Bacterial Mechanosensory Polypeptides

In one approach, a cell of interest (e.g., a neuron, such as a motor neuron, sensory neuron, neuron of the central or peripheral nervous system, or neuronal cell lines) is genetically or recombinantly engineered to express a heterologous bacterial mechanosensory polynucleotide whose expression renders the cell responsive to ultrasound stimulation. Ultrasound stimulation of such cells induces cation influx. The molecular techniques involved in genetically or recombinantly engineering cells to express heterologous polypeptides is well known to and routinely practiced by those having skill in the art.

The bacterial mechanosensory polypeptide may be constitutively expressed or its expression may be regulated by an inducible promoter or other control mechanism where conditions necessitate highly controlled regulation or timing of the expression of a bacterial mechanosensory polypeptide. For example, heterologous DNA encoding a bacterial mechanosensory polypeptide gene to be expressed is inserted in one or more pre-selected DNA sequences. This can be accomplished by homologous recombination or by viral integration into the host cell genome. The desired gene sequence can also be incorporated into a cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into cells and is a standard method of DNA transfer to those of skill in the art. DEAE-dextran transfection, which is also known to those of skill in the art, may be preferred over calcium phosphate transfection where transient transfection is desired, as it is often more efficient. Since the cells as described herein are isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells can also be genetically modified using electroporation.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[1-(2, 3-dioleyloxy)propyl]-N—N—N-trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA into the de-differentiated cells or reprogrammed cells described herein.

Naked plasmid DNA can be injected directly into a tissue comprising cells of interest. Microprojectile gene transfer can also be used to transfer genes into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells as described herein and their progeny. Viral vectors are used, as are the physical methods previously described, to deliver one or more polynucleotide sequences encoding the bacterial mechanosensory polypeptides, for example, into the cells. Viral vectors and methods for using them to deliver DNA to cells are well known to those of skill in the art. Examples of viral vectors that can be used to genetically alter the cells as described herein include, but are not limited to, adenoviral vector, adeno-associated viral vectors (AAV), retroviral vectors (including lentiviral vectors), alpha viral vectors (e.g., Sindbis vectors), and herpes virus vectors.

### Targeted Cell Types

Bacterial mechanosensory polypeptides can be expressed in virtually any eukaryotic or prokaryotic cell of interest. In one embodiment, the cell (or target cell) is a bacterial cell or other pathogenic cell type. In another embodiment, the cell (or target cell) is a mammalian cell, such as an adipocyte, muscle cell, cardiac muscle cell, insulin secreting cell (e.g., beta (B) islet cell), pancreatic cell, a glial cell, or neuron (e.g., a motor neuron, a sensory neuron, a neuron of the central nervous system (e.g., a neuron in the brain or in a region of the brain, e.g., the hypothalamus), a neuron of the peripheral nervous system, an interneuronal cell, and neuronal cell lines or populations). Other cell types include, without limitation, immune cells, such as cells of the hematopoietic lineages, T cells, B cells, monocytes, macrophages, stem cells, pluripotent stem cells, induced pluripotent stem cells, primary cells, established cells or cell lines.

### Methods of Stimulating a Neuronal Cell

The methods provided herein are, inter alia, useful for the stimulation (activation) of cells that express the bacterial mechanosensory polypeptides. In particular, ultrasound stimulation of such cells induces cation influx, thereby altering cell activity. Expression of a bacterial mechanosensory polypeptide in a pathogen cell (bacterial cell) and subsequent ultrasound stimulation induces cation influx and bacterial cell killing. Ultrasound stimulation of a muscle cell expressing a heterologous bacterial mechanosensory polypeptide results in muscle contraction. This can be used to enhance muscle contraction or functionality in subjects in need thereof, including subjects suffering from muscle weakness, paralysis, or muscle wasting. Altering the intensity of the ultrasound modulates the extent of muscle activity.

The terms “neuron,” “neural cell,” or “neuronal cell” are used interchangeably herein and refer to a cell of the brain or nervous system, such as the central nervous system (CNS) or the peripheral nervous system (PNS). Non-limiting examples of neural or neuronal cells include neurons, interneurons, astrocytes, oligodendrocytes and microglia cells. Where a neural cell is stimulated, a function or activity (e.g., excitability) of the neural cell is effected by modulating, for example, the expression or activity of a given gene or protein (e.g., a bacterial mechanosensory polypeptide) within the neural cell. The change in expression or activity may be 10%, 20%, 30%, 40%, 50%, 60%, 70%, 80%, 90% or more in comparison to a control (e.g., unstimulated cell). In certain instances, expression or activity is 1.5-fold, 2-fold, 3-fold, 4-fold, 5-fold, 10-fold or higher than the expression or activity in the absence of stimulation. In certain instances, expression or activity is 1.5-fold, 2-fold, 3-fold, 4-fold, 5-fold, 10-fold or lower than the expression or activity in the absence of stimulation. The neural cell may be stimulated by applying an ultrasonic wave to the neural cell.

The term “applying” as provided herein is used in accordance with its plain ordinary meaning and includes the terms contacting, introducing and exposing. An “ultrasonic wave” as provided herein is an oscillating sound pressure wave having a frequency greater than the upper limit of the human hearing range. Ultrasound (ultrasonic wave) is thus not separated from “normal” (audible) sound by differences in physical properties, only by the fact that humans cannot hear it. Although this limit varies from person to person, it is approximately kilohertz (20,000 hertz) in healthy, young adults. Ultrasound (ultrasonic wave) devices operate with frequencies from 20 kHz up to several gigahertz. The methods provided herein use the energy of an ultrasonic wave to stimulate a neural cell expressing an exogenous mechanosensory protein. A mechanotransduction protein as provided herein refers to a cellular protein capable of converting a mechanical stimulus (e.g., sound, pressure, movement) into chemical activity. Cellular responses to mechanosensation or mechanotransduction are variable and give rise to a variety of changes and sensations. In some embodiments, the bacterial mechanosensory protein is a mechanically gated ion channel, which makes it possible for sound, pressure, or movement to cause a change in the excitability of a cell (e.g., a sensory neuron). The stimulation of a bacterial mechanosensory protein may cause mechanically sensitive ion channels to open and produce a transduction current that changes the membrane potential of a cell.

In one aspect, a method of stimulating a cell is provided. The method includes transfecting or transducing a cell with a recombinant vector or viral vector containing a nucleic acid sequence encoding an exogenous (heterologous) bacterial mechanosensory polypeptide, wherein the transfected or transduced cell expresses the mechanosensory polypeptide, in particular, in the cell membrane. An ultrasonic wave is applied to the transfected or transduced cell, thereby stimulating the cell. In some embodiments, the bacterial mechanosensory polypeptide is MscS, MscL, MscK, MscL G22S, MscS-like, MscMJ, MscMJLR, MscS-Like 3, MscSfam, or a homolog or ortholog thereof. In some embodiments, the mechanotransduction polypeptide is a bacterial mechanosensory polypeptide or a functional portion, homolog, or ortholog thereof. In some embodiments, the ultrasonic wave has a frequency of about 0.8 MHz to about 4 MHz. In some embodiments, the ultrasonic wave has a frequency of about 1 MHz to about 3 MHz. In some embodiments, the ultrasonic wave has a focal zone of about 1 cubic millimeter to about 1 cubic centimeter. In some embodiments, the ultrasonic wave has a frequency of about 385 KHz. In some embodiments, the ultrasonic wave has a frequency of about 10 MHz. In some embodiments, the ultrasonic wave has a frequency of about or of at least about 0.001 MHz, 0.01 MHz, 0.1 MHz, 0.2 MHz, 0.3 MHz, 0.4 MHz, 0.5 MHz, 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, 15 MHz, 20 MHz, 30 MHz, 40 MHz, or 50 MHz. In some embodiments, the ultrasonic wave has a frequency of less than about 0.001 MHz, 0.01 MHz, 0.1 MHz, 0.2 MHz, 0.3 MHz, 0.4 MHz, 0.5 MHz, 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, 15 MHz, 20 MHz, 30 MHz, 40 MHz, or 50 MHz. In some embodiments, the ultrasonic wave has a frequency of from about 0.2 MHz to about 20 MHz, from about 0.15 MHz to about 0.6 MHz, from about 0.3 MHz to about 0.4 MHz, from about 9 MHz to about 11 MHz, or of from about 5 MHz to about 20 MHz. In some embodiments, the ultrasonic wave has a frequency of from about 0.1 MHz to about 20 MHz. In some embodiments, the ultrasonic wave has a frequency of from 0.1 MHz to 20 MHz. In some embodiments, the ultrasonic wave has an intensity of less than 500 mW/cm².

In some embodiments, the ultrasonic wave has an intensity of from about or at least about 0.01 W/cm², 0.5 W/cm², 1 W/cm², 5 W/cm², 10 W/cm², 25 W/cm², 50 W/cm², 100 W/cm², 150 W/cm², 200 W/cm², 250 W/cm², 300 W/cm², or 400 W/cm². In some embodiments, the ultrasonic wave has an intensity of less than about 0.01 W/cm², 0.5 W/cm², 1 W/cm², 5 W/cm², 10 W/cm², 25 W/cm², 50 W/cm², 100 W/cm², 150 W/cm², 200 W/cm², 250 W/cm², 300 W/cm², or 400 W/cm². In some embodiments, the ultrasonic wave produces a peak negative pressure of from between 0.05 and 3 MPa within a targeted region.

In some embodiments, ultrasonic waves are administered to a cell or tissue in pulses or bursts. In some embodiments, the pulse repetition frequency for the pulses or bursts is from about 0.1 Hz to about 200 Hz, or from about 0.5 Hz to about 2 Hz. In some embodiments, the pulse repetition frequency is about 1 Hz. In various embodiments, the ultrasonic waves are administered with a duty cycle from about 0.005% to about 100%, from about 0.01% to about 50%, from about 0.1% to about 10%, or from about 0.5% to about 2%. In some embodiments, the duty cycle is about 1%. By “duty cycle” is meant the fraction of the time duration of a single on and off cycle of ultrasonic wave administration over which an ultrasonic wave is actively administered.

In some embodiments, the method further includes contacting a transfected or transduced cell, e.g., a neural cell, with an ultrasound contrast agent prior to applying ultrasound. In various embodiments, the ultrasound contrast agent is a microbubble. In certain embodiments, the microbubble has a diameter of from about 0.1 μm, 0.2 μm, 0.3 μm, 0.4 μm, 0.5 μm, 0.75 μm, 1 μm, 1.5 μm, 2 μm, 3 μm, 4 μm, or 5 μm to about 6 μm, 7 μm, 8 μm, 9 μm, 10 μm, 50 μm, or 100 μm. In certain embodiments, the neural cell forms part of an organism. In some embodiments, the organism is a mammal (e.g., non-human primate, human, murine, bovine, ovine, rodent, camelid, feline, canine mammal).

### Generation of Acoustical Energy (Ultrasound)

Various devices may be used to generate an ultrasound wave, such as acoustic and ultrasonic emitters, transducers or piezoelectric transducers, composite transducers, micromachined ultrasound transducers (MUTs) including capacitive micromachined ultrasonic transducers (cMUTs), Micro-Electro-Mechanical Systems (MEMS), silicon on insulator MEMS (SOI MEMS). A device for generating ultrasound waves may be provided as single or multiple transducers or in array configurations. The ultrasound waves may be of any shape, and may be focused or unfocused. Focal spot size depends on probe active aperture diameter (A), wavelength (lambda) and focal length (F). The center deflection of a clamped circular plate under a uniform pressure can be found from the following equation for a circular membrane

$$P = \frac{{Eh}^{4}}{{R}^{3}} [ \frac{16 y}{3 ( 1 - {v}^{2} ) h} + \frac{( 7 - v ) {y}^{3}}{3 ( 1 - {v}^{2} ) {h}^{3}} + \frac{4 {R}^{2} σ y}{( 1 - v ) {Eh}^{3}} ]$$

where P is the uniform pressure applied on the membrane, R is the membrane radius, y is the center deflection, σ is the intrinsic stress of the membrane material, E is the Young's modulus of the membrane material, and v is Poisson's ratio of the membrane material. This equation can be used to estimate the pressure of the ultrasound waves under a prescribed membrane deflection. Such emitters may be made atop a substrate. Multiple substrates may be combined to form a single applicator. Multiple applicators may be combined to form a single probe.

In some embodiments, an ultrasonic wave is generated by an opto-acoustic system or transducer, such as that described, for example, in U.S. Pat. No. 6,022,309 and U.S. Pat. Appl. Pub. No. 20050021013, the entire disclosures of which are incorporated herein by reference in their entirety for all purposes. In some embodiments, ultrasonic waves are generated by optical energy delivered by pulsed laser light, optionally guided through an optical fiber. In some embodiments, the optical fiber is disposed within a catheter. In some embodiments, optical energy is deposited in a water-based optical energy-absorbing fluid, e.g. saline, thrombolytic agent, blood or thrombus, and generates an acoustic impulse in the fluid through thermoelastic and/or thermodynamic mechanisms. By pulsing the laser at a repetition rate (e.g., from 10 Hz to 100 kHz) ultrasonic waves can be established locally in the medium. In some embodiments, a high repetition rate laser system is used to produce the optical energy. In various embodiments, the laser light has a pulse frequency within the range of from about 10 Hz to about 100 kHz, or in the range of from about 20 kHz to about 1,000 kHz, or in the range of from about 0.1 MHz to about 50 MHz, or from about 0.1 MHz to about 20 MHz, or from about 0.2 MHz to about 10 MHz, or from about 1 MHz to about 20 MHz, or a wavelength within the range of about 200 nm to about 5000 nm and an energy density within the range of about 0.01 J/cm² to about 4 J/cm². In one embodiment, the pulse frequency is within the range of about 5 kHz to about 25 kHz. In various embodiments, an optical fiber used to deliver optical energy to a fluid has a core diameter of 200 microns or of 100 microns or less.

Not wishing to be bound by theory, an absorbing fluid responds thermoelasticly to the deposition of the optical energy such that a region of high pressure is created in the fluid in a volume of a composition (e.g., a fluid, blood, a tissue, a cell, a composition comprising cells, etc.) heated by the optical energy. The boundary of the high pressure zone decays into a pattern of acoustic waves and a compression wave propagates away from the energy deposition region (diverging wave front) and a rarefaction wave propagates towards the center of the energy deposition region (converging wave front). When the rarefaction wave converges on the center of the initial deposition region, it creates a region of tensile stress that promotes the formation of a cloud of cavitation bubbles which coalesce to form a larger bubble. Eventually, the cavitation bubble collapses resulting in an expanding acoustic wave. Collapse and subsequent rebound of the cavitation bubble will generate acoustic waves in the surrounding fluid, which will carry off a portion of the energy of the cavity. The collapse and rebound processes take place on a time scale governed principally by the fluid density and the maximum size of the initial cavity. The first collapse and rebound will be followed by subsequent collapse and rebound events of diminishing intensity until the energy of the cavity is dissipated in the fluid. In some embodiments, subsequent laser pulses are delivered to repeat or continue this cycle and generate ultrasonic waves at a frequency or frequencies determined by the laser pulse frequency.

The pulsed laser energy source used as described herein is not limiting and can be based on, as non-limiting examples, a gaseous, liquid or solid state medium. Rare earth-doped solid state lasers, ruby lasers, alexandrite lasers, Nd:YAG (neodymium-doped yttrium aluminum garnet; Nd:Y3A15012) lasers and Ho:YLF (neodymium-doped yttrium lithium fluoride) lasers. Any of these solid state lasers may incorporate non-linear frequency-doubling or frequency-tripling crystals to produce harmonics of the fundamental lasing wavelength. A solid state laser producing a coherent beam of ultraviolet radiation may be employed directly with the products, compositions, and methods as described herein or used in conjunction with a dye laser to produce an output beam which is tunable over a wide portion of the ultraviolet and visible spectrum.

In one aspect, an ultrasonic wave may be generated in a fluid by: (i) depositing laser energy in a volume of the fluid comparable to a dimension (e.g., diameter or a maximum dimension of an area over which laser energy is absorbed by the fluid) of an optical fiber used to deliver the laser energy and in a time scale of duration less than the acoustic transit time across the dimension (as controlled by, for example, choice of laser wavelength and/or absorbing fluid); (ii) controlling the laser energy such that the maximum size of a generated cavitation bubble is approximately the same as the fiber dimension; and (iii) pulsing the laser at a repetition rate such that multiple cycles of this process generate an acoustic radiation field in the fluid. Resonant operation may be achieved by synchronizing the laser pulse repetition rate with cavity lifetime. In some embodiments, operation leads to a fluid-based transducer that cycles at 1-100 kHz with a reciprocating displacement of 100-200 μm. In various embodiments, the acoustic waves are propagated into tissue or fluid surrounding the small volume of fluid.

In another aspect, an ultrasonic wave may be generated in a fluid by: (i) depositing laser energy in a small volume of fluid (as controlled by, for example, choice of laser wavelength and absorbing fluid) within a blood vessel or circumvented by a tissue; (ii) controlling the laser energy such that the maximum size of a vapor bubble generated is approximately the same as or less than the diameter of a blood vessel within which the vapor bubble is generated or the diameter defined by the circumventing tissue within which the vapor bubble is generated; and (iii) pulsing the laser energy at a repetition rate such that multiple cycles of the bubble generation and collapse process generates an acoustic waves in the fluid. In various embodiments, the acoustic waves are propagated into tissue or fluid surrounding the small volume of fluid.

### Methods of Treatment and Cellular Manipulation

In one aspect, non-invasive therapeutic methods are provided, which use sound waves (ultrasound) to activate mechanosensitive cellular transmembrane proteins (mechanoreceptors) that in turn excite, increase, or inhibit cellular function, locally and/or downstream of the initial source of ultrasound application. In another aspect, a method of altering the function of a sensory unit that innervates a targeted tissue portion of an animal is provided. In another aspect, a method of treating or ameliorating a neurological or neurodegenerative disease or disorder in a subject in need thereof is provided. In another aspect, a method for manipulating the activity or function of a cell or tissue is provided, in particular, a mammalian cell or tissue, including a human cell or tissue. The methods include expressing in a cell or tissue of a subject a therapeutically effective amount of an exogenous mechanosensory polypeptide (e.g., MscS, MscL, MscK, MscL G22S, MscS-like, MscMJ, MscMJLR, MscS-Like 3, MscSfam) and applying ultrasound (an ultrasonic wave) to the cell or tissue, thereby resulting in a change in mechanosensory polypeptide conductance, e.g., cation influx, in the cell or tissue. In an embodiment, the methods include administering or delivering to a cell or tissue of a subject a therapeutically effective amount of a recombinant nucleic acid, a vector, or a viral vector encoding an exogenous mechanosensory polypeptide (e.g., MscS, MscL, MscK, MscL G22S, MscS-like, MscMJ, MscMJLR, MscS-Like 3, MscSfam) and applying ultrasound (an ultrasonic wave) to the cell or tissue, resulting in a change in mechanosensory polypeptide conductance, e.g., cation influx, in the cell or tissue. In an embodiment, the methods involving application of ultrasound to a cell are used to manipulate a tissue or cell ex vivo, in vivo, in situ, or ex situ, or to modulate the activity or function of the cell. In an embodiment, the methods involve treating or ameliorating a cardiac disease by modulating, e.g., altering, controlling, enhancing, or increasing, cardiac muscle activity. In an embodiment, the methods involve treating or ameliorating a neurological disease or disorder by modulating, e.g., altering, controlling, enhancing, or increasing, neural or neuronal cell activity in the subject. In some embodiments, the neurological disease or disorder is Parkinson's disease, depression, obsessive-compulsive disorder, chronic pain, epilepsy or cervical spinal cord injury. In some embodiments, the method can include the manipulation of neurons (e.g., deep brain stimulation or other applications), cardiac muscle (e.g., a pacemaker), urinary bladder, T-cells (e.g., in treatment of cancer or an immune disease), beta-cells (e.g., insulin production), or a combination thereof. In embodiments, the neurological disease is retinal degeneration or atrial fibrillation. In some embodiments, the neurological disease is a neurodegenerative disease, for example, Alzheimer's disease, Parkinson's disease, motor neuron diseases, Huntington's disease, spinocerebellar ataxia (SCA), or spinal muscular atrophy (SMA). In some embodiments, the methods involve treating or ameliorating an eating disorder. In particular embodiments, the exogenous mechanosensory polypeptide is a bacterial mechanosensory polypeptide, namely, MscS, MscL, MscK, MscL G22S, MscS-like, MscMJ, MscMJLR, MscS-Like 3, MscSfam, or an ortholog or homolog or functional portion thereof.

In some embodiments, the method further includes administering to the subject, cell, or tissue an ultrasound contrast agent prior to the application of ultrasound to the subject, cell, or tissue. In some embodiments, the ultrasound contrast agent is a microbubble. In some embodiments, the microbubble has a diameter of from about 0.1 μm, 0.2 μm, 0.3 μm, 0.4 μm, 0.5 μm, 0.75 μm, 1 μm, 1.5 μm, 2 μm, 3 μm, 4 μm, or 5 μm to about 6 μm, 7 μm, 8 μm, 9 μm, 10 μm, 50 μm, or 100 μm, and is injected or otherwise introduced into the body (e.g., the brain), tissue, cell, or culture medium containing the tissue or cell where it enhances ultrasound stimulation.

In various embodiments, the method involves delivering or applying acoustical energy (ultrasound) to a targeted tissue or cell. In general, the delivery or application of the acoustical energy to the targeted tissue or cell is non-invasive. In some embodiments the target tissue or cell forms part of a sensory unit. In various embodiments, the tissue or cell has been configured to express an acoustically sensitive transmembrane protein, e.g., an exogenous or heterologous acoustically sensitive transmembrane protein, such that when the tissue or cell is exposed to acoustical energy, a membrane potential of cell or cells comprising the tissue is modulated at least in part due to exposure of the acoustically sensitive protein to the acoustical energy. The tissue or cell may have been genetically or recombinantly modified to express the acoustically sensitive transmembrane protein. The acoustically sensitive transmembrane protein may be selected from MscS, MscL, MscK, MscL G22S, MscS-like, MscMJ, MscMJLR, MscS-Like 3, MscSfam, or an ortholog or homolog thereof

The acoustical source may be selected from, as non-limiting examples, a piezoelectric transducer, e.g., a PZT-based transducer, a composite transducer, a micromachined ultrasound transducer, a capacitive micromachined ultrasonic transducer, and a micro-electro-mechanical system. The acoustical source may comprise a silicon-on-insulator type micro-electro-mechanical system. In some embodiments, the acoustical energy is delivered transcutaneously from an acoustical source. In some embodiments, the acoustical energy is delivered transcranially. In some embodiments, the tissue or cell is in the brain, e.g., the hypothalamus, of a subject. In some embodiments, the tissue or cell constitutes part of the central nervous system. In some embodiments, the tissue or cell constitutes part of the peripheral nervous system. In some embodiments, the acoustical source is disposed on or within a tissue or organ of the subject. In some embodiments, the acoustical source is disposed on or within the brain of the subject. In a particular embodiment, a nerve cell expressing an acoustically sensitive, heterologous bacterial transmembrane protein as described herein, e.g., a motor neuron, in one area, e.g., the spinal cord, is stimulated with acoustical energy (ultrasound), resulting in activation of nerve cells, e.g., motor neurons, in another area, e.g., in downstream muscle tissue. In some embodiments, the method involves using a plurality of acoustic emitters to activate cells or tissue(s) of a subject. In embodiments, the cells are neurons, e.g., motor neurons, in the spinal cord and/or in downstream muscles.

In an aspect, a method for sonogenetics-based neuromodulation in a patient is provided. In some embodiments, the method involves determination of a desired nervous system functional modulation which can be facilitated by sonogenetic excitation and/or inhibition. The method can further involve a selection of a neuroanatomic resource within the patient to provide such outcome. The method can involve causing cells of the neuroanatomic resource to render them sensitive to acoustical energy. The method can further involve delivering acoustical energy to the targeted neuroanatomy to cause controlled, specific excitation and/or inhibition of such neuroanatomy by virtue of the presence of the mechanoresponsive protein in cells thereof. In some embodiments, bacterial mechanosensory proteins modulate membrane potential of a neuron, or other type of cell, by transporting ions, e.g., potassium, sodium, or calcium ions, through the membrane of the cell.

### Compositions and Pharmaceutical Compositions

Compositions comprising cells expressing a heterologous or exogenous bacterial mechanosensory polypeptide as described herein are provided. In an embodiment, the composition is a pharmaceutical composition. Typically, the carrier or excipient for such a composition is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, and the like.

A composition or pharmaceutical composition is administered at a dosage or effective amount that ameliorates, decreases, diminishes, abates, alleviates, or eliminations the effects of a disease, disorder, or condition, and/or the symptoms thereof. The composition may be provided in a dosage form that is suitable for parenteral (e.g., subcutaneous, intravenous, intramuscular, intrathecal, or intraperitoneal) administration route. The pharmaceutical compositions may be formulated and prepared according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). A pharmaceutical composition may be administered parenterally by injection, infusion or implantation (subcutaneous, intravenous, intramuscular, intraperitoneal, intrathecal, and the like). Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation.

The practice of the aspects and embodiments herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides as described herein, and, as such, may be considered in making and practicing the described products, compositions and methods. Particularly useful techniques for particular embodiments will be discussed in the sections that follow.

The following examples are put forth so as to provide those of ordinary skill in the art with a complete disclosure and description of how to make and use the assay, screening, and therapeutic methods as described and provided herein, and are not intended to be limiting.

### EXAMPLES

### Example 1: Expression of Mechanosensory Polypeptides in the Mouse Brain

MscS, MscL, MscK, MscL G22S, MscS-like, MscMJ, MscMJLR, MscS-Like 3, and MscSfam polypeptides were identified as candidate bacterial mechanosensory proteins for expression in neurons in the mouse brain. The mechanosensitive channel of small conductance (MscS), the mechanosensitive channel of large conductance (MscL), and MscK (potassium-dependent mechanosensitive channel) are derived from Escherichia coli, MscS-like is from Bacillus halodurans, MscMJ and MscMJLR are from Methanocaldococcus jannaschii, MscS-Like 3 is from Arabidopsis thaliana (A. thaliana), and MscSfam is from Archaeoglobus fulgidus. In particular, MscS is a transmembrane protein that functions as a nonspecific mechanosensitive channel in the bacterial cell and opens in response to pressure inside the bacterial cell (turgor pressure), relieving the pressure before the cell ruptures. MscS is a hepatamer with three transmembrane helices and a large cytoplasmic soluble domain. The MscS polypeptide constitutes 286 amino acids and is one of four mechanosensitive channels in E. coli that play a role in osmoregulation. MscS is responsive to membrane pressures of 5-8 mN/m and does not require any other cellular structures for gating function. When in its open conformation, the pore is 13 Å in diameter and conducts at 1 nS. Other members of the MscS channel family can be as large as 1,120 amino acids.

Nucleotide sequences encoding the polypeptides were codon optimized for expression in human cells. Polynucleotides, including codon-optimized polynucleotide sequences, encoding the MscS, MscL, MscK, MscMJ, and MscMJLR polypeptides were synthesized and cloned into adeno-associated virus (AAV) vectors for CRE-dependent (cyclization recombinase-dependent) expression in neuronal cells in vitro or in vivo. In particular, the polynucleotide sequences encoding the codon-optimized polypeptides for expression in cells, including human cells, such as neurons in the brain and CNS, are as set forth supra and in SEQ ID NOS: 3, 6, 9, 11, 14, 17, 20, 23 and 26.

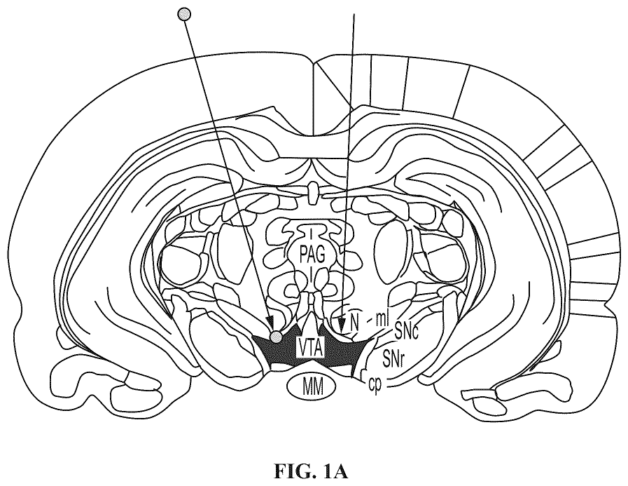

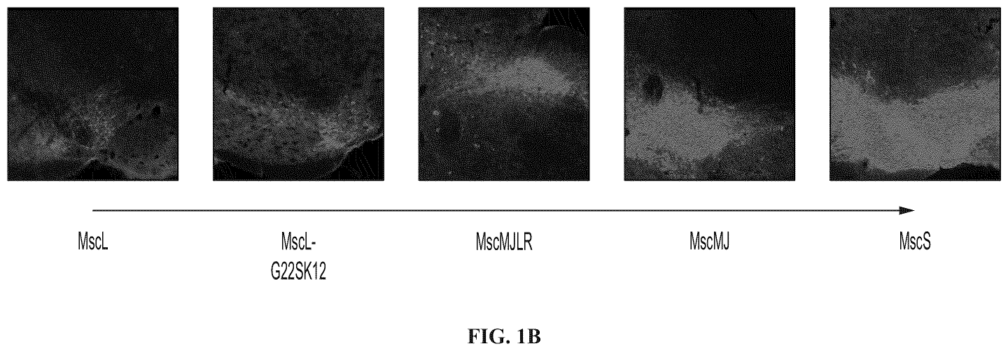

The virus vectors were then injected into the ventral tegmental area of a mouse that expressed CRE (cyclization recombinase) in dopaminergic neurons. As shown in FIGS. 1A and 1B, the bacterial polypeptides were expressed in mouse brain cells following injection.

### Example 2: Manipulation of Neurons Using a Mechanosensory Polypeptide

Studies were performed to express heterologous bacterial mechanosensory proteins in the central nervous system (CNS), e.g., in neurons, e.g., neurons within the brain, or in the spinal cord, of animals (mice), subject the neurons in the animals to ultrasound, and assess the resulting activity in the animals via electromyography, a technique that measures muscle response or electrical activity in response to a nerve's stimulation of the muscle.

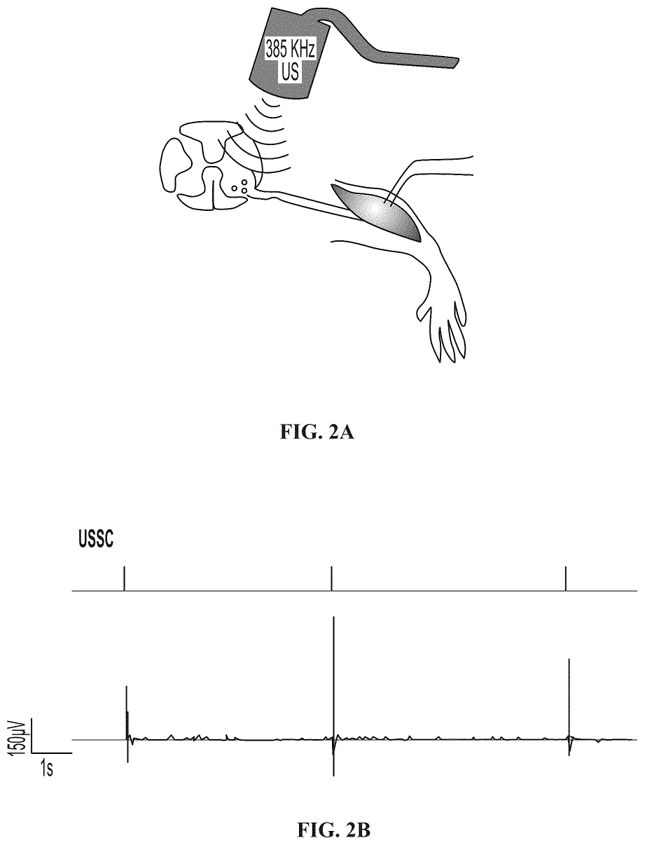

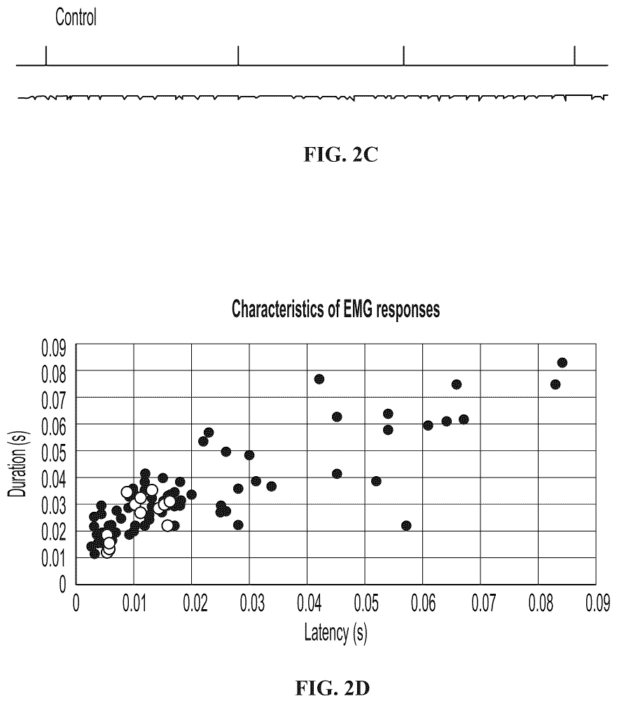

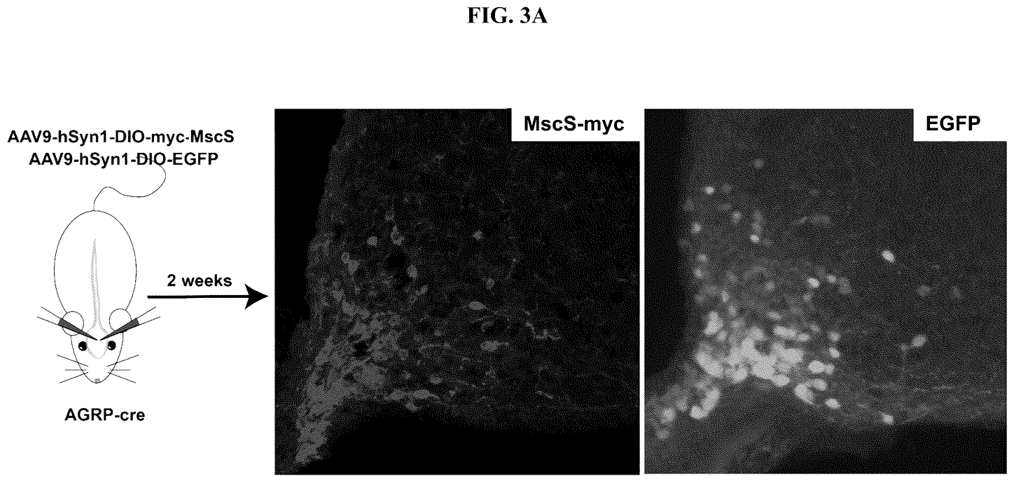

Bacterial small conductance mechanosensitive channel (MscS) channel proteins were expressed in motor neurons in the spinal cord of mice by stereotaxic injection of AAVs (adeno-associated virus vectors) containing polynucleotides encoding MscS channel proteins into mice whose motor neurons specifically express CRE (cyclization recombinase) (FIG. 2A). Electromyography was used to monitor animals expressing mechanosensory proteins in motor neurons (FIGS. 2A to 2D). In anesthetized animals, activating motor neurons using a 385 KHz ultrasound transducer, but not a 10 MHz transducer, resulted in electrical signals in downstream muscles (FIGS. 2B and 2C). Moreover, the time course and latency of the ultrasound-evoked muscle potentials matched those elicited by electrical impulses, suggesting that sonogenetics can be used to non-invasively control motor neurons in vivo (FIGS. 2B to 2D). Without intending to be bound by theory, ultrasound is likely causing a complex change in the cell membrane, e.g., deflection, and a change in membrane capacitance, which is detected by the sonogenetic channels.

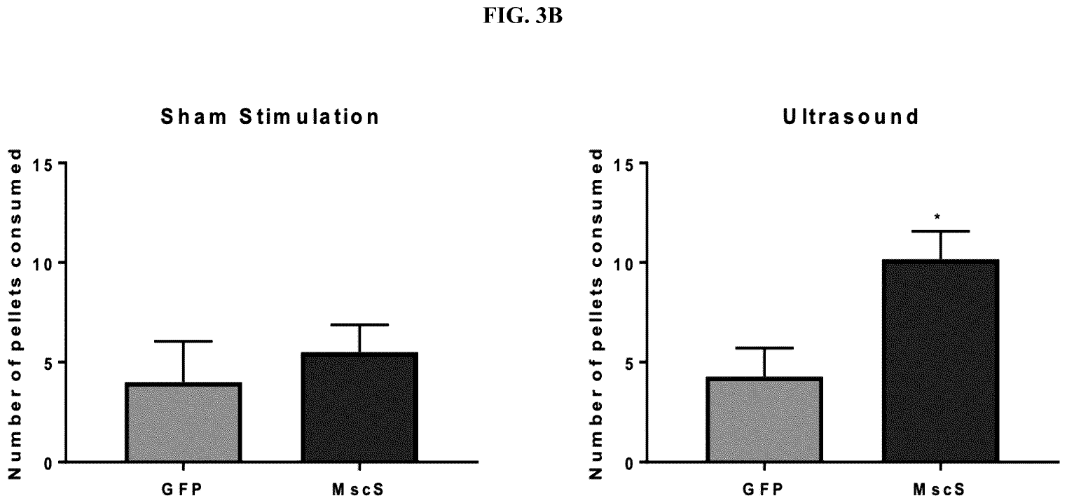

In addition, a small PZT-based transducer (a piezoelectric transducer of lead zirconate titanate (PZT); XMS-310B Panametrics, Olympus Corporation) was designed to be positioned on the head of an awake, behaving animal, e.g., a mouse (8-12 weeks old), to deliver ultrasound to the hypothalamus. AAV-MscS (adeno-associated virus vectors encoding MscS) were injected into the arcuate nucleus of an AGRP-CRE (agouti-related protein—cyclization recombinase) mouse, and expression of the channel in AGRP+ve (agouti-related protein positive) neurons was evaluated and confirmed (FIG. 3A). A feeding assay was used to test whether mice expressing MscS in AGRP+ve (agouti-related protein positive) neurons ate increased amounts of food in response to ultrasound at 10 MHz (FIG. 3B). The feeding assay was carried out as described in Aponte, et al., “AGRP neurons are sufficient to orchestrate feeding behavior rapidly and without training”, Nature Neuroscience, 14:351-355 (2011), which is incorporated herein in its entirety by reference for all purposes.

The above results demonstrate that MScS and other microbial proteins encoded by human codon-optimized polynucleotides are heterologously expressed in cells in the mouse brain and have the ability to manipulate and control neurons (motor neurons), such as neuronal behavior and function, in the spinal cord and hypothalamus in a non-invasive manner upon application of ultrasound to the neuronal cells.

### Example 3: Expression of Codon-Optimized Mechanosensory Polypeptides in Human Cells

In addition to the expression of the described mechanosensitive proteins in neurons within the mouse brain, the codon-optimized polynucleotides encoding mechanosensitive proteins (polypeptides) were expressed in human cells. Such non-naturally occurring, codon-optimized polynucleotide sequences encoding the polypeptides were synthesized and cloned into viral vectors for CRE-dependent human cell expression. In particular and by way of example, codon-optimized polynucleotide sequences encoding the mechanosensitive polypeptides for expression in human cells are as set forth supra and in SEQ ID NOS: 3, 6, 9, 11, 14, 17, 20, 23 and 26.

### Example 4: Materials and Methods

The materials and methods described below were used in the above-described examples.

### Viruses and Injections

An adeno-associated virus vector harboring a polynucleotide encoding a mechanosensitive bacterial protein as described herein, namely, AAV9-hsyn1-DIO-myc-mechanosensitive protein-encoding polynucleotide, was injected at 5e12 or maximum possible titer along with 1e12 AAV9-hsyn1-DIO-GFP (Addgene #100043-AAV9) diluted in Hank's Balance Salt Solution when necessary to achieve the desired amount of virus. Male and female ˜8-10 week old DAT-cre mice (Jax stock 006660) were injected unilaterally at coordinates AP-3.2 ML-0.65 DV-4.3 (ventral tegmental area) with 750 nL virus. Injections were performed through small holes drilled in the skull at the appropriate coordinates, and virus was delivered through a glass needle attached to a Nanoject iii (Dummond Scientific Company) at 3 nL/s. After five minutes, the needle was removed, incision was sutured, and the mouse was allowed to recover for 3 days, followed by 3 weeks incubation for virus expression. Expression was confirmed via sacrifice and immunohistochemistry for the myc tag on the encoded protein.

### Immunohistochemistry and Imaging

After a 3 week incubation, mice were perfused and fixed with 0.9% saline and 4% paraformaldehyde (PFA) through a peristaltic pump. Brains were immediately removed and soaked in 4% paraformaldehyde (PFA) overnight, then moved to 30% sucrose solution for 48 hours. Each brain was sectioned at 35 μm/section into tissue collection solution (glycerol, ethylene glycol, phosphate buffered saline (PBS)). Brain sections containing ventral tegmental area (VTA) were stained by immunohistochemistry for myc-tag using tyramide signal amplification (TSA). Briefly, tissue was quenched for 30 minutes (min) in hydrogen peroxide, and then blocked with phosphate buffered saline (PBS)/10% TRITONX/5% Horse Serum for 1 hour. Sections were mutated overnight in rabbit anti-myc antibody (1:500 dilution; Cell Signaling 2272S), followed by a 3 hour room temperature (RT) incubation in biotinylated donkey anti-rabbit antibody (1:500 dilution; Jackson Immunoresearch 711-065-152). Sections were then washed in phosphate buffered saline (PBS), incubated for 30 min in ABC (Vector Labs PK-4000), washed in phosphate buffered saline (PBS), incubated for 30 min in tyramide, washed in PBS, and mutated (2.5 hours RT) in streptavidin conjugated Alexa-647 (Thermo Fischer S21374). Finally, sections were stained with 1:1000 DAPI (4′,6-diamidino-2-phenylindole) and washed in phosphate buffered saline (PBS) before being mounted onto glass slides to which a cover slip was applied with Prolong Gold Antifade Mounting Medium (Thermo Fischer Scientific).

Images of myc labeling corresponding to protein expression were collected on a Zeiss Airyscan 880 confocal microscope. Consistent imaging settings were maintained throughout to compare relative expression levels of each candidate protein.

### Feeding Assay

AGRP-cre (agouti-related protein—Cre recombinase) mice were acclimated to feeding behavior boxes prior to viral injections. Briefly, mice were placed in a plexiglass cage (12″×12″×18″) with smooth walls containing only a wall-mounted pellet feeder filled with 0.2 g pellets (Precision Pellets). Mice underwent a 5-day training protocol in which they were fasted overnight, but food was restored throughout the day immediately after training. Day 1: mice were pair acclimated with 20 pellets placed on the floor (30 min). Day 2: mice were singly acclimated with 10 pellets placed on the floor (30 min). Day 3-4: Mice were singly acclimated with food only in the pellet feeder (30 min). On Day 5, mice were placed in the chamber and number of pellets consumed over 30 minutes was recorded. Only mice that consumed greater than 10 pellets during training were used for behavioral studies.

Acclimated AGRP-cre mice (males and females about 8 weeks of age) were injected with AAV9-hSyn-DIO-GFP or AAV9-hSyn-DIO-MscS targeting the arcuate nucleus of the hypothalamus. Two weeks later, a metal ferrule was attached to the skull via dental cement. After a 1-week recovery period, mice underwent a sham training session in which the ultrasound transducer was attached to their head (under brief isoflurane anesthesia) via the ferrule and mice were allowed to recover and explore the feeding boxes for 45 minutes.

For testing, mice were briefly anesthetized while the transducer was attached. Mice were then placed in the behavior boxes and allowed to recover. Testing was conducted in blocks as follows: 0-5 min: No ultrasound, 5-35 min: intermittent ultrasound stimulation or sham treatment: 35-50 min: no ultrasound. Ultrasound was delivered as 1-second-long bursts comprising 20 ms on, 30 ms off ultrasound, with 4 seconds without stimulation between bursts. Number of pellets consumed were recorded in real time and via video monitoring in 5-minute bins. At the end of the session, remaining pellets were weighed to ensure that accurate tallies were taken. Sham and ultrasound treatment sessions were randomized. At least 2 days elapsed between testing sessions.

### Electromyography (EMG):

Adeno-associated virus (AAVs) expressing MScS or green fluorescent protein (GFP) controls were injected into ChAT:Cre expressing animals to drive expression of the transgene (heterologous polynucleotide) in motor neurons. Electromyography (EMG) experiments were conducted between 2-4 weeks after viral injection. Electromyography (EMG) data were collected under ketamine (100 mg/kg) and xylazine (10 mg/kg) anaesthesia from the right and left biceps brachii and right and left biceps femoris through fine wire electrodes (A-M Systems 790700) connected to a PowerLab and BioAmp (AD Instruments). Data were collected at 40 k/sec, bandpass filtered from 300 Hz to 1 kHz. Correct electrode placement was confirmed by positive electromyography (EMG) signal in response to pinch. The skin over the skull was opened, and the 6.91 MHz lithium niobate ultrasound transducer was coupled to the skull using ultrasound gel (Parker Aquasonic 100). Ultrasound stimuli (1 ms, ms, 100 ms durations) were administered at no less than 10 second intervals at intensities ranging from 0.35-1.05 MPa, to the lumbar spinal cord. Visual movement of the right fore or hindlimb in response to stimulation was noted and electromyography (EMG) responses were analysed for latency and duration. Only responses occurring after cessation of the stimulus were considered in the analyses. The experimenter was blinded as to the group during both collection and analysis of the data.

### OTHER EMBODIMENTS

From the foregoing description, it will be apparent that variations and modifications may be made to the aspects and embodiments as described herein for adapting to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication were specifically and individually indicated to be incorporated by reference.

What is claimed is:

1. A method of inducing cation influx in a cell, the method comprising:
expressing in the cell a polypeptide selected from MscS, MscL, MscK, MscL G22S, MscMJLR, MscMJ, MscS-Like 3, MscSfam, or MscS-like; wherein the polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and
applying ultrasound to the cell, thereby inducing cation influx in the cell.

2. The method of claim 1, wherein the cell is sensitized to mechanical deformation or stretch caused by ultrasound.

3. A method of rendering a cell responsive to mechanical deformation or stretch caused by ultrasound or sensitizing a cell to mechanical deformation or stretch caused by ultrasound and activating and/or modifying activity or function of the cell, the method comprising:
(a) transducing a cell to express a heterologous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively;
(b) applying ultrasound to the cell; and
(c) inducing cation influx in the bacterial mechanosensory polypeptide expressing cell and an alteration in cell activity and/or function following the application of ultrasound, thereby rendering the cell responsive to mechanical deformation or stretch caused by ultrasound.

4. The method of claim 1, wherein the polynucleotide sequence encoding the bacterial mechanosensory polypeptide is codon-optimized for expression in a mammalian or human cell and is non-naturally occurring.

5. The method of claim 1, wherein the heterologous, bacterial mechanosensory polypeptide is expressed in the cell following transduction of the cell by a plasmid or viral vector which contains the polynucleotide sequence encoding polypeptide.

6. The method of claim 5, wherein the cell is transduced by a viral vector selected from a lentivirus vector or an adeno-associated virus (AAV) vector.

7. The method of claim 1, wherein the cell is one or more of a muscle cell, a cardiac muscle cell, an insulin secreting cell, a glial cell, or a neuronal cell.

8. The method of claim 7, wherein the neuronal cell is selected from a motor neuron, a sensory neuron, an interneuron, or an Agouti-Related Protein-expression positive (AGRP-^{+ve}) neuron.

9. The method of claim 1, wherein the ultrasound has a frequency of about 0.2 MHz to about 20 MHz and/or has a focal zone of about 1 cubic millimeter to about 1 cubic centimeter.

10. A method of treating a disease or disorder in a subject in need thereof, the method comprising:
(i) expressing in a cell of a mammalian subject a heterologous nucleic acid molecule encoding a mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and
(ii) applying ultrasound to the cell, thereby treating the disease or disorder in the subject.

11. A method of treating a disease or disorder in a mammalian subject in need thereof or modulating neuronal activity or function in a subject in need thereof, the method comprising:
(i) transducing into a cell of the subject a polynucleotide molecule encoding an exogenous, bacterial mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively;
(ii) applying ultrasound to the cell; and
(iii) inducing cation influx in the bacterial mechanosensory polypeptide-expressing cell and an alteration in cell activity and/or function following the application of ultrasound, thereby treating the disease or disorder in the subject.

12. The method of claim 10, wherein the cell is one or more of a muscle cell, a cardiac muscle cell, an insulin secreting cell, a glial cell, or a neuronal cell.

13. The method of claim 12, wherein the cell is a neuronal cell.

14. The method of claim 13, wherein the neuronal cell is selected from a motor neuron, a sensory neuron, an interneuron, or an Agouti-Related Protein-expression positive (AGRP-^{+ve}) neuron.

15. The method of claim 10, wherein the ultrasound is applied to the cell in the hypothalamus of the subject.

16. The method of claim 10, wherein the disease or disorder is a neurological disease or disorder, or a neural circuit disease selected from Parkinson's disease, depression, muscle weakness, muscle atrophy, muscle degeneration obsessive-compulsive disorder, eating disorders, chronic pain, epilepsy, spinal injury, anxiety, Alzheimer's, post-traumatic stress disorder (PTSD), or cervical spinal cord injury or muscle weakness, muscle atrophy, muscle degeneration, spinal injury, or cervical spinal cord injury.

17. A method of distal modulation of neuronal activity in a subject in need thereof, the method comprising:
(i) expressing in a neuronal cell of a mammalian subject a heterologous nucleic acid molecule encoding a mechanosensory polypeptide selected from MscS, MscL, MscK, MscL G22S, MscMJLR, MscMJ, MscS-Like 3, MscSfam, or MscS-Like; wherein the mechanosensory polypeptide is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively; and
(ii) applying ultrasound to the neuronal cell at a first site in the subject's nervous system and modulating the activity of neuronal cells at a second site in the subject's nervous system; wherein the second site is distal to the first site.

18. The method of claim 17, wherein the neuronal cells are selected from brain neuron cells, hippocampal neuron cells, or motor neurons.

19. The method of claim 17, wherein the first site is the spinal cord and the second site is muscle tissue downstream of the first site.

20. A plasmid or viral vector comprising a polynucleotide encoding a polypeptide selected from MscS, MscL, MscK, MscL G22S, MscMJLR, MscMJ, MscS-Like 3, MscSfam, or MscS-Like; wherein the polynucleotide is codon-optimized for expression in a mammalian cell and is encoded by a polynucleotide sequence having at least 85% sequence identity to a polynucleotide sequence selected from SEQ ID NO: 3, SEQ ID NO: 6, SEQ ID NO: 9, SEQ ID NO: 11, SEQ ID NO: 14, SEQ ID NO: 17, SEQ ID NO: 20, SEQ ID NO: 23 or SEQ ID NO: 26, respectively.

21. A cell comprising the plasmid or viral vector of claim 20.
